SEQUENCE LISTING (32 sequences)

SEQ ID NO: 1 - DNA, 7163 bp, Micrococcus luteus
gcggagtcctcgtccgcctcggcgtcgtcgctgtccgcggccccggccgactacgaggccggcacgtgcttcaccgccccgctcggcgcgcgtgacctgtcctccttcgagaccaccgactgcgagggcgcccacaccgcggagtacctgtgggccgtgccggccgtggccgagggtgaggaggccgaccccgccgccgcccagacctgcaccgcccaggcccagcgcctgagcgaggagaaggaggaccagctgaacggggccgtcctgacctcctccgagctgggcaactacggcaccgacgagaagcactgcgtcgtgtacggggtctccggtgagtgggagggtcagatcgtggacccggagatcaccctggagacggcgtccgccgacgcctgatcccgccggcggccccgtgcgtcgtgagatcgcgccgcccgggaccgccgcggatggacgcgggaccggcgcggcccgtagtgtcttctgcgtccagaagttagacggtcgaacaggtgcggcggtcggtgccgcgtcgtgtccgccaccgaggaggcgccatgggtgaagcgaggacgggcggcgaggccgcgctctccggggtgaccgccgagctggacgccgcgctccgacacgccgcggcccaggcgcccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgactgcggccggcgagctcgaggacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgccgcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctgagatggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggccgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtccctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtccgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtcgcggcccgcttccaggacccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtggaggtcgcggaccgtcccgcccccgccggccgcctcggacgcctggccgcccgcctgcccaggccgggagcagcccgcggggacgagggccgacgtcgccgcccgggccgggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggggaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggccagggctggcgggtggaccgcgccggggacgcccaggtggaggccgtggcggaccgggccctcgatcagctggcccgctgggccgggatccccgacctggccgagcgcatcgtggtgcggcgcacctacgggcccggtgacttcgccgcggacgtgcacgcctggcggggttcgctgctgggccccgggcacacgctggcgcagtcggccatgttccgcccctcggtgcgggacgcggacgtggccggcctgatgtacgcgggctcctcggtgcgcccgggaatcggggtgcccatgtgcctgatctccgccgaagtggtccgggacgaactgcgccacgacgcgcgcagggcccggcccgcgggccccggggggagcggcacatgatccgcaccctcttctgggtgtcccggccggtcagctgggtgaacacggcctacccgttcgccgccgccgcgatcctgaccggggggctgcccgcgtggctggtggtcctgggcgtcgtgttcttcctggtgccctacaacctggccatgtacggcatcaatgacgtgttcgacttcgcctcggacctgcgcaacccccgcaaggggggtgtggagggctccgtgctgggcgaccccgcggtgcgccgccgggtgctggcgtggtcggtgctgctgcccgtgccgttcgtggccgtgctcgcgggctggtccgccgtgcggggcgagtgggccgccgtgctggtgctcgcggtgagcctgttcgcggtggtggcgtactcctgggcggggctgcggttcaaggagcggcccttcctggacgccgccacctccgccacccacttcgtctcccccgcggtctacggcctcgcgctggccggggcgacccccacgcccgccctggcggcgctgctgggggcgttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccgctcgcggcgctgctggccgtgccctacgtggtgaacaccctgcgcttccgccgcatcacggacgccacctcgggcgcggcccaccgcggctggcagctgttccttccgctgaactacgtgaccggcttcctcgtgaccctgctgctgatcgggtgggcgctgacccggggggcggcggcatgatctacctgctggccctgctgggtgtcatcggctgcatgctgctggtggaccggcgcttcgagctgttcctgtggcatcgcccgctcccggcgctgctggtgctggccgccggggtggcctacttcttcgcctgggacctgtgggggatcgccgaaggcgtgttcctgcaccggcagtcgccctacatgaccggggtgatgctcgccccccagctgcccctggaggaggggttcttcctgctcttcctcagccagatcacgatggtgctgttcaccggggcgctgcgcctgctgcgcggccggcgaggtgacgcccgtgccgcgacggcggccgatccgaccgaccgggggagccggtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtcgtgccgatcggcgcggccgtcggccgggacgccgcccccacccggacgatcgctgacatgctgccgttgatccccgcagacctgctgcgcgcgctcggcctgatcctcgtcccggtcgcggcggtgcacgccggatggccgtccgcggcggcgatgctgctcgtgttcggctcccagtggctcacccgctggctcgccccgggcggcgccctggactgggccgcgcaggcggtcctgctgctggccgggtggctgagcgtcatcggcctctacccgcgggtgccgtggctggacctgctcgtgcacgccgccgcctccgccgtggtcgcctgtctgacggcactggtggtgggggcgtggctccggcgtcgggggaccgaggccgggcaggccgtggcgctgctcggcccgggcctggccgggctggggatcgcggccgccgccgtggccctgggcgtggtgtgggagctggccgaatggtgggggcacacggcggtgaccccggagatcggcgtgggctacacggacaccatcggcgacctcgccgccgatctcgtcggcgccggggtcggcgccgccctcgccgtgtgccgggggcgcacccggtgaccccggcccgccccacggtctccgtggtcgtcccggtgctcgacgacgccgagcacctgcgcgtgtgcctcgcgctgctggccgcccagagccggccggcgctggaggtggtggtggtggacaacggctgcgtggacgactcggcggtgctcgcccgcgccgccggcgcgcgggtggtgcgcgagccgcgccgcggggtcccggccgcggcggccgccggcctggacgccgcggtcggggagctgctggtgcgctgcgacgccgacacgcggatgcccgcggactggctcgaacggatcgtggcccggttcgacgccgaccccgggctcgacgccctcaccgggccggggaccttccacgaccagcccggcctccggggacaggtgcgggcggcgctctacaccggcacgtaccgctggggggcgggcgccgcggtggcggccacccccgtctggggctccaactgcgccctgcgcgccgaggcgtggcaggctgtgcggacccgcgtccaccgcgaacgcggggacgtgcacgatgacctggacctgtccttccagctggccctggccggccgccggatccggttcgatccggacctgcgggtggaggtcgccgggcgcatcttccactccctgcgccagcgggtgcggcagggccggatggcggtcaccaccctgcaggtcaactgggcccgactgtcccccgggcggcgttggctgcgccgggcggcccgggcacacccccggtcccgctgggggcgtggccccgacggtcagtcccgggactga

SEQ ID NO: 2 - DNA, 363 bp, Micrococcus luteus
atgatctacctgctggccctgctgggtgtcatcggctgcatgctgctggtggaccggcgcttcgagctgttcctgtggcatcgcccgctcccggcgctgctggtgctggccgccggggtggcctacttcttcgcctgggacctgtgggggatcgccgaaggcgtgttcctgcaccggcagtcgccctacatgaccggggtgatgctcgccccccagctgcccctggaggaggggttcttcctgctcttcctcagccagatcacgatggtgctgttcaccggggcgctgcgcctgctgcgcggccggcgaggtgacgcccgtgccgcgacggcggccgatccgaccgaccgggggagccggtga

SEQ ID NO: 3 - protein, 120 aa, Micrococcus luteus
MIYLLALLGVIGCMLLVDRRFELFLWHRPLPALLVLAAGVAYFFAWDLWGIAEGVFLHRQSPYMTGVMLAPQLPLEEGFFLLFLSQITMVLFTGALRLLRGRRGDARAATAADPTDRGSR

SEQ ID NO: 4 - DNA, 354 bp, Micrococcus luteus
gtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtcgtgccgatcggcgcggccgtcggccgggacgccgcccccacccggacgatcgctga

SEQ ID NO: 5 - protein, 117 aa, Micrococcus luteus
VTFLDLVLVFVGFALAVLVGAALVGRVRGEHLRAVAATLVALWALTAVFDNVMIAAGLFDYGHELLVGAYVGQAPVEDFAYPLGSALLLPALWLLLTSRRADRRGRRPGRRPHPDDR

SEQ ID NO: 6 - DNA, 885 bp, Micrococcus luteus
atgatccgcaccctcttctgggtgtcccggccggtcagctgggtgaacacggcctacccgttcgccgccgccgcgatcctgaccggggggctgcccgcgtggctggtggtcctgggcgtcgtgttcttcctggtgccctacaacctggccatgtacggcatcaatgacgtgttcgacttcgcctcggacctgcgcaacccccgcaaggggggtgtggagggctccgtgctgggcgaccccgcggtgcgccgccgggtgctggcgtggtcggtgctgctgcccgtgccgttcgtggccgtgctcgcgggctggtccgccgtgcggggcgagtgggccgccgtgctggtgctcgcggtgagcctgttcgcggtggtggcgtactcctgggcggggctgcggttcaaggagcggcccttcctggacgccgccacctccgccacccacttcgtctcccccgcggtctacggcctcgcgctggccggggcgacccccacgcccgccctggcggcgctgctgggggcgttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccgctcgcggcgctgctggccgtgccctacgtggtgaacaccctgcgcttccgccgcatcacggacgccacctcgggcgcggcccaccgcggctggcagctgttccttccgctgaactacgtgaccggcttcctcgtgaccctgctgctgatcgggtgggcgctgacccggggggcggcggcatga

SEQ ID NO: 7 - DNA, 864 bp, Corynebacterium glutamicum
atgatggaaaaaataagactgattctattgtcatctcgccccattagctgggtcaataccgcctacccttttgggctggcatacctattaaatgcaggagagattgactggctgttttggctaggcatcgtgttttttcttatcccgtataacatcgccatgtatggcatcaacgatgtttttgattacgaatctgacatacgtaatccccgcaaaggcggcgtcgagggggccgtgctcccgaaaagttcccacagcacactgttatgggcatcggctatctcaacaattcctttcctagttattcttttcatatttggcacctggatgtcgtctttatggctgacaatctcagtgctagcagtgattgcttattcagcaccgaaattgcgttttaaagaacgcccctttatcgatgctctaacatcttctactcacttcacttcacctgcattaatcggtgcaacgatcactggaacatctccttcagcagcgatgtggatagcactgggatcctttttcttgtggggcatggccagtcagatccttggagcagtacaggatgttaatgcagaccgggaagctaatctgagctcaattgccactgtaattggggcgcgtggagccattcggctatcagtagtactttatttactagctgctgttttagtcactactttgcctaatccggcgtggatcatcgggattgcgattctaacttacgtatttgatgccgcacgattttggaacattacagatgccagttgtgaacaggctaatcgcagttggaaagttttcctgtggctgaactactttgttggtgctgtgataacgatactgttaatagcaattcatcagatataa

SEQ ID NO: 8 - protein, 294 aa, Micrococcus luteus
MIRTLFWVSRPVSWVNTAYPFAAAAILTGGLPAWLVVLGVVFFLVPYNLAMYGINDVFDFASDLRNPRKGGVEGSVLGDPAVRRRVLAWSVLLPVPFVAVLAGWSAVRGEWAAVLVLAVSLFAVVAYSWAGLRFKERPFLDAATSATHFVSPAVYGLALAGATPTPALAALLGAFFLWGMASQMFGAVQDVVPDREGGLASVATVLGARRTVLLAAGLYAAAGLLLLATDPPGPLAALLAVPYVVNTLRFRRITDATSGAAHRGWQLFLPLNYVTGFLVTLLLIGWALTRGAAA

SEQ ID NO: 9 - protein, 287 aa, Corynebacterium glutamicum
MMEKIRLILLSSRPISWVNTAYPFGLAYLLNAGEIDWLFWLGIVFFLIPYNIAMYGINDVFDYESDIRNPRKGGVEGAVLPKSSHSTLLWASAISTIPFLVILFIFGTWMSSLWLTISVLAVIAYSAPKLRFKERPFIDALTSSTHFTSPALIGATITGTSPSAAMWIALGSFFLWGMASQILGAVQDVNADREANLSSIATVIGARGAIRLSVVLYLLAAVLVTTLPNPAWIIGIAILTYVFDAARFWNITDASCEQANRSWKVFLWLNYFVGAVITILLIAIHQI

SEQ ID NO: 10 - DNA, 885 bp, Micrococcus luteus
atgatccgcaccctcttctgggcgtcccggccggtcagctgggtgaacacggcgtacccgttcgccgccgccgcgatcctgaccggggggctgcccgcgtggctggtggtcctgggcgtcgtgttcttcctcgtgccctacaacctggccatgtacggcatcaatgacgtgttcgacttcgcctcggacctgcgcaacccccgcaaggggggcgtggagggctccgtgctgggcgaccccgcggtgcgccgccgggtgctggtgtggtcggtgctgctgcccgtcccgttcgtggccgtgctcgcgggctggtccgccgtgcggggcgagtgggccgccgtgctggtgctggcggtgagcctgttcgcggtggtggcgtactcctgggcggggctgcggttcaaggagcggcccttcctggacgccgcgacctccgccacccacttcgtctcccccgcggtctacggcctcgtgctggccggggcgacccccacgcccgccctggcggcgctgctgggggccttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccccttgcggcgctgctggccgtgccctacgtggtgaacaccctgcgcttccgccgcatcacggacgccacctcgggcgcggcccaccgcggctggcagctgttcctccccctgaactacgtgaccggcttcctcgtgaccctgctgctgatcgggtgggcgctgacccggggggcggcggcatga

SEQ ID NO: 11 - protein, 294 aa, Micrococcus luteus
MIRTLFWASRPVSWVNTAYPFAAAAILTGGLPAWLVVLGVVFFLVPYNLAMYGINDVFDFASDLRNPRKGGVEGSVLGDPAVRRRVLVWSVLLPVPFVAVLAGWSAVRGEWAAVLVLAVSLFAVVAYSWAGLRFKERPFLDAATSATHFVSPAVYGLVLAGATPTPALAALLGAFFLWGMASQMFGAVQDVVPDREGGLASVATVLGARRTVLLAAGLYAAAGLLLLATDPPGPLAALLAVPYVVNTLRFRRITDATSGAAHRGWQLFLPLNYVTGFLVTLLLIGWALTRGAAA

SEQ ID NO: 12 - DNA, 363 bp, Micrococcus luteus
atgatctacctgctggccctgctgggtgtcatcggctgcatgctgctggtggaccggcgcttcgagctgttcctgtggcatcgcccgctcccggcgctgctggtgctggccgccggggtggcctacttcgtcgcctgggacctgtgggggatcgccgaaggcgtgttcctgcaccggcagtcgccctacgtgaccggggtgatgctcgccccccagctgcccctggaggaggggttcttcctgctcttcctcagccagatcacgatggtgctgttcaccggggcgctgcgcctgctgcgcggccggggacgcgacgcccgtgccgcgacgccggccgatccgaccgacggggggagccggtga

SEQ ID NO: 13 - protein, 120 aa, Micrococcus luteus
MIYLLALLGVIGCMLLVDRRFELFLWHRPLPALLVLAAGVAYFVAWDLWGIAEGVFLHRQSPYVTGVMLAPQLPLEEGFFLLFLSQITMVLFTGALRLLRGRGRDARAATPADPTDGGSR

SEQ ID NO: 14 - DNA, 357 bp, Micrococcus luteus
gtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtggtcgtgccggtcggcgcggccctcggccgggacgccgcccccacccggacgatcgctga

SEQ ID NO: 15 - protein, 118 aa, Micrococcus luteus
VTFLDLVLVFVGFALAVLVGAALVGRVRGEHLRAVAATLVALWALTAVFDNVMIAAGLFDYGHELLVGAYVGQAPVEDFAYPLGSALLLPALWLLLTSRGRAGRRGPRPGRRPHPDDR

SEQ ID NO: 16 - DNA, 792 bp, Micrococcus luteus
gtgaccccggcccgccccacggtctccgtggtcgtcccggtgctcgacgacgccgagcacctgcgcgtgtgcctcgcgctgctggccgcccagagccggccggcgctggaggtggtggtggtggacaacggctgcgtggacgactcggcggtgctcgcccgcgccgccggcgcgcgggtggtgcgcgagccgcgccgcggggtcccggccgcggcggccgccggcctggacgccgcggtcggggagctgctggtgcgctgcgacgccgacacgcggatgcccgcggactggctcgaacggatcgtggcccggttcgacgccgaccccgggctcgacgccctcaccgggccggggaccttccacgaccagcccggcctccggggacaggtgcgggcggcgctctacaccggcacgtaccgctggggggcgggcgccgcggtggcggccacccccgtctggggctccaactgcgccctgcgcgccgaggcgtggcaggctgtgcggacccgcgtccaccgcgaacgcggggacgtgcacgatgacctggacctgtccttccagctggccctggccggccgccggatccggttcgatccggacctgcgggtggaggtcgccgggcgcatcttccactccctgcgccagcgggtgcggcagggccggatggcggtcaccaccctgcaggtcaactgggcccgactgtcccccgggcggcgttggctgcgccgggcggcccgggcacacccccggtcccgctgggggcgtggccccgacggtcagtcccgggactga

SEQ ID NO: 17 - protein, 263 aa, Micrococcus luteus
VTPARPTVSVVVPVLDDAEHLRVCLALLAAQSRPALEVVVVDNGCVDDSAVLARAAGARVVREPRRGVPAAAAAGLDAAVGELLVRCDADTRMPADWLERIVARFDADPGLDALTGPGTFHDQPGLRGQVRAALYTGTYRWGAGAAVAATPVWGSNCALRAEAWQAVRTRVHRERGDVHDDLDLSFQLALAGRRIRFDPDLRVEVAGRIFHSLRQRVRQGRMAVTTLQVNWARLSPGRRWLRRAARAHPRSRWGRGPDGQSRD

SEQ ID NO: 18 - DNA, 1077 bp, Micrococcus luteus
atgggtgaagcgaggacgggcggcgaggccgcgctctccggggtgaccgccgagctggacgccgcgctccgacacgccgcggcccaggcgcccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgactgcggccggcgagctcgaggacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgccgcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctga

SEQ ID NO: 19 - protein, 358 aa, Micrococcus luteus
MGEARTGGEAALSGVTAELDAALRHAAAQAPGSAAFAELLDSLHVHVGAGKLIRPRLVELGWRLATADPVPPSGRAAVDRLGAAFELLHTALLVHDDVIDRDVLRRGQPAVHASARHRLEARGVPAADAAHAGVAVALIAGDVLLTQAFRLAATCAADTARAAEAAAVVFDAAAVTAAGELEDVLLGLSRHTGEEPDPDRILAMQRLKTAHYTVGAPLRAGALLAGADPDLARAMGEAGADLGAAYQVIDDVLGVFGDPGETGKSADGDLREGKATVLTAHGRRIPAVRALLDAGPATPADIEAARRALEAAGAREHALDVAAELTVRARERIAALPLDETVRAEFADACHAVLTRRS

SEQ ID NO: 20 - DNA, 897 bp, Micrococcus luteus
atggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggccgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtccctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatga

SEQ ID NO: 21 - protein, 298 aa, Micrococcus luteus
MAAPTPSPAALYTRTAHTAAAQVIRRYSTSFSWACRTLPRQARQDVATIYAMVRVADEVVDGVAVAAGLDEAGVRAALDDYERACEAAMASGFATDPVLHAFADVARRHGITPELTRPFFASMRADLGIREHGAESLDAYIHGSAEVVGLMCLQVFLSLPGTRARTPGQRQELRAQASRLGAAFQKVNFLRDLAADHHELGRTYLPGAAPGVLTEARKAELVAEVRADLDAALPGIRVLDPGAGRAVALAHGLFAALVDRIEATPAAELAHRRVRVPDHQKARIAARVLARGRRGGRR

SEQ ID NO: 22 - DNA, 1700 bp, Micrococcus luteus
atgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtccgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtcgcggcccgcttccaggacccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtggaggtcgcggaccgtcccgcccccgccggccgcctcggacgcctggccgcccgcctgcccaggccgggagcagcccgcggggacgagggccgacgtcgccgcccgggccgggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacacctgctgttcacggcggactgggaggacaacttcgggcgcatcgagcggggggaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggccagggctggcgggtggaccgcgccggggacgcccaggtggaggccgtggcggaccgggccctcgatcagctggcccgctgggccgggatccccgacctggccgagcgcatcgtggtgcggcgcacctacgggcccggtgacttcgccgcggacgtgcacgcctggcggggttcgctgctgggccccgggcacacgctggcgcagtcggccatgttccgcccctcggtgcgggacgcggacgtggccggcctgatgtacgcgggctcctcggtgcgcccgggaatcggggtgcccatgtgcctgatctccgccgaagtggtccgggacgaactgcgccacgacgcgcgcagggcccggcccgcgggccccggggggagcggcacatga

SEQ ID NO: 23 - protein, 566 aa, Micrococcus luteus
MSARDTALGPRTVVVGGGFAGLATAGLLARDGHRVTLLERGAVLGGRAGRWSEAGFTFDTGPSWYLMPEVIDRWFRLMGTSAAERLDLRRLDPGYRVYFEGHLHEPPVDVRTGHAETLFESLEPGAGRRLRAYLDSASRIYGLAKEHFLYTDFRRPAALAHPDVLRALPALGPQLLGGLRSHVAARFQDPRLRQILGYPAVFLGTSPDRAPAMYHLMSHLDLADGVQYPLGGFAALVDAMAEVVREAGVEIRTGVEATAVEVADRPAPAGRLGRLAARLPRPGAARGDEGRRRRPGRVTGVAWRSDDGAAGRLDADVVVAAADLHHVQTRLLPPGRRVAESTWDRRDPGPSGVLVCVGVRGSLPQLAHHTLLFTADWEDNFGRIERGEDLAADTSIYVSRTSATDPGVAPEGDENLFILVPAPAEPGWGRGGIRVRDGQGWRVDRAGDAQVEAVADRALDQLARWAGIPDLAERIVVRRTYGPGDFAADVHAWRGSLLGPGHTLAQSAMFRPSVRDADVAGLMYAGSSVRPGIGVPMCLISAEVVRDELRHDARRARPAGPGGSGT

SEQ ID NO: 24 - DNA, 609 bp, Micrococcus luteus
gtgccgatcggcgcggccgtcggccgggacgccgcccccacccggacgatcgctgacatgctgccgttgatccccgcagacctgctgcgcgcgctcggcctgatcctcgtcccggtcgcggcggtgcacgccggatggccgtccgcggcggcgatgctgctcgtgttcggctcccagtggctcacccgctggctcgccccgggcggcgccctggactgggccgcgcaggcggtcctgctgctggccgggtggctgagcgtcatcggcctctacccgcgggtgccgtggctggacctgctcgtgcacgccgccgcctccgccgtggtcgcctgtctgacggcactggtggtgggggcgtggctccggcgtcgggggaccgaggccgggcaggccgtggcgctgctcggcccgggcctggccgggctggggatcgcggccgccgccgtggccctgggcgtggtgtgggagctggccgaatggtgggggcacacggcggtgaccccggagatcggcgtgggctacacggacaccatcggcgacctcgccgccgatctcgtcggcgccggggtcggcgccgccctcgccgtgtgccgggggcgcacccggtga

SEQ ID NO: 25 - protein, 202 aa, Micrococcus luteus
VPIGAAVGRDAAPTRTIADMLPLIPADLLRALGLILVPVAAVHAGWPSAAAMLLVFGSQWLTRWLAPGGALDWAAQAVLLLAGWLSVIGLYPRVPWLDLLVHAAASAVVACLTALVVGAWLRRRGTEAGQAVALLGPGLAGLGIAAAAVALGVVWELAEWWGHTAVTPEIGVGYTDTIGDLAADLVGAGVGAALAVCRGRTR

SEQ ID NO: 26 - DNA, 5379 bp, Micrococcus luteus
atgggtgaagcgaggacgggcggcgaggccgcgctctccggggtgaccgccgagctggacgccgcgctccgacatgccgcggcccaggcacccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgaccgcggccggcgagctcgaagacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgcctcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctgagatggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggctgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtcgctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtctgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtggcggcccgcttccaggatccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtcgaggtggtggaccgtcccgcccccgccggccgcctcggacgcctggccgcccgcctgcccaggccgggagcagcccgcggggacgagggccgacgtcgccgcccgggccaggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggagaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggcgagggctggcgggtggaccgcgccggggacgcccaggtggaggccgtggcggaccgggccctcgaccagctggcccgctgggccgggatcccggacctggccgagcgcatcgtggtgcggcgcacctacgggcccggtgacttcgccgcggacgtgcacgcctggcggggttcgctgctgggccccgggcacacgctggcgcagtcggccatgttccgtccctcggtgcgggacgcggacgtggccggcctgatgtacgcgggctcctcggtgcgcccgggcatcggggtgcccatgtgtctgatctccgccgaagtggtccgggacgaactgcgccacgacgcgcgcagggcccggcccgcgggccccggggggagcggcacatgatccgcaccctcttctgggcgtcccggccggtcagctgggtgaacacggcgtacccgttcgccgccgccgcgatcctgaccggggggctgcccgcgtggctggtggtcctgggcgtcgtgttcttcctcgtgccctacaacctggccatgtacggcatcaatgacgtgttcgacttcgcctcggacctgcgcaacccccgcaaggggggcgtggagggctccgtgctgggcgaccccgcggtgcgccgccgggtgctggtgtggtcggtgctgctgcccgtcccgttcgtggccgtgctcgcgggctggtccgccgtgcggggcgagtgggccgccgtgctggtgctggcggtgagcctgttcgcggtggtggcgtactcctgggcggggctgcggttcaaggagcggcccttcctggacgccgcgacctccgccacccacttcgtctcccccgcggtctacggcctcgtgctggccggggcgacccccacgcccgccctggcggcgctgctgggggccttcttcctgtggggcatggcctcgcagatgttcggggcggtgcaggacgtggtgccggaccgggaggggggcctggcctcggtggccaccgtgctgggcgctcggcgcaccgtcctgctcgccgccggcctgtacgcggcggcgggcctgctgctgctggccaccgacccgccgggcccccttgcggcgctgctggccgtgccctacgtggtgaacaccctgcgcttccgccgcatcacggacgccacctcgggcgcggcccaccgcggctggcagctgttcctccccctgaactacgtgaccggcttcctcgtgaccctgctgctgatcgggtgggcgctgacccggggggcggcggcatgatctacctgctggccctgctgggtgtcatcggctgcatgctgctggtggaccggcgcttcgagctgttcctgtggcatcgcccgctcccggcgctgctggtgctggccgccggggtggcctacttcgtcgcctgggacctgtgggggatcgccgaaggcgtgttcctgcaccggcagtcgccctacgtgaccggggtgatgctcgccccccagctgcccctggaggaggggttcttcctgctcttcctcagccagatcacgatggtgctgttcaccggggcgctgcgcctgctgcgcggccggggacgcgacgcccgtgccgcgacgccggccgatccgaccgacggggggagccggtgaccttcctcgacctcgtcctcgtcttcgtgggcttcgccctggccgtgctcgtgggcgccgccctcgtcggccgcgtgcggggcgagcacctgcgggccgtggcggccaccctggtggccctgtgggccctcacggcggtcttcgacaacgtgatgatcgccgcggggctcttcgactacggccatgagctgctggtgggtgcctacgtgggccaggcgcccgtggaggacttcgcctacccgctcggctccgccctgctgctgccggcgctctggctgctgctgacgagccgtggtcgtgccggtcggcgcggccctcggccgggacgccgcccccacccggacgatcgctgagcggccgcaaaaaaatcactagtgcggccgcctgcaggtcgaccatatgggagagctcccaacgcgttggatgcatagcttgagtattctatagtgtcacctaaatagctggcg

SEQ ID NO: 27 - DNA, 1077 bp, Micrococcus luteus
atgggtgaagcgaggacgggcggcgaggccgcgctctccggggtgaccgccgagctggacgccgcgctccgacatgccgcggcccaggcacccggatccgccgccttcgccgagctgctcgactcgctccacgtccatgtgggcgccggcaagctcatccgcccccgtctcgtcgagctcggctggcgcctggcgaccgccgacccggtccctccgtccggccgcgctgccgtcgaccgactcggggccgccttcgaactgctgcacaccgcgctgctcgtccacgacgacgtcatcgatcgggacgtgctgcggcgcggccagcccgccgtgcacgcctccgcccggcaccgcctcgaggcccgcggggtgcccgccgcggacgccgcccacgccggggtcgccgtcgccctcatcgcgggggacgtcctgctcacccaggcgttccggctcgccgccacctgtgccgccgacaccgcccgggccgccgaggccgccgccgtcgtcttcgacgccgccgccgtgaccgcggccggcgagctcgaagacgtgctcctggggctgtcccgccacaccggtgaggagcccgatcccgaccgcatcctcgccatgcaacggctcaagacggcgcactacacggtcggcgcgcccctgcgcgccggcgccctcctggccggggcggatcccgacctcgcccgggcgatgggcgaggccggcgccgacctcggcgccgcctaccaggtgatcgacgacgtcctcggcgtgttcggcgatcccggggagaccggcaagtccgccgacggcgacctgcgcgagggcaaggccaccgtgctcaccgcccacggccgcctcatccccgccgtccgcgccctgctcgacgcgggcccggccacccccgcggacatcgaggccgcccgccgcgccctcgaggcggccggtgcccgggagcacgccctcgacgtcgccgccgagctcaccgtccgcgcccgcgagcgcatcgcggccctgcccctggacgagacggtccgggcggagttcgccgacgcctgccacgccgtgctgacccggaggtcctga

SEQ ID NO: 28 - protein, 358 aa, Micrococcus luteus
MGEARTGGEAALSGVTAELDAALRHAAAQAPGSAAFAELLDSLHVHVGAGKLIRPRLVELGWRLATADPVPPSGRAAVDRLGAAFELLHTALLVHDDVIDRDVLRRGQPAVHASARHRLEARGVPAADAAHAGVAVALIAGDVLLTQAFRLAATCAADTARAAEAAAVVFDAAAVTAAGELEDVLLGLSRHTGEEPDPDRILAMQRLKTAHYTVGAPLRAGALLAGADPDLARAMGEAGADLGAAYQVIDDVLGVFGDPGETGKSADGDLREGKATVLTAHGRLIPAVRALLDAGPATPADIEAARRALEAAGAREHALDVAAELTVRARERIAALPLDETVRAEFADACHAVLTRRS

SEQ ID NO: 29 - DNA, 897 bp, Micrococcus luteus
atggccgcgcccaccccgagccctgccgcgctgtacacgcggacggcccacaccgcagcggcccaggtgatccgccgctactccacgtccttctcctgggcctgccgcaccctgccccggcaggcacgccaggacgtggccacgatctacgccatggtccgcgtcgccgacgaggtggtcgacggcgtcgcggtggccgccgggctcgacgaggccggggtccgcgccgccctggacgactacgagcgggcgtgtgaggctgcgatggcgtcgggcttcgccaccgacccggtcctgcacgccttcgccgacgtggcccgtcgccacggcatcaccccggagctgacccgtcccttcttcgcctccatgcgcgcggacctggggatccgcgagcacggcgccgagtcgctggacgcctacatccacggctcggccgaggtggtggggctgatgtgcctgcaggtcttcctctccctccccggcacgcgggcccggaccccgggccagcggcaggagctgcgcgcgcaggcctcccggctgggggcggcgttccagaaggtcaacttcctcagggacctggccgcggaccaccacgagctgggccgcacctacctgcccggtgccgcaccgggcgtgctcaccgaggcccgcaaggccgagctcgtggccgaggtccgcgccgacctcgacgccgccctgcccggcatccgtgtcctggaccccggggccgggcgcgccgtggccctggcgcacggactgttcgcggccctggtggaccggatcgaggcgaccccggcggccgagctggcccaccgccgtgtccgggtgccggaccatcagaaggcccggatcgccgcccgcgtcctggcacggggccgccggggaggccgccgatga

SEQ ID NO: 30 - protein, 298 aa, Micrococcus luteus; identical to SEQ ID NO: 21

SEQ ID NO: 31 - DNA, 1701 bp, Micrococcus luteus
atgagcgcccgggacaccgctctcggcccgcgcaccgtggtggtgggcggcggtttcgccggactggccacggcgggcctgttggcccgcgacgggcaccgggtgacgctgctggagcgcggcgccgtcctgggcggccgtgccggacgctggtctgaggcggggttcaccttcgataccgggccctcctggtacctgatgcccgaggtgatcgaccgctggttccgcctcatggggacctccgccgccgaacggctggacctgcgccgtctggaccccggctaccgggtgtacttcgaggggcacctccacgagccccccgtggacgtgcgcaccggccacgcggagacgctgttcgagtccctcgagcccggcgccgggcgccggctgcgggcctacctcgactccgcgtcccggatctacgggctcgccaaggagcacttcctctacacggacttccgccggccggccgccctggcccacccggacgtcctgcgcgccctgccggccctcgggccccagctgctggggggcctgcgctcccacgtggcggcccgcttccaggatccccggctgcgccagatcctgggctacccggcggtcttcctcggcacgtcccccgaccgtgcccccgccatgtaccacctgatgtcccatctggacctcgccgacggcgtgcagtaccccctcggcgggttcgcggccctcgtggacgccatggcggaggtcgtgcgcgaggccggcgtggagatccgcaccggggtcgaggcgaccgccgtcgaggtggtggaccgtcccgcccccgccggccgcctcggacgcctggccgcccgcctgcccaggccgggagcagcccgcggggacgagggccgacgtcgccgcccgggccaggtgaccggcgtcgcctggcggtccgacgacggcgccgcgggacgcctcgacgccgatgtggtggtggccgccgcggacctgcaccacgtgcagacccgtctgctgcctcccggccggcgcgtcgcggagtccacgtgggaccggcgcgaccccggcccctccggcgtgctcgtgtgcgtgggggtgcgcggatccctgccccagctggcccatcacaccctgctgttcacggcggactgggaggacaacttcgggcgcatcgagcggggagaggacctcgccgcggacacgtcgatctacgtctcgcgcacctccgccacggacccgggcgtggccccggagggcgacgagaacctcttcatcctcgtcccggcccccgccgagccggggtgggggcgcggcggcatccgggtccgtgacggcgagggctggcgggtggaccgcgccggggacgcccaggtggaggccgtggcggaccgggccctcgaccagctggcccgctgggccgggatcccggacctggccgagcgcatcgtggtgcggcgcacctacgggcccggtgacttcgccgcggacgtgcacgcctggcggggttcgctgctgggccccgggcacacgctggcgcagtcggccatgttccgtccctcggtgcgggacgcggacgtggccggcctgatgtacgcgggctcctcggtgcgcccgggcatcggggtgcccatgtgtctgatctccgccgaagtggtccgggacgaactgcgccacgacgcgcgcagggcccggcccgcgggccccggggggagcggcacatga

SEQ ID NO: 32 - protein, 566 aa, Micrococcus luteus
MSARDTALGPRTVVVGGGFAGLATAGLLARDGHRVTLLERGAVLGGRAGRWSEAGFTFDTGPSWYLMPEVIDRWFRLMGTSAAERLDLRRLDPGYRVYFEGHLHEPPVDVRTGHAETLFESLEPGAGRRLRAYLDSASRIYGLAKEHFLYTDFRRPAALAHPDVLRALPALGPQLLGGLRSHVAARFQDPRLRQILGYPAVFLGTSPDRAPAMYHLMSHLDLADGVQYPLGGFAALVDAMAEVVREAGVEIRTGVEATAVEVVDRPAPAGRLGRLAARLPRPGAARGDEGRRRRPGQVTGVAWRSDDGAAGRLDADVVVAAADLHHVQTRLLPPGRRVAESTWDRRDPGPSGVLVCVGVRGSLPQLAHHTLLFTADWEDNFGRIERGEDLAADTSIYVSRTSATDPGVAPEGDENLFILVPAPAEPGWGRGGIRVRDGEGWRVDRAGDAQVEAVADRALDQLARWAGIPDLAERIVVRRTYGPGDFAADVHAWRGSLLGPGHTLAQSAMFRPSVRDADVAGLMYAGSSVRPGIGVPMCLISAEVVRDELRHDARRARPAGPGGSGT

(19) United States
(12) Patent Application Publication
(10) Pub. No.: US 2013/0078203 A1
(43) Pub. Date: Mar. 28, 2013

(54) CAROTENOID SUNSCREEN

(71) Applicant: Audun Goksøyr, Fornebu (NO)

(72) Inventor: Audun Goksøyr, Fornebu (NO)

(73) Assignee: Promar AS, Fornebu (NO)

(21) Appl. No.: 13/701,249

(22) PCT Filed: Jun. 1, 2011
(86) PCT No.: PCT/EP2011/059161

§ 371 Date: Nov. 30, 2012

(30) Foreign Application Priority Data
Jun. 2, 2010 (GB) 1009271.6

(51) Int. Cl. A61K 8/60; A61Q 17/04; A61Q 5/00; A61K 8/362

(52) U.S. Cl. CPC A61K 8/602; A61K 8/362; A61Q 17/04; A61Q 5/00
USPC 424/59; 424/70.9

(57) ABSTRACT

The invention concerns methods of treating or preventing the effects of irradiation in a human or non-human animal using sarcinaxanthin and related compounds (particularly its glycosides) as well as photoprotective compositions and their use to prepare photoprotective or photoprotected products.

The present invention relates to compositions comprising the carotenoid sarcinaxanthin and related compounds. Preferably the compositions are pharmaceutical or cosmetic compositions, particularly compositions with photoprotective properties, such as sunscreens for preventing damage resulting from exposure of body coverings or surfaces such as skin and hair to the UV- and visible range of the solar spectrum.

Sunlight is composed of a continuous spectrum of electromagnetic radiation that is divided into three main regions of wavelengths: ultraviolet (UV), visible, and infrared. UV radiation comprises the wavelengths from 200 to 400 nm, while visible light ranges from 400 to 700 nm. The ultraviolet spectrum is further divided into three sections, each of which has distinct biological effects: UVA (320-400 nm), UVB (280-320 nm), and UVC (200-280 nm).

The damaging effects of sunlight on skin are well documented, and the multiple deleterious effects include burns, premature aging and wrinkling of the skin (dermatoheliosis), development of pre-malignant lesions (solar keratoses) and various malignant tumours.

While the UVC rays are effectively blocked from reaching the Earth's surface by the stratospheric ozone layer, UVA and UVB radiation both reach the Earth's surface in amounts sufficient to have important biological consequences to the skin and eyes. Of the UV radiation that reaches the surface of the earth, 90-99% is comprised of UVA and 1-10% is comprised of UVB. The damaging effects of UVB have been widely documented. The short term effects of these high intensity rays include erythema and burns. In the longer term the risk of skin cancer is significant as UV radiation from 245 to 290 nm is absorbed maximally by DNA, and is able to directly induce mutagenic photoproducts or lesions in DNA among adjacent pyrimidines in the form of dimers.

UVA rays are not directly absorbed by DNA, but can have indirect harmful effects by forming radical oxygen species that can react with cellular proteins and DNA. The UVA rays are lower in intensity; they penetrate below the skin surface and cause long-term damage such as premature wrinkling and photoaging, and are believed to be carcinogenic. Skin cancer is the most common type of cancer, in the US about 800 000 cases occur each year. Most skin cancers are either basal cell or squamous type and tend to grow and spread slowly. Malignant melanoma is a much more serious form of skin cancer and is now increasing by about 4% per year.

The exact wavelength of radiation in the solar spectrum which induces melanoma is not known, but the limited data that are available suggest that the UVR spectrum is most important, particularly UVB but possibly also UVA and visible blue light. With the growing awareness that UVA damage exacerbates the risk of melanoma and other tumours, the need for broad spectrum protection has become obvious. The classical means of measuring sunscreen efficiency is the sun protection factor (SPF) number, which is defined as the prolonged exposure to UVB rays the skin can endure before getting burned, compared to untreated skin. Several studies speak of the potentially dangerous false sense of security the SPF factor gives with regards to damage induced by UVA and visible blue light.

In view of their convenience of use, sunscreens have assumed a major component of protection against sun rays. Sunscreens work by absorbing, reflecting or scattering the sunrays, and thereby either shielding the skin from the sun's rays or transforming the light energy to a harmless energy form. Sun protecting agents can roughly be divided into chemical and physical filters. The physical sunscreens are inorganic microparticles that act as broad spectrum photoprotectors by reflecting or scattering the sunrays. Extensively used physical barriers include zinc oxide and titanium dioxide. They are known to provide good photoprotection but are less appealing cosmetically; they are not absorbed by the skin and tend to stay as a white layer on the skin surface.

Chemical sunscreens are absorbed by the skin, and exert their sunscreen activity by absorbing the rays emitted by the sun and re-emitting this light energy as vibrational energy (heat). Common chemical sunscreen agents include PABA (para-amino benzoic acid) and its derivatives, cinnamates, salicylates, anthranilates, camphor derivatives, benzimidazole, triazones, octocrylene, urocanic acid, bisimidazylate and anisotriazine.

Consumer safety is a major concern with regards to sunscreen compounds. Available research establishes that some sunscreen compounds are potentially photo allergenic; for example PABAs, that are known to induce photo allergenic reactions in 1-2% of the population (Kimbrough, 1997, J. Chem. Ed., 74(1), p 51-53). Although generally regarded as good photo-protectors, the safety of the physical sunscreen has also been discussed, as in vitro studies with human fibroblasts has shown formation of hydroxyl radicals upon the combination of sun exposure and titanium dioxide, which led to strand breakage in the DNA (Dunforda et al, 1997, FEBS Lett., 418, p 87-90). In addition, all of these chemicals photo decompose into unknown compounds and the long-range safety effects have not been studied.

There is particularly a need for a good means for rating UVA protection, as no such standard exist today. Despite increasing awareness of the importance of broad spectrum protection, studies show that commercially available sunscreens claiming to have good UVA protection do not protect sufficiently against UVA rays (Haywood et al, 2003, J. Invest. Derm., 121(4), p 862). Particularly, in the longer wavelength UVA radiation (370-400 nm) the available sun filters provide poor protection and particularly poor or no protection against wavelengths above 400 nm.

Most of the commercially available UV- and sun protecting compounds in skin creams are synthetic, and the search for natural compounds with equal or greater efficiency is becoming more significant because of the consumer's preference for natural products.

The UV-absorbing properties of various organisms and natural extracts have been studied among higher plants, corals, cyanobacteria and phytoplankton, but commercialisation of natural sunscreen compounds is still limited. There remains a need for naturally derived sun-absorbing or sunscreen agents that are efficient filters of sun in the UV- and visible range of the solar spectrum.

Surprisingly it has been found that sarcinaxanthin and related compounds are effective UV and visible light filters (particularly for use on the skin of animals, especially humans), are antioxidants, have a golden yellow colour, are oil soluble and stable. Compounds of particular interest are sarcinaxanthin and its glycosides, specifically its mono- and di-glucosides (and mono- and di-mannosides).

Sarcinaxanthin is a γ-cyclic C₅₀ carotenoid which was first described in 1941 by Takeda and Ohta (Hoppe-Seyler's Zeitschrif für Physiologische Chemie, Vol. 268, Issue 3-4, pI-IV). Sarcinaxanthin is a carotenoid found in marine microorganisms such as Micrococcus luteus which are found throughout nature, e.g. in soil, water and skin. Sarcinaxanthin has also been identified in Cellulomonas biazotea (Weeks et al., J. Bacteriol., 1980, 141(3), p 1272-1278) and in a coryneform organism (Hodgkiss et al., 1954, J. Gen. Microbiol., 11, p 488-4150).

The present inventors have found that sarcinaxanthin and related compounds have particularly useful properties as sunscreens, particularly when applied to living organisms.

Whilst other carotenoids have been identified as having utility as sunscreens, see e.g. WO2006/077433 and U.S. Pat. No. 6,787,147, sarcinaxanthin and its related compounds have not previously been identified as having any utility as sun-absorbing compounds. Indeed, no C₅₀ carotenoids have previously been identified or suggested for this purpose.

Sarcinaxanthin has surprisingly been found to be useful in absorbing irradiation, particularly in the previously overlooked blue light range and thus has utility in applications reliant on sun-absorbing properties, e.g. as sunscreens, particularly in view of its stability.

In a first aspect, the present invention provides a composition comprising a carotenoid which has the formula:

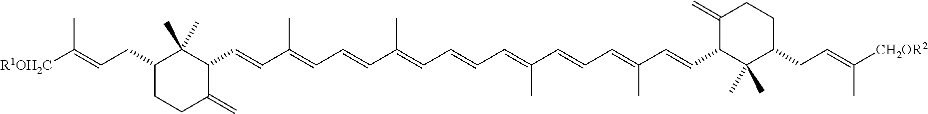

wherein R¹ and R², which may be the same or different, are each a hydrogen atom or a saccharide, preferably a monosaccharide such as mannose or glucose (preferably glucose), or a pharmaceutically acceptable derivative or salt thereof.

In particularly preferred aspects R¹ and R² are both hydrogen atoms or one or both of R¹ and R² are glucose or mannose moieties.

Preferably the above described family does not encompass naturally occurring carotenoids, other than specifically mentioned carotenoids described herein in accordance with the invention, e.g. sarcinaxanthin and its glycosides and preferably also their naturally occurring derivatives.

Especially preferably the carotenoid is: 2,2′-bis(4-hydroxy-3-methyl-2-butenyl)]-γ-γ-carotene (preferably 2R,6S,2′R,6′S) or its glycosides.

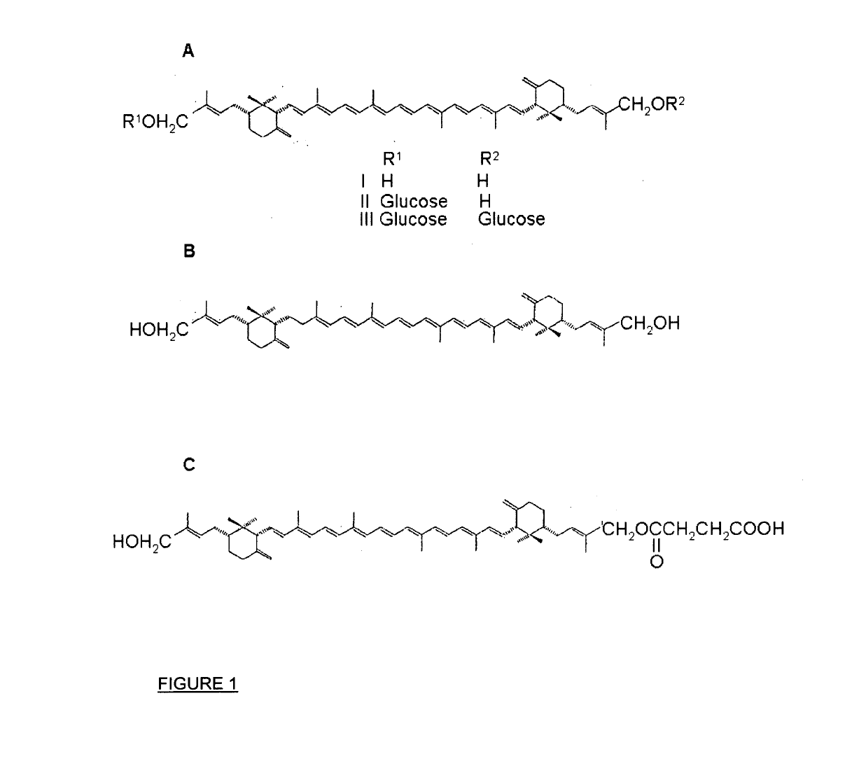

Especially preferably said compound is sarcinaxanthin or its mono- or di-glucoside which compounds have the structures shown in FIG. 1.

By “pharmaceutically acceptable” or “physiologically acceptable” is meant that the ingredient must be compatible with other ingredients in the composition as well as physiologically acceptable to the recipient. Pharmaceutically acceptable derivatives (which have the same or similar functional properties to the compounds described above), include isomers ranging from all trans (native) to a mixture of cis-trans to all cis isomers and includes optical isomers such as the 2R,6R,2′R,6′R. Preferably the isomers are 2R,6S,2′R,6′S or 2R,6R,2′R,6′R.

Derivatives further include molecules which have been modified by e.g. modification of the hydrocarbon backbone, e.g. by substitution with one or more alkyl groups or modification of either or both of the cyclic groups (e.g. as described hereinbefore), providing such modifications do not alter the functional properties of the compounds as described herein. For example, derivatives extend to esters, e.g. the carotenoids may be esterified with fatty acids. Preferred esters are as described in US2005/0096477 which describes astaxanthin esters and is hereby incorporated by reference, particularly in relation to the esters which are generated. Sarcinaxanthin may be similarly modified. Preferably the ester is sarcinaxanthin succinate or disuccinate.

Derivatives include molecules in which one or more double bonds within the hydrocarbon backbone may be hydrogenated. Preferred derivatives in this regard are 7,8-dihydrosarcinaxanthin (Λ_{MAX} 398, 421, 446 nm) which has been identified in M. luteus (Norgard et al, 1970, Acta Chem. Scand., 24, p 1460-1462 and Arpin et al, 1973, Acta Chem. Scand., 27, p 2321-2334) and 7,8,7′,8′-tetrahydrosarcinaxathin (and their glucosides).

Derivatives may also be generated to modify compounds of the invention for their use in cosmetic and pharmaceutical applications, e.g. by the addition of targeting or functional groups, e.g. to improve lipophilicity, aid cellular transport, solubility and/or stability. Thus oligosaccharides, fatty acids, fatty alcohols, amino acids, peptides or proteins may be conjugated to the aforementioned compounds.

Derivatives may be in the form of “pro-drugs” such that the added component may be removed by cleavage once administered, e.g. by cleavage of a substituent added through esterification which may be removed by the action of esterases.

Derivatives which retain functional activity may be tested to establish if they retain the desired properties by the test described herein e.g. to determine photoprotective properties.

The active ingredient for administration may be appropriately modified for use in a pharmaceutical composition. For example the compounds used in accordance with the invention may be stabilized against degradation by the use of derivatives as described above.

The active ingredient may also be stabilized in the compositions for example by the use of appropriate additives such as salts or non-electrolytes, acetate, SDS, EDTA, citrate or acetate buffers, mannitol, glycine, HSA or polysorbate.

Pharmaceutically acceptable salts are preferably acid addition salts with physiologically acceptable organic or inorganic acids. Suitable acids include, for example, hydrochloric, hydrobromic, sulphuric, phosphoric, acetic, lactic, citric, tartaric, succinic, maleic, fumaric and ascorbic acids. Hydrophobic salts may also conveniently be produced by for example precipitation. Appropriate salts include for example acetate, bromide, chloride, citrate, hydrochloride, maleate, mesylate, nitrate, phosphate, sulfate, tartrate, oleate, stearate, tosylate, calcium, meglumine, potassium and sodium salts. Procedures for salt formation are conventional in the art.

Preferably the compounds used in compositions and uses of the invention are obtained or derived from naturally occurring sources. They may however be generated entirely or partially synthetically (e.g. from commercially available carotenoids such as lycopene, or derivatized after purification). Preferably the compounds are isolated from natural sources, preferably from M. luteus. In a preferred alternative the compounds are produced as described in the Examples. Further methods for production of the compounds are as described in the international application PCT/EP2011/059159 (filed on 1 Jun. 2011) claiming priority from GB patent application no. 1009269.0 (filed on 2 Jun. 2010) whose subject matter is hereby incorporated by reference.

Compounds of the invention may be isolated from natural sources or isolated from natural sources which have been modified to allow production of the carotenoids used in the invention, e.g. by transformation of microbiological organisms to produce the required synthetic enzymes and isolation of the compounds from those organisms.

Conveniently such compounds are isolated by techniques known in the art such as by extraction using organic solvents or by lipid precipitation or HPLC (Zapata et al., 2000, MEPS, 195, p 29-45).

Compounds for use in compositions of the invention may also be isolated in accordance with the protocols described in the Examples.

Carotenoids used in accordance with the invention may be generated synthetically based, for example, on a synthetic carbon skeleton. Such skeletons may be generated using techniques known in the art, such as Witting type reactions, Grignard and Nef reactions, enol ether condensations, Reformatsky reactions, Robinson's Mannic base synthesis, reductive or oxidative dimerisations and Wurtz reactions (see e.g. Haugan, Dr. Ing. thesis, University of Trondheim, NTH, 1994, from p 155 and Mayer & Isler, 1971, in “Carotenoids”, Ed. Isler, Birkhäuser, Basel, p 325).

The carbon skeleton may then be modified accordingly to generate the carotenoid of interest using techniques known in the art.

The synthesis of sarcinaxanthin is described for example in Lanz et al., Helvetica Chimica Acta, 2004, Vol. 80(3), p 804-827 and Férézou and Julia, Tetrahedron, 1990, Vol. 46(2), p 475-486.

Derivatives of these synthetically prepared carotenoids may be made as described above using techniques known in the art. Glycosides may be generated by co-expression of the crtX gene in E. coli expressing sarcinaxanthin (see Example 2) or glycosylation may be achieved by well known non-enzymatic glycation techniques.

Compounds which are isolated or synthesized are preferably substantially free of any contaminating components derived from the source material or materials used in the isolation procedure. Especially preferably the compound is purified to a degree of purity of more than 50 or 60%, e.g. >70, 80 or 90%, preferably more than 95 or 99% purity as assessed w/w (dry weight). Such purity levels correspond to the specific compound of interest, but including its isomers and optionally its degradation products. Where appropriate, enriched preparations may be used which have lower purity, e.g. contain more than 1, 2, 5 or 10% of the compound of interest, e.g. more than 20 or 30%.

Conveniently the level of purity may be assessed by analysis, e.g. using UV/visible spectrophotometry, HPLC analysis or mass spectrometry. Synthetically generated or modified compounds should be similarly free from contaminating components.

The carotenoid compound may be present in said compositions as the sole active ingredient or may be combined with other ingredients, particularly other active ingredients, e.g. to increase the range over which light protection may be offered and/or to change the physical or chemical characteristics of the product or to make it appealing to the consumer. Thus for example one or more additional sunscreen compounds may be included in the composition or co-administered with the composition. Chemical or physical sunscreen agents may be used, e.g. as described hereinbefore which are able to absorb/quench radiation, particularly solar radiation, particularly in the UVB and shorter UVA range or infrared region of the spectrum. Compounds which may be used include UVB/UVA2 filters (which filter in the range 290-340 nm) such as octyl methoxy-cinnamate, oxybenzone, octyl salicylate, homosalate, octocrylene, padimate 0, menthyl anthranilate and 2-phenylbenzimadazole-5-sulfonic acid. UVA1 filters (filtering in the range 340-400 nm) include avobenzone, zinc oxide and titanium dioxide. Preferably however, compounds are used which are found naturally, e.g. other carotenoids, (e.g. as described herein), mycosporine-like amino acids or scytonemin.

Carotenoids as described herein may be used in combination. Thus for example preferred compositions in accordance with the invention may include two or more carotenoids as described herein, e.g. two or more compounds selected from sarcinaxanthin, its glycosides or pharmaceutical derivatives thereof, e.g. sarcinaxanthin and sarcinaxanthin monoglucoside and/or sarcinaxanthin diglucoside and/or sarcinaxanthin succinate and/or 7,8-dihydrosarcinaxanthin.

The composition of the invention may be used in various biological and non-biological applications. Thus the compositions may be used in any non-biological material in which photoprotective (or colouring) properties are desirable, e.g. in plastics, paints, waxes, windows (of buildings or vehicles), solar panels, windshields, stains or lacquers, glass, contact lenses, synthetic lenses to avoid photodamage or sun damage (e.g. bleaching) to the product to which they are applied, or to the biological entity to which sunprotection is to be offered. The compounds of the invention may be applied to such materials or impregnated into those materials.

The invention thus further extends to a method of preparing a photoprotective or photoprotected product comprising applying a compound or composition of the invention to said product, or impregnating said product with said compound or composition. The use of compounds or composition of the invention to prepare such products is also considered an object of the invention. Photoprotected or photoprotective products thus formed form further aspects of the invention.

Preferably the compositions of the invention are pharmaceutical compositions comprising a compound as described hereinbefore and one or more pharmaceutically acceptable excipients and/or diluents as described hereinafter.

The compounds described herein have photoprotective, colouring and antioxidant properties.

The compositions as described herein may thus be used in cosmetic or medical applications. The pharmaceutical composition described herein may therefore be a cosmetic composition, an antioxidant composition or a light protection filter or sunscreen. The present invention further provides such compositions for use as a medicament.

The compounds described herein have an attractive golden colour and therefore may be used in cosmetics which take advantage of that colouring or add an additional property to sunscreens of the invention. Thus the sunscreen and/or cosmetic preparations described herein preferably have 2 or more properties, selected from colouring, sunscreen and antioxidant properties. As an alternative or complementary to this property as a colourant the compounds may be used for their antioxidant or photoprotective properties.

Thus in a further aspect the present invention provides compositions as described herein as a cosmetic, sunscreen (light protection filter) or antioxidant.

As referred to herein, a “cosmetic” refers to a composition used on a human or non-human animal for non-medical purposes.

As used herein a “sunscreen” or “light protection filter” or “photoprotective composition” refers to a composition which is suitable for administration to an individual which provides protection against light irradiation (i.e. acts as a light or sun-absorbing compound), particularly of ultraviolet and visible light, preferably wavelength 280-700 nm, especially preferably at least 350-500 nm, e.g. 370-500 nm, 375-490 nm, 400-480 nm, 400-500 nm or 425-475 nm.

Preferably at least one compound in said composition is capable of achieving protection in these wavelength ranges. Protection may be assessed by various techniques, including the time taken to develop a light induced response or the severity of that response, e.g. erythema or burns, e.g. using the currently available tests to determine SPF ratings. When such a test is performed, preferably the composition achieves a SPF of at least 2, preferably at least 10, 20, 30 or 50.

Conveniently however, in order to test efficacy e.g. to filter light of wavelengths that do not significantly result in such responses (e.g. UVA, particularly long-wavelength UVA, ie. 340-400 nm), in vitro tests may be conducted such as filtering of light through filters (to simulate skin) comprising compounds of interest, or determining the extinction coefficient, to determine the ability of those compounds to absorb radiation. In methods which employ a filter comprising the test compound, the efficacy of absorption may be determined directly or indirectly by assessing the level of radiation (e.g. of a particular wavelength) passing through the filter or by assessing the effect of that radiation passing through a filter with or without the test compound, e.g. on cells which are sensitive to radiation and show a response to such radiation.

Preferably in such tests, (e.g. as described in the Examples), said compounds prevent more than 40%, preferably more than 50 or 60% transmission at a given wave-length. Preferred compounds for use in compositions of the invention preferably exhibit maximal absorption in the 375-490 nm range, e.g. >1.5 to 2 times greater absorption at a given wavelength in the 375-490 nm range compared to absorption at 350 nm.

Appropriate techniques for in vitro analysis involve the application of a test compound to a substrate which preferably simulates skin (e.g. a collagen substrate or a quartz plate with simulated skin topography) which is then irradiated with radiation reflecting full solar radiation or preferably narrower wavelength radiation, e.g. using a Xenon arc to simulate the solar UV spectrum, e.g. 290-400 nm.

The UV absorbance of the test compound may be measured, e.g. using a Labsphere UV-1000S UV transmitter analyzer (Labsphere Inc., North Sutton, N.H.). The ability of the test compound to absorb UVA as assessed by e.g. critical wavelength determination (as described by Diffey et al., 2000, J. Am. Acad. Dermatol., 43(6), p 1024-1035) provides an indication of the efficacy of the test compound to absorb in the UV range of the spectrum. Preferably the critical wavelength is more than 360 nm, especially preferably >370 or 380 nm, especially in combination with the SPF values described above.

The invention thus provides a method of treating or preventing the effects of irradiation in (on or of) a human or non-human animal wherein a pharmaceutical compound or composition as described hereinbefore is administered to said animal. Alternatively stated, the present invention provides the use of a pharmaceutical compound or composition as described herein in the preparation of a medicament for treating or preventing the effects of irradiation of a human or non-human animal body. In a further alternative statement, the present invention provides a pharmaceutical compound or composition as described herein for use in treating or preventing the effects of irradiation of a human or non-human animal body.

In a preferred aspect the invention provides a method of treating or preventing the effects of solar radiation on a human wherein a pharmaceutical compound or composition as described hereinbefore is topically administered to the skin or hair of said human. This method serves to protect the skin or hair from the deleterious effects of said solar radiation.

As used herein, “irradiation” refers to direct or indirect irradiation from one or more natural or synthetic light sources, particularly from the sun, i.e. solar radiation. Preferably said radiation is of light in the range 280-700 nm, especially preferably at least 350-500 nm, e.g. 375-490 nm, 400-480 nm, 400-500 nm or 425-475 nm. The “effects” of irradiation may be damaging effects including burns, erythema, premature aging and wrinkling of the skin (dermatoheliosis), development of pre-malignant lesions (solar keratoses) and various malignant tumours or other effects which are undesirable for, for example, cosmetic reasons, e.g. melanin deposition.

As used herein, “treating” refers to the reduction, alleviation or elimination, preferably to normal non-irradiated levels, of one or more of the symptoms or effects of said irradiation e.g. presence or extent of burning or pigmentation, relative to the symptoms or effects present on a different part of the body of said individual not subject to irradiation or in a corresponding individual not subject to irradiation. “Preventing” refers to absolute prevention, or reduction or alleviation of the extent or timing (e.g. delaying) of the onset of that symptom or effect.

The method of treatment or prevention according to the invention may advantageously be combined with administration of one or more active ingredients which are effective in treating or preventing the effects of irradiation. Preferably such additional active ingredients include sunscreen agents (as described herein and as known in the art), antioxidants, vitamins and other ingredients conventionally employed in sunscreen and cosmetic preparations of the art.

Thus, pharmaceutical compositions of the invention may additionally contain one or more of such active ingredients.

According to a yet further aspect of the invention we provide products containing one or more compounds as herein defined and one or more additional active ingredients as a combined preparation for simultaneous, separate or sequential use in human or animal therapy.

The compositions of the invention may be formulated in conventional manner with one or more physiologically acceptable carriers, excipients and/or diluents, according to techniques well known in the art using readily available ingredients. Where appropriate compositions according to the invention are sterilized, e.g. by γ-irradiation, autoclaving or heat sterilization, before or after the addition of a carrier or excipient where that is present, to provide sterile formulations. Thus, the active ingredient may be incorporated, optionally together with other active substances as a combined preparation, with one or more conventional carriers, diluents and/or excipients, to produce conventional galenic preparations such as tablets, pills, powders, lozenges, sachets, cachets, elixirs, suspensions (as injection or infusion fluids), emulsions, solutions, syrups, aerosols (as a solid or in a liquid medium), ointments, soft and hard gelatin capsules, suppositories, sterile injectable solutions, sterile packaged powders, and the like. Biodegradable polymers (such as polyesters, polyanhydrides, polylactic acid, or polyglycolic acid) may also be used for solid implants. The compositions may be stabilized by use of freeze-drying, undercooling or Permazyme.

Suitable excipients, carriers or diluents are lactose, dextrose, sucrose, sorbitol, mannitol, starches, gum acacia, calcium phosphate, calcium carbonate, calcium lactose, corn starch, aglinates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methylhydroxybenzoates, propyl hydroxybenzoates, talc, magnesium stearate, mineral oil or fatty substances such as hard fat or suitable mixtures thereof. Agents for obtaining sustained release formulations, such as carboxypolymethylene, carboxymethyl cellulose, cellulose acetate phthalate, or polyvinylacetate may also be used.

The compositions may additionally include lubricating agents, wetting agents, emulsifying agents, viscosity increasing agents, granulating agents, disintegrating agents, binding agents, osmotic active agents, suspending agents, preserving agents, sweetening agents, flavouring agents, adsorption enhancers (e.g. surface penetrating agents or for nasal delivery, e.g. bile salts, lecithins, surfactants, fatty acids, chelators), browning agents, organic solvent, antioxidant, stabilizing agents, emollients, silicone, alpha-hydroxy acid, demulcent, anti-foaming agent, moisturizing agent, vitamin, fragrance, ionic or non-ionic thickeners, surfactants, filler, ionic or non-ionic thickener, sequestrant, polymer, propellant, alkalinizing or acidifying agent, opacifier, colouring agents and fatty compounds and the like.

The compositions of the invention may be formulated so as to provide quick, sustained or delayed release of the active ingredient after administration to the body by employing techniques well known in the art.

The composition may be in any appropriate dosage form to allow delivery or for targetting particular cells or tissues, e.g. as an emulsion or in liposomes, niosomes, microspheres, nanoparticles or the like with which the active ingredient may be absorbed, adsorbed, incorporated or bound. This can effectively convert the product to an insoluble form. These particulate forms may overcome both stability (e.g. degradation) and delivery problems.

These particles may carry appropriate surface molecules to improve circulation time (e.g. serum components, surfactants, polyoxamine908, PEG etc.) or moieties for site-specific targeting, such as ligands to particular cell borne receptors. Appropriate techniques for drug delivery and for targeting are well known in the art and are described in WO99/62315.

The use of solutions, suspensions, gels and emulsions are preferred, e.g. the active ingredient may be carried in water, a gas, a water-based liquid, an oil, a gel, an emulsion, an oil-in water or water-in-oil emulsion, a dispersion or a mixture thereof.

Compositions may be for topical (e.g. to the skin or hair), oral or parenteral administration, e.g. by injection. Topical compositions and administration are however preferred, and include gels, creams, ointments, sprays, lotions, salves, sticks, soaps, powders, films, aerosols, drops, foams, solutions, emulsions, suspensions, dispersions e.g. non-ionic vesicle dispersions, milks and any other conventional pharmaceutical forms in the art.

Ointments, gels and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or colouring agents. Powders may be formed with the aid of any suitable powder base. Drops and solutions may be formulated with an aqueous or non-aqueous base also comprising one or more dispersing, solubilising or suspending agents. Aerosol sprays are conveniently delivered from pressurised packs, with the use of a suitable propellant.

Alternatively, the compositions may be provided in a form adapted for oral or parenteral administration. Alternative pharmaceutical forms thus include plain or coated tablets, capsules, suspensions and solutions containing the active component optionally together with one or more inert conventional carriers and/or diluents, e.g. with corn starch, lactose, sucrose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fatty substances such as hard fat or suitable mixtures thereof. The concentration of active ingredient in compositions of the invention, depends upon the nature of the compound used, the mode of administration, the course of treatment, the age and weight of the patient, the cosmetic or medical indication, the body or body area to be treated and may be varied or adjusted according to choice. Generally however, concentration ranges for the compound described herein is 0.0005, 0.001 or 0.01 to 25%, e.g. 0.05 to 1% or 0.01 to 10%, such as 0.1 to 5, e.g. 1-5% (w/w of the final preparation for administration, particularly for topical administration). Said concentrations are determined by reference to the amount of the compound itself and thus appropriate allowances should be made to take into account the purity of the composition. Effective single doses may lie in the range of from 1-100 mg/day, preferably 2-10 mg/day, depending on the animal being treated, taken as a single dose.

The administration may be by any suitable method known in the medicinal arts, including for example oral, parenteral (e.g. intramuscular, subcutaneous, intraperitoneal or intravenous) percutaneous, buccal, rectal or topical administration or administration by inhalation. The preferred administration forms will be administered orally, or most preferably topically. As will be appreciated oral administration has its limitations if the active ingredient is digestible. To overcome such problems, ingredients may be stabilized as mentioned previously.

Administration may be conducted before, during or after irradiation to offer prevention or treatment of the effects of irradiation. Thus for example the composition may be administered orally or applied topically up to e.g. 1 day, but preferably less than 1 hour before irradiation, at any time during irradiation and post-irradiation, e.g. in the 12 hours post-irradiation.

Sunscreen formulations may be presented as topical formulations as described hereinbefore, particularly as body, face or lip milks, foams, sprays, lotions, gels or balms. Depending on their formulation and the compound used in the composition, sunscreen preparations of the invention may also have cosmetic properties, e.g. by the inclusion of additional components or the selection of a coloured compound of the invention. Similarly, cosmetic preparations as described herein may have sunscreen properties.

The present invention also extends to particular cosmetic compositions or preparations (personal care products) comprising the compositions described hereinbefore. Such preparations may take the form of make-up products (such as eye or face products, including eye shadow, powder, lipstick, foundation, mascara, blush, eyeliner, nail polish, tinted creams and foundations, sun make-up), creams, lotions or colourants. Preferably such preparations are in the form of an anhydrous or aqueous solid or paste. The carotenoids of the invention may be used to impart colour, sunscreen and/or antioxidant properties to such preparations. For sunscreen products, the compositions may be as described hereinbefore particularly for topical administration to the skin. For the treatment or protection of hair, the composition may be in the form of a hair rinse, spray mist, gel, mousse, shampoo, conditioner, lotion, emulsion or colouring product.

The invention thus further extends to a method of preparing the above described sunscreen or cosmetic preparation comprising adding a compound or composition as described hereinbefore to a pharmaceutically acceptable diluent, carrier and/or excipient or base sunscreen or cosmetic, wherein the base sunscreen or cosmetic may comprise ingredients which impart photoprotective and/or cosmetic, e.g. colouring, properties. The use of compounds or composition of the invention to prepare such cosmetics/sunscreens is also considered an object of the invention.

Animals to which the compositions may be applied or administered include mammals, reptiles, birds, insects and fish which suffer deleterious effects from light irradiation. Preferably the animals to which the compositions of the invention are applied are mammals, particularly primates, domestic animals, livestock and laboratory animals. Thus preferred animals include mice, rats, rabbits, guinea pigs, cats, dogs, monkeys, pigs, cows, goats, sheep and horses. Especially preferably the compositions are applied or administered to humans.

“Body coverings” or “body surfaces” to which the compositions of the invention may be applied include body coverings such as skin, bodily outgrowths such as hair and nails and surfaces such as mucosal membranes, but also include equivalents in other animals such as scales or feathers.

The following Examples are given by way of illustration only in which the Figures referred to are as follows:

FIG. 1 shows the chemical structure of (A) sarcinaxanthin (I), sarcinaxanthin monoglucoside (II) and sarcinaxanthin diglucoside (III), (B) 7,8-dihydrosarcinaxanthin and (C) sarcinaxanthin succinate;

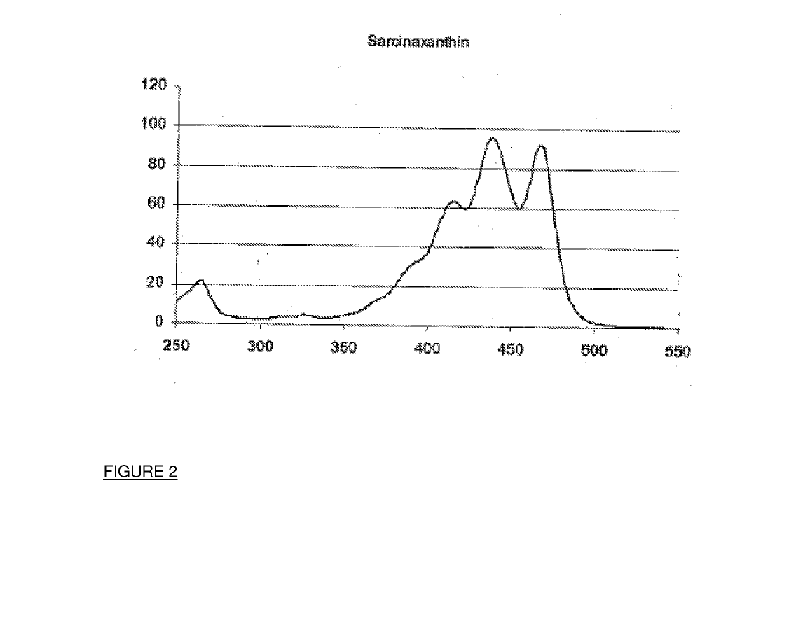

FIG. 2 shows the absorption spectrum of sarcinaxanthin;

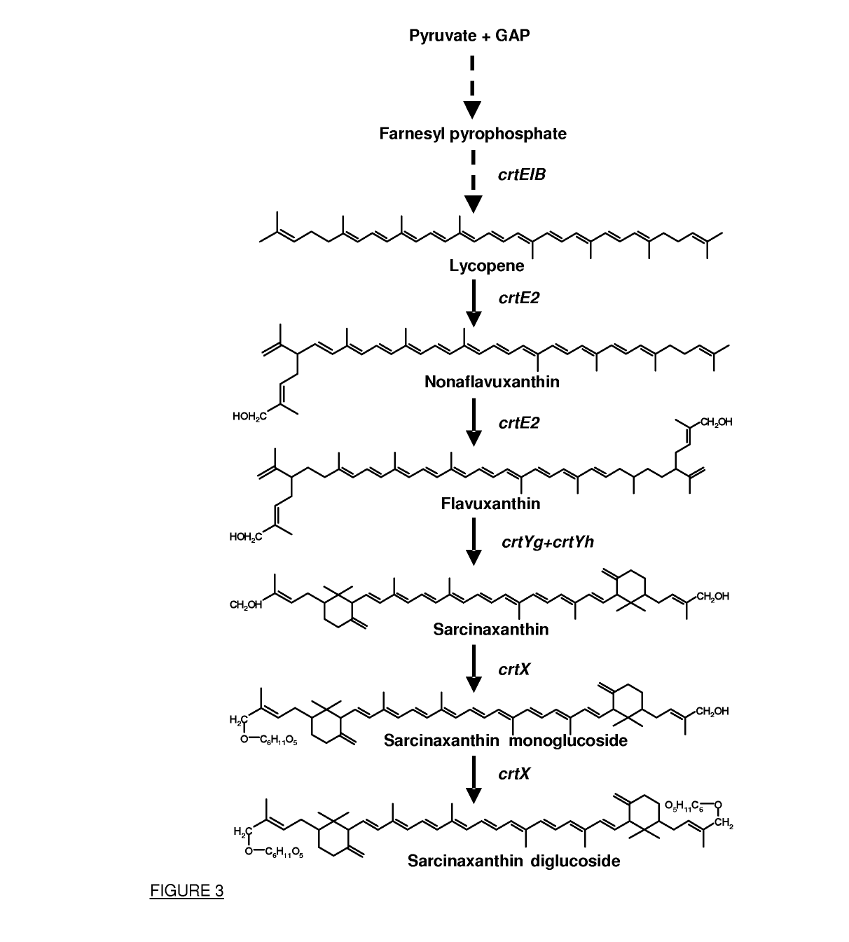

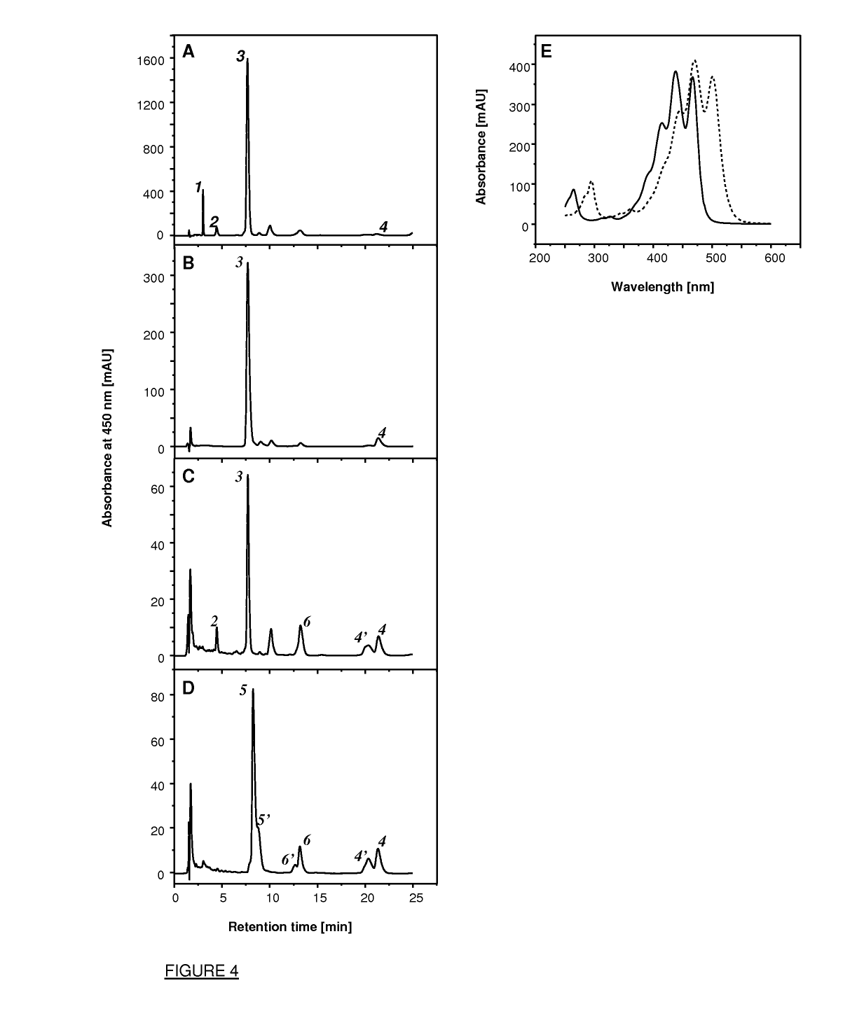

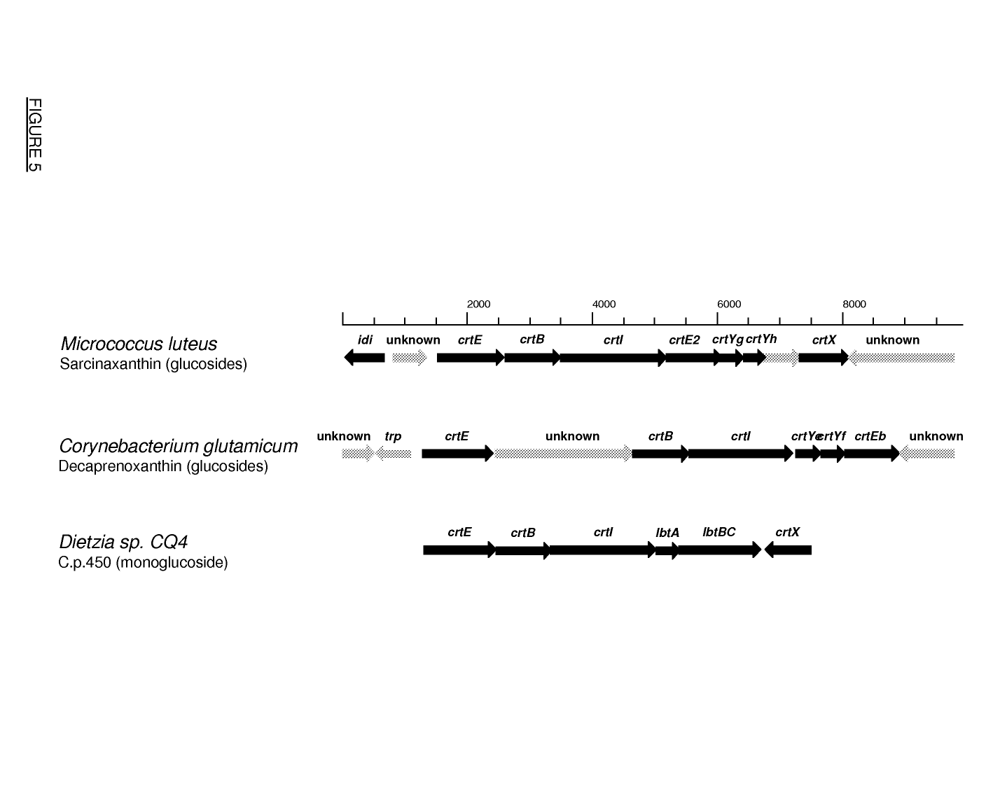

FIG. 3 shows the proposed biosynthetic pathway for the individual steps in the formation of sarcinaxanthin and its glucosides from lycopene. crtEBI: GGPP synthase, phytoene synthase, phytoene desaturase; CrtE2: lycopene elongase; CrtYg+CrtYf: C₅₀ carotenoid γ-cyclase; CrtX: C₅₀ carotenoid glycosyl transferase;

FIG. 4 shows the HPLC elution profile of carotenoids extracted from M. luteus strain Otnes7 (A), lycopene-producing E. coli XL1 Blue pAC-LYC transformed with pCRT-E2YgYh-O7 (B), pCRT-E2YgYhX-O7 (C) and pCRT-E2-O7 (D). Peak 1, sarcinaxanthin diglucoside; peak 2, sarcinaxanthin monoglucoside; peak 3, sarcinaxanthin; peak 4, lycopene; peak 5, flavuxanthin; peak 6, nonaflavuxanthin; Peak 4′ 5′ and 6′ are the cis isomers of 4, 5 and 6 respectively. Absorption spectra of carotenoids from peaks 1, 2 and 3 (solid line) and peaks 4, 5 and 6 (scattered line) are depicted in graph (E);

FIG. 5 shows the carotenoid biosynthesis gene clusters from M. luteus, C. glutamicum and Dietzia sp. leading to C₅₀ carotenoids sarcinaxanthin, decaprenoxanthin, C.p.450 and its glycosylated derivatives, respectively. Genes indicated in grey are suggested not to be involved in carotenoid biosynthesis;

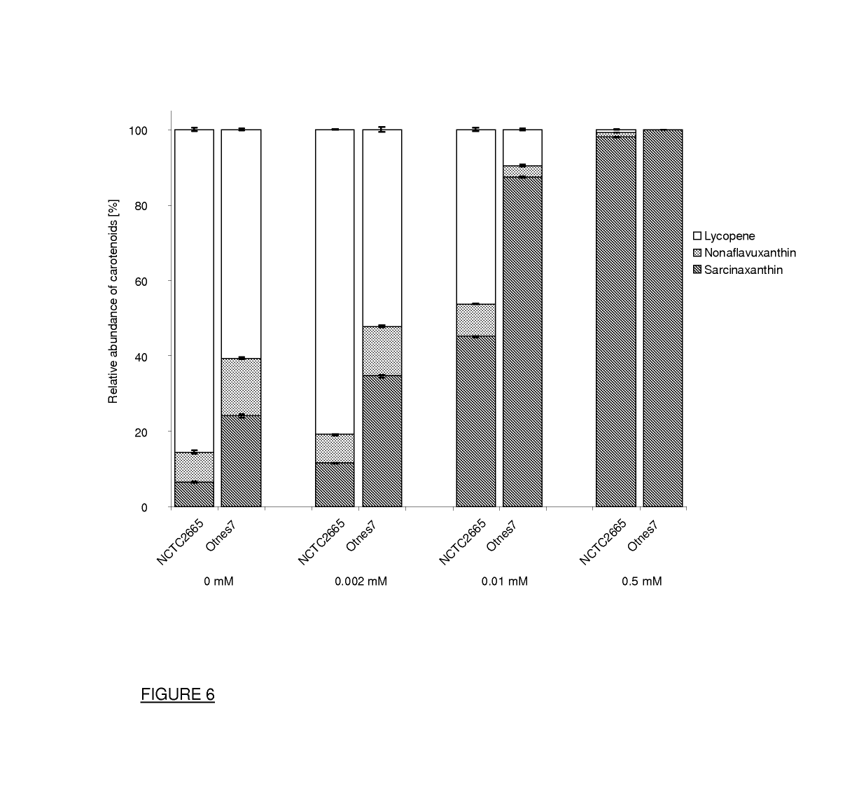

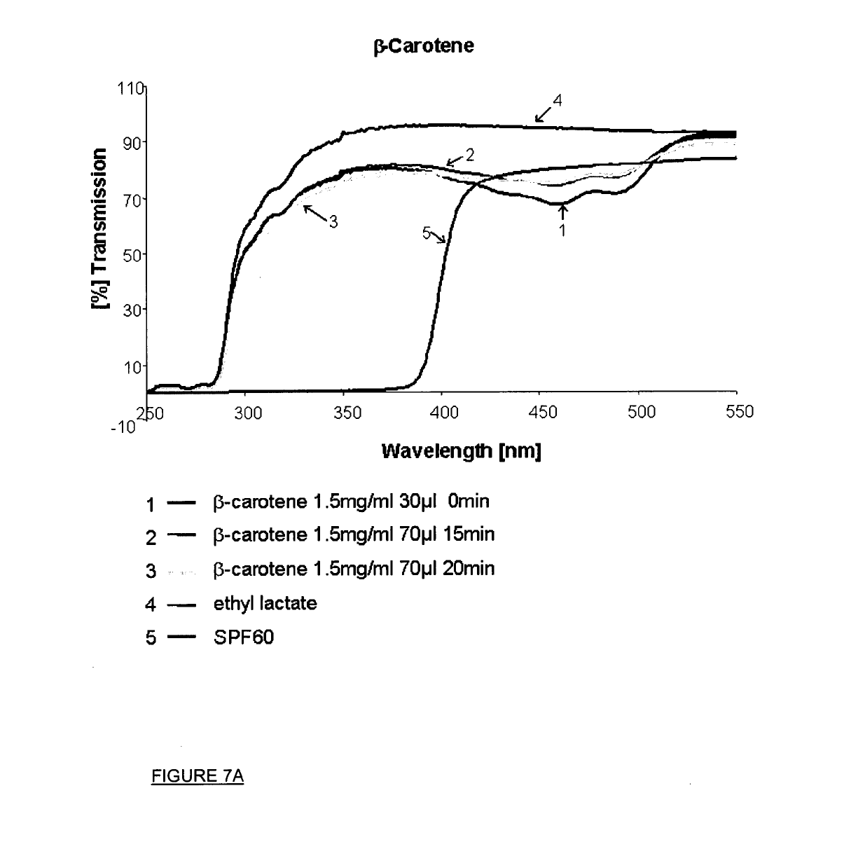

FIG. 6 shows the relative carotenoid abundance in extracts from E. coli pAC-LYC overexpressing crtE2YgYh genes from M. luteus strain Otnes7 and strain NCTC2665 cultivated in the presence of 0, 0.002, 0.01 and 0.5 mM m-toluate. The fraction of sarcinaxanthin, lycopene and intermediates are indicated by dark grey, white and light grey columns, respectively. Samples were analyzed after 48 h of cultivation. The extracted total carotenoid was similar in the presented samples and 100% carotenoid abundance corresponds to [x]±[y] mg/g cell dry weight (CDW) total carotenoid;

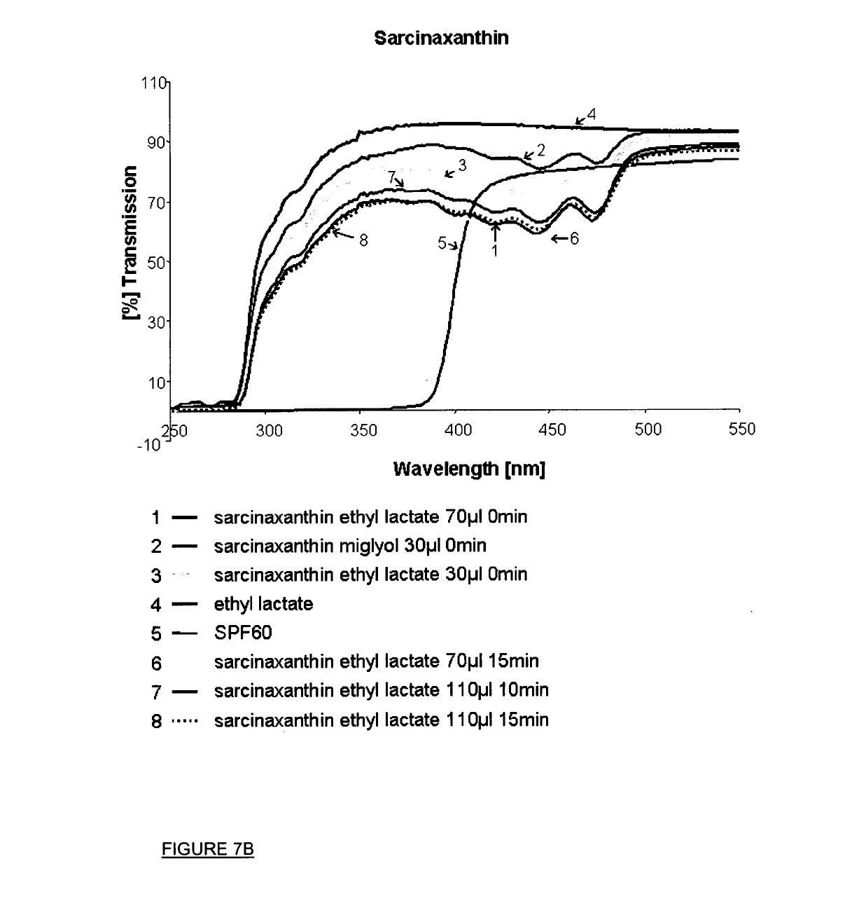

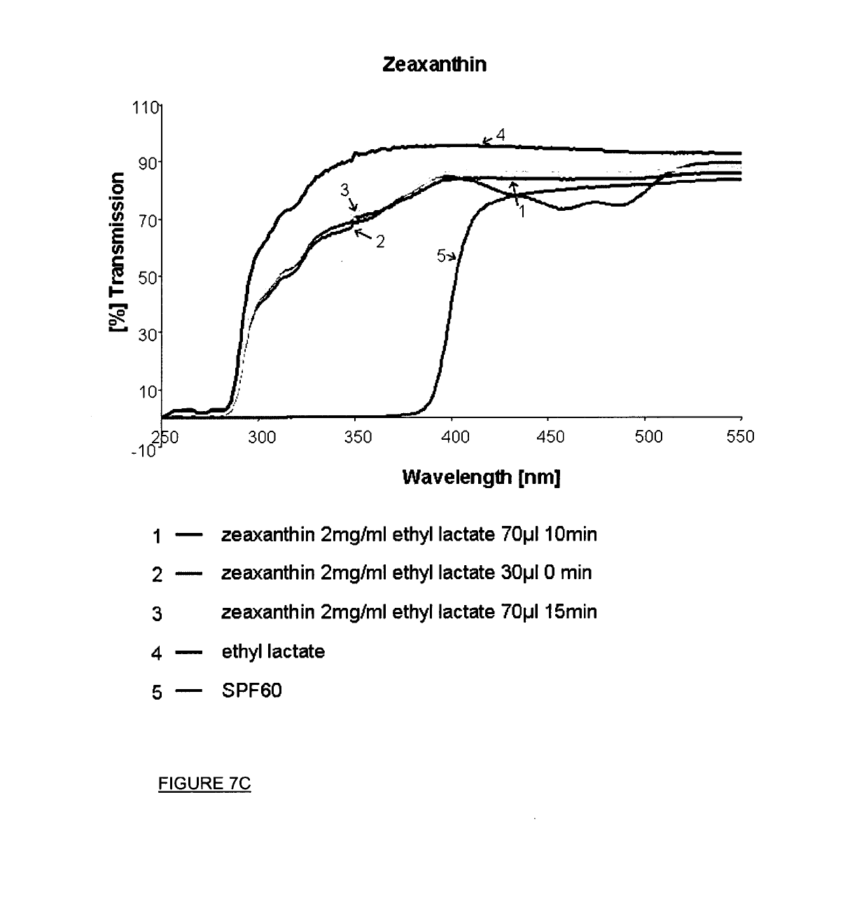

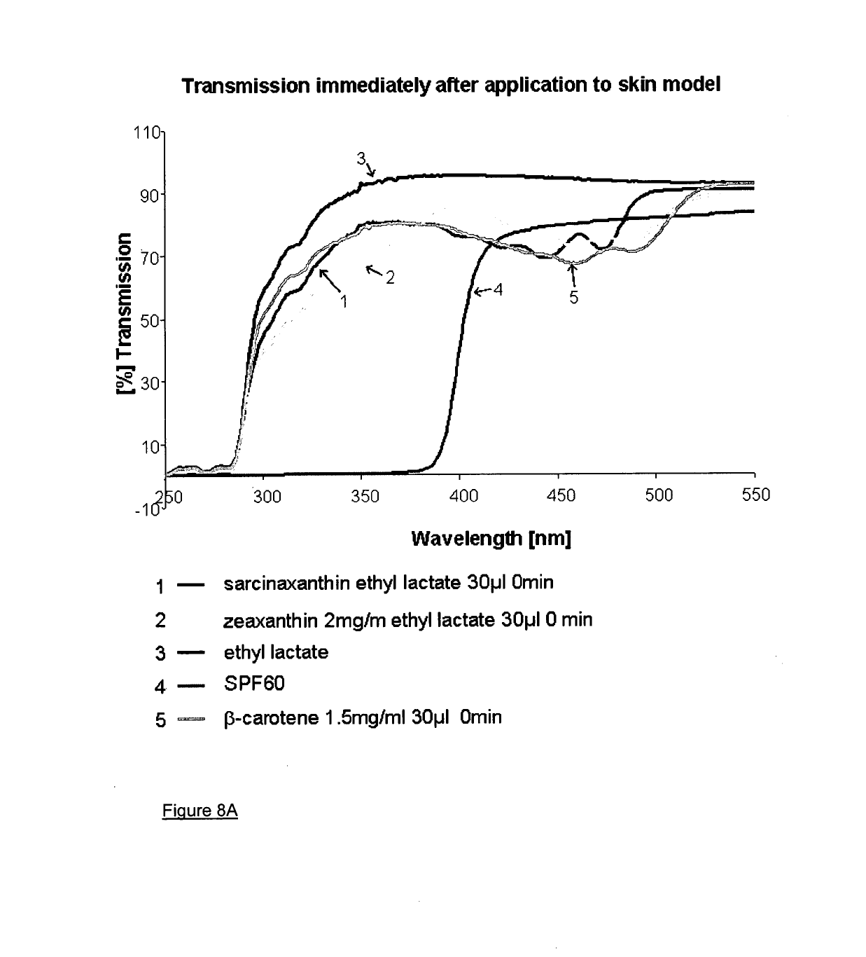

FIG. 7 shows the transmission spectra from Integrating sphere analysis using (A) β-carotene, (B) sarcinaxanthin and (C) zeaxathin at the concentrations and for the times indicated. A commercial sunscreen SPF60 was used for comparison as well as the diluent as indicated in the key. Vitro-skin+ ethyl lactate was used as the control; and

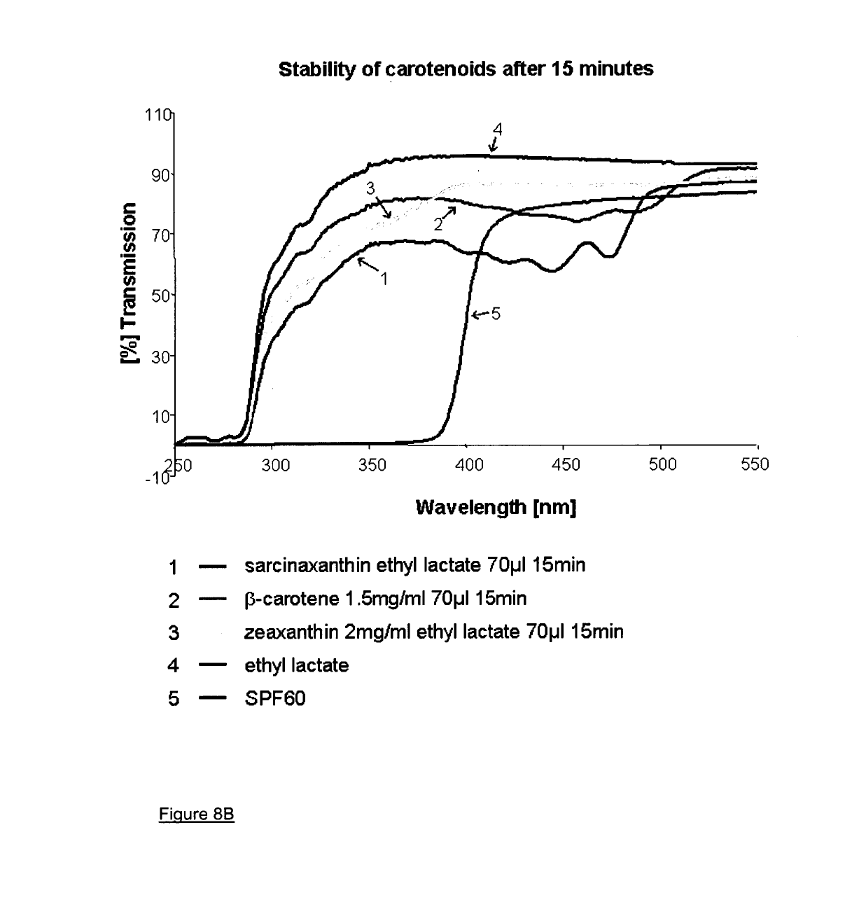

FIG. 8 shows the transmission spectra for Integrating sphere analysis for sarcinaxanthin, β-carotene and zeaxanthin (with controls as for FIG. 7 and with the diluent as shown in the key) in which in (A) % transmission was measured immediately on application of the test compounds to the skin model and (B) after 15 minutes.

### SEQUENCES

| SEQ ID NO: 1 | |
|---|---|
| M. luteus NCTC2665 sarcinaxanthin gene cluster | |
| 1 | gcggagtcct cgtccgcctc ggcgtcgtcg ctgtccgcgg ccccggccga ctacgaggcc |
| 61 | ggcacgtgct tcaccgcccc gctcggcgcg cgtgacctgt cctccttcga gaccaccgac |
| 121 | tgcgagggcg cccacaccgc ggagtacctg tgggccgtgc cggccgtggc cgagggtgag |
| 181 | gaggccgacc ccgccgccgc ccagacctgc accgcccagg cccagcgcct gagcgaggag |
| 241 | aaggaggacc agctgaacgg ggccgtcctg acctcctccg agctgggcaa ctacggcacc |
| 301 | gacgagaagc actgcgtcgt gtacggggtc tccggtgagt gggagggtca gatcgtggac |
| 361 | ccggagatca ccctggagac ggcgtccgcc gacgcctgat cccgccggcg gccccgtgcg |
| 421 | tcgtgagatc gcgccgcccg ggaccgccgc ggatggacgc gggaccggcg cggcccgtag |
| 481 | tgtcttctgc gtccagaagt tagacggtcg aacaggtgcg gcggtcggtg ccgcgtcgtg |
| 541 | tccgccaccg aggaggcgcc atgggtgaag cgaggacggg cggcgaggcc gcgctctccg |
| 601 | gggtgaccgc cgagctggac gccgcgctcc gacacgccgc ggcccaggcg cccggatccg |
| 661 | ccgccttcgc cgagctgctc gactcgctcc acgtccatgt gggcgccggc aagctcatcc |
| 721 | gcccccgtct cgtcgagctc ggctggcgcc tggcgaccgc cgacccggtc cctccgtccg |
| 781 | gccgcgctgc cgtcgaccga ctcggggccg ccttcgaact gctgcacacc gcgctgctcg |
| 841 | tccacgacga cgtcatcgat cgggacgtgc tgcggcgcgg ccagcccgcc gtgcacgcct |
| 901 | ccgcccggca ccgcctcgag gcccgcgggg tgcccgccgc ggacgccgcc cacgccgggg |
| 961 | tcgccgtcgc cctcatcgcg ggggacgtcc tgctcaccca ggcgttccgg ctcgccgcca |
| 1021 | cctgtgccgc cgacaccgcc cgggccgccg aggccgccgc cgtcgtcttc gacgccgccg |
| 1081 | ccgtgactgc ggccggcgag ctcgaggacg tgctcctggg gctgtcccgc cacaccggtg |
| 1141 | aggagcccga tcccgaccgc atcctcgcca tgcaacggct caagacggcg cactacacgg |
| 1201 | tcggcgcgcc cctgcgcgcc ggcgccctcc tggccggggc ggatcccgac ctcgcccggg |
| 1261 | cgatgggcga ggccggcgcc gacctcggcg ccgcctacca ggtgatcgac gacgtcctcg |
| 1321 | gcgtgttcgg cgatcccggg gagaccggca agtccgccga cggcgacctg cgcgagggca |
| 1381 | aggccaccgt gctcaccgcc cacggccgcc gcatccccgc cgtccgcgcc ctgctcgacg |
| 1441 | cgggcccggc cacccccgcg gacatcgagg ccgcccgccg cgccctcgag gcggccggtg |
| 1501 | cccgggagca cgccctcgac gtcgccgccg agctcaccgt ccgcgcccgc gagcgcatcg |
| 1561 | cggccctgcc cctggacgag acggtccggg cggagttcgc cgacgcctgc cacgccgtgc |
| 1621 | tgacccggag gtcctgagat ggccgcgccc accccgagcc ctgccgcgct gtacacgcgg |
| 1681 | acggcccaca ccgcagcggc ccaggtgatc cgccgctact ccacgtcctt ctcctgggcc |
| 1741 | tgccgcaccc tgccccggca ggcacgccag gacgtggcca cgatctacgc catggtccgc |
| 1801 | gtcgccgacg aggtggtcga cggcgtcgcg gtggccgccg ggctcgacga ggccggggtc |
| 1861 | cgcgccgccc tggacgacta cgagcgggcg tgtgaggccg cgatggcgtc gggcttcgcc |
| 1921 | accgacccgg tcctgcacgc cttcgccgac gtggcccgtc gccacggcat caccccggag |
| 1981 | ctgacccgtc ccttcttcgc ctccatgcgc gcggacctgg ggatccgcga gcacggcgcc |
| 2041 | gagtccctgg acgcctacat ccacggctcg gccgaggtgg tggggctgat gtgcctgcag |
| 2101 | gtcttcctct ccctccccgg cacgcgggcc cggaccccgg gccagcggca ggagctgcgc |
| 2161 | gcgcaggcct cccggctggg ggcggcgttc cagaaggtca acttcctcag ggacctggcc |
| 2221 | gcggaccacc acgagctggg ccgcacctac ctgcccggtg ccgcaccggg cgtgctcacc |
| 2281 | gaggcccgca aggccgagct cgtggccgag gtccgcgccg acctcgacgc cgccctgccc |
| 2341 | ggcatccgtg tcctggaccc cggggccggg cgcgccgtgg ccctggcgca cggactgttc |
| 2401 | gcggccctgg tggaccggat cgaggcgacc ccggcggccg agctggccca ccgccgtgtc |
| 2461 | cgggtgccgg accatcagaa ggcccggatc gccgcccgcg tcctggcacg gggccgccgg |
| 2521 | ggaggccgcc gatgagcgcc cgggacaccg ctctcggccc gcgcaccgtg gtggtgggcg |
| 2581 | gcggtttcgc cggactggcc acggcgggcc tgttggcccg cgacgggcac cgggtgacgc |
| 2641 | tgctggagcg cggcgccgtc ctgggcggcc gtgccggacg ctggtccgag gcggggttca |
| 2701 | ccttcgatac cgggccctcc tggtacctga tgcccgaggt gatcgaccgc tggttccgcc |
| 2761 | tcatggggac ctccgccgcc gaacggctgg acctgcgccg tctggacccc ggctaccggg |
| 2821 | tgtacttcga ggggcacctc cacgagcccc ccgtggacgt gcgcaccggc cacgcggaga |
| 2881 | cgctgttcga gtccctcgag cccggcgccg ggcgccggct gcgggcctac ctcgactccg |
| 2941 | cgtcccggat ctacgggctc gccaaggagc acttcctcta cacggacttc cgccggccgg |
| 3001 | ccgccctggc ccacccggac gtcctgcgcg ccctgccggc cctcgggccc cagctgctgg |
| 3061 | ggggcctgcg ctcccacgtc gcggcccgct tccaggaccc ccggctgcgc cagatcctgg |
| 3121 | gctacccggc ggtcttcctc ggcacgtccc ccgaccgtgc ccccgccatg taccacctga |
| 3181 | tgtcccatct ggacctcgcc gacggcgtgc agtaccccct cggcgggttc gcggccctcg |
| 3241 | tggacgccat ggcggaggtc gtgcgcgagg ccggcgtgga gatccgcacc ggggtcgagg |
| 3301 | cgaccgccgt ggaggtcgcg gaccgtcccg cccccgccgg ccgcctcgga cgcctggccg |
| 3361 | cccgcctgcc caggccggga gcagcccgcg gggacgaggg ccgacgtcgc cgcccgggcc |
| 3421 | gggtgaccgg cgtcgcctgg cggtccgacg acggcgccgc gggacgcctc gacgccgatg |
| 3481 | tggtggtggc cgccgcggac ctgcaccacg tgcagacccg tctgctgcct cccggccggc |
| 3541 | gcgtcgcgga gtccacgtgg gaccggcgcg accccggccc ctccggcgtg ctcgtgtgcg |
| 3601 | tgggggtgcg cggatccctg ccccagctgg cccatcacac cctgctgttc acggcggact |
| 3661 | gggaggacaa cttcgggcgc atcgagcggg gggaggacct cgccgcggac acgtcgatct |
| 3721 | acgtctcgcg cacctccgcc acggacccgg gcgtggcccc ggagggcgac gagaacctct |
| 3781 | tcatcctcgt cccggccccc gccgagccgg ggtgggggcg cggcggcatc cgggtccgtg |
| 3841 | acggccaggg ctggcgggtg gaccgcgccg gggacgccca ggtggaggcc gtggcggacc |
| 3901 | gggccctcga tcagctggcc cgctgggccg ggatccccga cctggccgag cgcatcgtgg |
| 3961 | tgcggcgcac ctacgggccc ggtgacttcg ccgcggacgt gcacgcctgg cggggttcgc |
| 4021 | tgctgggccc cgggcacacg ctggcgcagt cggccatgtt ccgcccctcg gtgcgggacg |
| 4081 | cggacgtggc cggcctgatg tacgcgggct cctcggtgcg cccgggaatc ggggtgccca |
| 4141 | tgtgcctgat ctccgccgaa gtggtccggg acgaactgcg ccacgacgcg cgcagggccc |
| 4201 | ggcccgcggg ccccgggggg agcggcacat gatccgcacc ctcttctggg tgtcccggcc |
| 4261 | ggtcagctgg gtgaacacgg cctacccgtt cgccgccgcc gcgatcctga ccggggggct |
| 4321 | gcccgcgtgg ctggtggtcc tgggcgtcgt gttcttcctg gtgccctaca acctggccat |
| 4381 | gtacggcatc aatgacgtgt tcgacttcgc ctcggacctg cgcaaccccc gcaagggggg |
| 4441 | tgtggagggc tccgtgctgg gcgaccccgc ggtgcgccgc cgggtgctgg cgtggtcggt |
| 4501 | gctgctgccc gtgccgttcg tggccgtgct cgcgggctgg tccgccgtgc ggggcgagtg |
| 4561 | ggccgccgtg ctggtgctcg cggtgagcct gttcgcggtg gtggcgtact cctgggcggg |
| 4621 | gctgcggttc aaggagcggc ccttcctgga cgccgccacc tccgccaccc acttcgtctc |
| 4681 | ccccgcggtc tacggcctcg cgctggccgg ggcgaccccc acgcccgccc tggcggcgct |
| 4741 | gctgggggcg ttcttcctgt ggggcatggc ctcgcagatg ttcggggcgg tgcaggacgt |
| 4801 | ggtgccggac cgggaggggg gcctggcctc ggtggccacc gtgctgggcg ctcggcgcac |
| 4861 | cgtcctgctc gccgccggcc tgtacgcggc ggcgggcctg ctgctgctgg ccaccgaccc |
| 4921 | gccgggcccg ctcgcggcgc tgctggccgt gccctacgtg gtgaacaccc tgcgcttccg |
| 4981 | ccgcatcacg gacgccacct cgggcgcggc ccaccgcggc tggcagctgt tccttccgct |
| 5041 | gaactacgtg accggcttcc tcgtgaccct gctgctgatc gggtgggcgc tgacccgggg |
| 5101 | ggcggcggca tgatctacct gctggccctg ctgggtgtca tcggctgcat gctgctggtg |
| 5161 | gaccggcgct tcgagctgtt cctgtggcat cgcccgctcc cggcgctgct ggtgctggcc |
| 5221 | gccggggtgg cctacttctt cgcctgggac ctgtggggga tcgccgaagg cgtgttcctg |
| 5281 | caccggcagt cgccctacat gaccggggtg atgctcgccc cccagctgcc cctggaggag |
| 5341 | gggttcttcc tgctcttcct cagccagatc acgatggtgc tgttcaccgg ggcgctgcgc |
| 5401 | ctgctgcgcg gccggcgagg tgacgcccgt gccgcgacgg cggccgatcc gaccgaccgg |
| 5461 | gggagccggt gaccttcctc gacctcgtcc tcgtcttcgt gggcttcgcc ctggccgtgc |
| 5521 | tcgtgggcgc cgccctcgtc ggccgcgtgc ggggcgagca cctgcgggcc gtggcggcca |
| 5581 | ccctggtggc cctgtgggcc ctcacggcgg tcttcgacaa cgtgatgatc gccgcggggc |
| 5641 | tcttcgacta cggccatgag ctgctggtgg gtgcctacgt gggccaggcg cccgtggagg |
| 5701 | acttcgccta cccgctcggc tccgccctgc tgctgccggc gctctggctg ctgctgacga |
| 5761 | gccgtcgtgc cgatcggcgc ggccgtcggc cgggacgccg cccccacccg gacgatcgct |
| 5821 | gacatgctgc cgttgatccc cgcagacctg ctgcgcgcgc tcggcctgat cctcgtcccg |
| 5881 | gtcgcggcgg tgcacgccgg atggccgtcc gcggcggcga tgctgctcgt gttcggctcc |
| 5941 | cagtggctca cccgctggct cgccccgggc ggcgccctgg actgggccgc gcaggcggtc |
| 6001 | ctgctgctgg ccgggtggct gagcgtcatc ggcctctacc cgcgggtgcc gtggctggac |
| 6061 | ctgctcgtgc acgccgccgc ctccgccgtg gtcgcctgtc tgacggcact ggtggtgggg |
| 6121 | gcgtggctcc ggcgtcgggg gaccgaggcc gggcaggccg tggcgctgct cggcccgggc |
| 6181 | ctggccgggc tggggatcgc ggccgccgcc gtggccctgg gcgtggtgtg ggagctggcc |
| 6241 | gaatggtggg ggcacacggc ggtgaccccg gagatcggcg tgggctacac ggacaccatc |
| 6301 | ggcgacctcg ccgccgatct cgtcggcgcc ggggtcggcg ccgccctcgc cgtgtgccgg |
| 6361 | gggcgcaccc ggtgaccccg gcccgcccca cggtctccgt ggtcgtcccg gtgctcgacg |
| 6421 | acgccgagca cctgcgcgtg tgcctcgcgc tgctggccgc ccagagccgg ccggcgctgg |
| 6481 | aggtggtggt ggtggacaac ggctgcgtgg acgactcggc ggtgctcgcc cgcgccgccg |
| 6541 | gcgcgcgggt ggtgcgcgag ccgcgccgcg gggtcccggc cgcggcggcc gccggcctgg |
| 6601 | acgccgcggt cggggagctg ctggtgcgct gcgacgccga cacgcggatg cccgcggact |
| 6661 | ggctcgaacg gatcgtggcc cggttcgacg ccgaccccgg gctcgacgcc ctcaccgggc |
| 6721 | cggggacctt ccacgaccag cccggcctcc ggggacaggt gcgggcggcg ctctacaccg |
| 6781 | gcacgtaccg ctggggggcg ggcgccgcgg tggcggccac ccccgtctgg ggctccaact |
| 6841 | gcgccctgcg cgccgaggcg tggcaggctg tgcggacccg cgtccaccgc gaacgcgggg |
| 6901 | acgtgcacga tgacctggac ctgtccttcc agctggccct ggccggccgc cggatccggt |
| 6961 | tcgatccgga cctgcgggtg gaggtcgccg ggcgcatctt ccactccctg cgccagcggg |
| 7021 | tgcggcaggg ccggatggcg gtcaccaccc tgcaggtcaa ctgggcccga ctgtcccccg |
| 7081 | ggcggcgttg gctgcgccgg gcggcccggg cacacccccg gtcccgctgg gggcgtggcc |
| 7141 | ccgacggtca gtcccgggac tga |
| SEQ ID NO: 2 | |
| M. luteus NCTC2665 crtYg nucleotide sequence | |
| atgatctacctgctggccctgctgggtgtcatcggctgcatgctgctggtggaccggcgcttcgagctgttcctgtggca | |
| tcgcccgctcccggcgctgctggtgctggccgccggggtggcctacttcttcgcctgggacctgtgggggatcgccg | |
| aaggcgtgttcctgcaccggcagtcgccctacatgaccggggtgatgctcgccccccagctgcccctggaggagg | |
| ggttcttcctgctcttcctcagccagatcacgatggtgctgttcaccggggcgctgcgcctgctgcgcggccggcgag | |
| gtgacgcccgtgccgcgacggcggccgatccgaccgaccgggggagccggtga | |
| SEQ ID NO: 3 | |
| M. luteus NCTC2665 crtYg polypeptide sequence | |
| MIYLLALLGVIGCMLLVDRRFELFLWHRPLPALLVLAAGVAYFFAWDLWGIAEGVF | |
| LHRQSPYMTGVMLAPQLPLEEGFFLLFLSQITMVLFTGALRLLRGRRGDARAATA | |
| ADPTDRGSR | |
| SEQ ID NO: 4 | |
| M. luteus NCTC2665 crtYh nucleotide sequence | |
| gtgaccttcctcgacctcgtcctcgtcttcgtgggcttcgccctggccgtgctcgtgggcgccgccctcgtcggccgcgt | |
| gcggggcgagcacctgcgggccgtggcggccaccctggtggccctgtgggccctcacggcggtcttcgacaacg | |
| tgatgatcgccgcggggctcttcgactacggccatgagctgctggtgggtgcctacgtgggccaggcgcccgtgga | |
| ggacttcgcctacccgctcggctccgccctgctgctgccggcgctctggctgctgctgacgagccgtcgtgccgatc | |
| ggcgcggccgtcggccgggacgccgcccccacccggacgatcgctga | |
| SEQ ID NO: 5 | |
| M. luteus NCTC2665 crtYh polypeptide sequence | |
| VTFLDLVLVFVGFALAVLVGAALVGRVRGEHLRAVAATLVALWALTAVFDNVMIAA | |
| GLFDYGHELLVGAYVGQAPVEDFAYPLGSALLLPALWLLLTSRRADRRGRRPGR | |
| RPHPDDR | |
| SEQ ID NO: 6 | |
| M. luteus NCTC2665 crtE2 nucleotide sequence | |
| atgatccgcaccctcttctgggtgtcccggccggtcagctgggtgaacacggcctacccgttcgccgccgccgcgat | |
| cctgaccggggggctgcccgcgtggctggtggtcctgggcgtcgtgttcttcctggtgccctacaacctggccatgta | |
| cggcatcaatgacgtgttcgacttcgcctcggacctgcgcaacccccgcaaggggggtgtggagggctccgtgctg | |
| ggcgaccccgcggtgcgccgccgggtgctggcgtggtcggtgctgctgcccgtgccgttcgtggccgtgctcgcgg | |
| gctggtccgccgtgcggggcgagtgggccgccgtgctggtgctcgcggtgagcctgttcgcggtggtggcgtactcc | |
| tgggcggggctgcggttcaaggagcggcccttcctggacgccgccacctccgccacccacttcgtctcccccgcgg | |
| tctacggcctcgcgctggccggggcgacccccacgcccgccctggcggcgctgctgggggcgttcttcctgtgggg | |
| catggcctcgcagatgttcggggcggtgcaggacgtggtgccggaccgggaggggggcctggcctcggtggcca | |
| ccgtgctgggcgctcggcgcaccgtcctgctcgccgccggcctgtacgcggcggcgggcctgctgctgctggcca | |
| ccgacccgccgggcccgctcgcggcgctgctggccgtgccctacgtggtgaacaccctgcgcttccgccgcatca | |
| cggacgccacctcgggcgcggcccaccgcggctggcagctgttccttccgctgaactacgtgaccggcttcctcgt | |
| gaccctgctgctgatcgggtgggcgctgacccggggggcggcggcatga | |
| SEQ ID NO: 7 | |
| C. glutamicum crtEb nucleotide sequence | |
| atgatggaaaaaataagactgattctattgtcatctcgccccattagctgggtcaataccgcctacccttttgggctggc | |
| atacctattaaatgcaggagagattgactggctgttttggctaggcatcgtgttttttcttatcccgtataacatcgccatgt | |
| atggcatcaacgatgtttttgattacgaatctgacatacgtaatccccgcaaaggcggcgtcgagggggccgtgctc | |
| ccgaaaagttcccacagcacactgttatgggcatcggctatctcaacaattcctttcctagttattcttttcatatttggcac | |
| ctggatgtcgtctttatggctgacaatctcagtgctagcagtgattgcttattcagcaccgaaattgcgttttaaagaacg | |
| cccctttatcgatgctctaacatcttctactcacttcacttcacctgcattaatcggtgcaacgatcactggaacatctcctt | |
| cagcagcgatgtggatagcactgggatcctttttcttgtggggcatggccagtcagatccttggagcagtacaggatg | |
| ttaatgcagaccgggaagctaatctgagctcaattgccactgtaattggggcgcgtggagccattcggctatcagta | |
| gtactttatttactagctgctgttttagtcactactttgcctaatccggcgtggatcatcgggattgcgattctaacttacgtat | |
| ttgatgccgcacgattttggaacattacagatgccagttgtgaacaggctaatcgcagttggaaagttttcctgtggctg | |
| aactactttgttggtgctgtgataacgatactgttaatagcaattcatcagatataa | |
| SEQ ID NO: 8 | |
| M. luteus NCTC2665 crtE2 polypeptide sequence | |
| MIRTLFWVSRPVSWVNTAYPFAAAAILTGGLPAWLVVLGVVFFLVPYNLAMYGIND | |
| VFDFASDLRNPRKGGVEGSVLGDPAVRRRVLAWSVLLPVPFVAVLAGWSAVRGE | |
| WAAVLVLAVSLFAVVAYSWAGLRFKERPFLDAATSATHFVSPAVYGLALAGATPT | |
| PALAALLGAFFLWGMASQMFGAVQDVVPDREGGLASVATVLGARRTVLLAAGLY | |
| AAAGLLLLATDPPGPLAALLAVPYVVNTLRFRRITDATSGAAHRGWQLFLPLNYVT | |
| GFLVTLLLIGWALTRGAAA | |
| SEQ ID NO: 9 | |
| C. glutamicum crtEb polypeptide sequence | |
| MMEKIRLILLSSRPISWVNTAYPFGLAYLLNAGEIDWLFWLGIVFFLIPYNIAMYGIN | |
| DVFDYESDIRNPRKGGVEGAVLPKSSHSTLLWASAISTIPFLVILFIFGTWMSSLWL | |
| TISVLAVIAYSAPKLRFKERPFIDALTSSTHFTSPALIGATITGTSPSAAMWIALGSFF | |
| LWGMASQILGAVQDVNADREANLSSIATVIGARGAIRLSVVLYLLAAVLVTTLPNPA | |
| WIIGIAILTYVFDAARFWNITDASCEQANRSWKVFLWLNYFVGAVITILLIAIHQI | |
| SEQ ID NO: 10 | |
| M. luteus Otnes 7 crtE2 nucleotide sequence | |
| atgatccgcaccctcttctgggcgtcccggccggtcagctgggtgaacacggcgtacccgttcgccgccgccgcga | |
| tcctgaccggggggctgcccgcgtggctggtggtcctgggcgtcgtgttcttcctcgtgccctacaacctggccatgta | |
| cggcatcaatgacgtgttcgacttcgcctcggacctgcgcaacccccgcaaggggggcgtggagggctccgtgct | |
| gggcgaccccgcggtgcgccgccgggtgctggtgtggtcggtgctgctgcccgtcccgttcgtggccgtgctcgcg | |
| ggctggtccgccgtgcggggcgagtgggccgccgtgctggtgctggcggtgagcctgttcgcggtggtggcgtact | |
| cctgggcggggctgcggttcaaggagcggcccttcctggacgccgcgacctccgccacccacttcgtctcccccgc | |
| ggtctacggcctcgtgctggccggggcgacccccacgcccgccctggcggcgctgctgggggccttcttcctgtgg | |
| ggcatggcctcgcagatgttcggggcggtgcaggacgtggtgccggaccgggaggggggcctggcctcggtggc | |
| caccgtgctgggcgctcggcgcaccgtcctgctcgccgccggcctgtacgcggcggcgggcctgctgctgctggc | |
| caccgacccgccgggcccccttgcggcgctgctggccgtgccctacgtggtgaacaccctgcgcttccgccgcatc | |
| acggacgccacctcgggcgcggcccaccgcggctggcagctgttcctccccctgaactacgtgaccggcttcctcg | |
| tgaccctgctgctgatcgggtgggcgctgacccggggggcggcggcatga | |
| SEQ ID NO: 11 | |
| M. luteus Otnes 7 crtE2 polypeptide sequence | |
| MIRTLFWASRPVSWVNTAYPFAAAAILTGGLPAWLVVLGVVFFLVPYNLAMYGIND | |
| VFDFASDLRNPRKGGVEGSVLGDPAVRRRVLVWSVLLPVPFVAVLAGWSAVRGE | |
| WAAVLVLAVSLFAVVAYSWAGLRFKERPFLDAATSATHFVSPAVYGLVLAGATPT | |
| PALAALLGAFFLWGMASQMFGAVQDVVPDREGGLASVATVLGARRTVLLAAGLY | |
| AAAGLLLLATDPPGPLAALLAVPYVVNTLRFRRITDATSGAAHRGWQLFLPLNYVT | |
| GFLVTLLLIGWALTRGAAA | |
| SEQ ID NO: 12 | |
| M. luteus Otnes 7 crtYg nucleotide sequence | |
| atgatctacctgctggccctgctgggtgtcatcggctgcatgctgctggtggaccggcgcttcgagctgttcctgtggca | |
| tcgcccgctcccggcgctgctggtgctggccgccggggtggcctacttcgtcgcctgggacctgtgggggatcgccg | |
| aaggcgtgttcctgcaccggcagtcgccctacgtgaccggggtgatgctcgccccccagctgcccctggaggagg | |
| ggttcttcctgctcttcctcagccagatcacgatggtgctgttcaccggggcgctgcgcctgctgcgcggccggggac | |
| gcgacgcccgtgccgcgacgccggccgatccgaccgacggggggagccggtga | |
| SEQ ID NO: 13 | |
| M. luteus Otnes 7 crtYg polypeptide sequence | |
| MIYLLALLGVIGCMLLVDRRFELFLWHRPLPALLVLAAGVAYFVAWDLWGIAEGVF | |
| LHRQSPYVTGVMLAPQLPLEEGFFLLFLSQITMVLFTGALRLLRGRGRDARAATPA | |
| DPTDGGSR | |
| SEQ ID NO: 14 | |
| M. luteus Otnes 7 crtYh nucleotide sequence | |
| gtgaccttcctcgacctcgtcctcgtcttcgtgggcttcgccctggccgtgctcgtgggcgccgccctcgtcggccgcgt | |
| gcggggcgagcacctgcgggccgtggcggccaccctggtggccctgtgggccctcacggcggtcttcgacaacg | |
| tgatgatcgccgcggggctcttcgactacggccatgagctgctggtgggtgcctacgtgggccaggcgcccgtgga | |
| ggacttcgcctacccgctcggctccgccctgctgctgccggcgctctggctgctgctgacgagccgtggtcgtgccg | |
| gtcggcgcggccctcggccgggacgccgcccccacccggacgatcgctga | |
| SEQ ID NO: 15 | |
| M. luteus Otnes 7 crtYh polypeptide sequence | |
| VTFLDLVLVFVGFALAVLVGAALVGRVRGEHLRAVAATLVALWALTAVFDNVMIAA | |
| GLFDYGHELLVGAYVGQAPVEDFAYPLGSALLLPALWLLLTSRGRAGRRGPRPG | |
| RRPHPDDR | |
| SEQ ID NO: 16 | |
| M. luteus NCTC2665 crtX nucleotide sequence | |
| gtgaccccggcccgccccacggtctccgtggtcgtcccggtgctcgacgacgccgagcacctgcgcgtgtgcctcg | |
| cgctgctggccgcccagagccggccggcgctggaggtggtggtggtggacaacggctgcgtggacgactcggc | |
| ggtgctcgcccgcgccgccggcgcgcgggtggtgcgcgagccgcgccgcggggtcccggccgcggcggccgc | |
| cggcctggacgccgcggtcggggagctgctggtgcgctgcgacgccgacacgcggatgcccgcggactggctc | |
| gaacggatcgtggcccggttcgacgccgaccccgggctcgacgccctcaccgggccggggaccttccacgacc | |
| agcccggcctccggggacaggtgcgggcggcgctctacaccggcacgtaccgctggggggcgggcgccgcgg | |
| tggcggccacccccgtctggggctccaactgcgccctgcgcgccgaggcgtggcaggctgtgcggacccgcgtc | |
| caccgcgaacgcggggacgtgcacgatgacctggacctgtccttccagctggccctggccggccgccggatccg | |
| gttcgatccggacctgcgggtggaggtcgccgggcgcatcttccactccctgcgccagcgggtgcggcagggccg | |
| gatggcggtcaccaccctgcaggtcaactgggcccgactgtcccccgggcggcgttggctgcgccgggcggccc | |
| gggcacacccccggtcccgctgggggcgtggccccgacggtcagtcccgggactga | |
| SEQ ID NO: 17 | |
| M. luteus NCTC2665 CrtX polypeptide sequence | |
| VTPARPTVSVVVPVLDDAEHLRVCLALLAAQSRPALEVVVVDNGCVDDSAVLARA | |
| AGARVVREPRRGVPAAAAAGLDAAVGELLVRCDADTRMPADWLERIVARFDADP | |
| GLDALTGPGTFHDQPGLRGQVRAALYTGTYRWGAGAAVAATPVWGSNCALRAE | |
| AWQAVRTRVHRERGDVHDDLDLSFQLALAGRRIRFDPDLRVEVAGRIFHSLRQRV | |
| RQGRMAVTTLQVNWARLSPGRRWLRRAARAHPRSRWGRGPDGQSRD | |
| SEQ ID NO: 18 | |
| M. luteus NCTC2665 crtE nucleotide sequence | |
| atgggtgaagcgaggacgggcggcgaggccgcgctctccggggtgaccgccgagctggacgccgcgctccga | |
| cacgccgcggcccaggcgcccggatccgccgccttcgccgagctgctcgactcgctccacgtccatgtgggcgcc | |
| ggcaagctcatccgcccccgtctcgtcgagctcggctggcgcctggcgaccgccgacccggtccctccgtccggc | |
| cgcgctgccgtcgaccgactcggggccgccttcgaactgctgcacaccgcgctgctcgtccacgacgacgtcatc | |
| gatcgggacgtgctgcggcgcggccagcccgccgtgcacgcctccgcccggcaccgcctcgaggcccgcggg | |
| gtgcccgccgcggacgccgcccacgccggggtcgccgtcgccctcatcgcgggggacgtcctgctcacccaggc | |
| gttccggctcgccgccacctgtgccgccgacaccgcccgggccgccgaggccgccgccgtcgtcttcgacgccg | |
| ccgccgtgactgcggccggcgagctcgaggacgtgctcctggggctgtcccgccacaccggtgaggagcccgat | |
| cccgaccgcatcctcgccatgcaacggctcaagacggcgcactacacggtcggcgcgcccctgcgcgccggcg | |
| ccctcctggccggggcggatcccgacctcgcccgggcgatgggcgaggccggcgccgacctcggcgccgccta | |
| ccaggtgatcgacgacgtcctcggcgtgttcggcgatcccggggagaccggcaagtccgccgacggcgacctgc | |
| gcgagggcaaggccaccgtgctcaccgcccacggccgccgcatccccgccgtccgcgccctgctcgacgcggg | |
| cccggccacccccgcggacatcgaggccgcccgccgcgccctcgaggcggccggtgcccgggagcacgccct | |
| cgacgtcgccgccgagctcaccgtccgcgcccgcgagcgcatcgcggccctgcccctggacgagacggtccgg | |
| gcggagttcgccgacgcctgccacgccgtgctgacccggaggtcctga | |
| SEQ ID NO: 19 | |
| M. luteus NCTC2665 crtE polypeptide sequence | |
| MGEARTGGEAALSGVTAELDAALRHAAAQAPGSAAFAELLDSLHVHVGAGKLIRP | |
| RLVELGWRLATADPVPPSGRAAVDRLGAAFELLHTALLVHDDVIDRDVLRRGQPA | |
| VHASARHRLEARGVPAADAAHAGVAVALIAGDVLLTQAFRLAATCAADTARAAEA | |
| AAVVFDAAAVTAAGELEDVLLGLSRHTGEEPDPDRILAMQRLKTAHYTVGAPLRA | |
| GALLAGADPDLARAMGEAGADLGAAYQVIDDVLGVFGDPGETGKSADGDLREGK | |
| ATVLTAHGRRIPAVRALLDAGPATPADIEAARRALEAAGAREHALDVAAELTVRAR | |
| ERIAALPLDETVRAEFADACHAVLTRRS | |
| SEQ ID NO: 20 | |
| M. luteus NCTC2665 crtB nucleotide sequence | |
| atggccgcgcccaccccgagccctgccgcgctgtacacgcggacggcccacaccgcagcggcccaggtgatcc | |
| gccgctactccacgtccttctcctgggcctgccgcaccctgccccggcaggcacgccaggacgtggccacgatcta | |
| cgccatggtccgcgtcgccgacgaggtggtcgacggcgtcgcggtggccgccgggctcgacgaggccggggtc | |
| cgcgccgccctggacgactacgagcgggcgtgtgaggccgcgatggcgtcgggcttcgccaccgacccggtcct | |
| gcacgccttcgccgacgtggcccgtcgccacggcatcaccccggagctgacccgtcccttcttcgcctccatgcgc | |
| gcggacctggggatccgcgagcacggcgccgagtccctggacgcctacatccacggctcggccgaggtggtgg | |
| ggctgatgtgcctgcaggtcttcctctccctccccggcacgcgggcccggaccccgggccagcggcaggagctgc | |
| gcgcgcaggcctcccggctgggggcggcgttccagaaggtcaacttcctcagggacctggccgcggaccaccac | |
| gagctgggccgcacctacctgcccggtgccgcaccgggcgtgctcaccgaggcccgcaaggccgagctcgtgg | |
| ccgaggtccgcgccgacctcgacgccgccctgcccggcatccgtgtcctggaccccggggccgggcgcgccgtg | |
| gccctggcgcacggactgttcgcggccctggtggaccggatcgaggcgaccccggcggccgagctggcccacc | |
| gccgtgtccgggtgccggaccatcagaaggcccggatcgccgcccgcgtcctggcacggggccgccggggag | |
| gccgccgatga | |
| SEQ ID NO: 21 | |
| M. luteus NCTC2665 crtB polypeptide sequence | |
| MAAPTPSPAALYTRTAHTAAAQVIRRYSTSFSWACRTLPRQARQDVATIYAMVRV | |
| ADEVVDGVAVAAGLDEAGVRAALDDYERACEAAMASGFATDPVLHAFADVARRH | |
| GITPELTRPFFASMRADLGIREHGAESLDAYIHGSAEVVGLMCLQVFLSLPGTRAR | |
| TPGQRQELRAQASRLGAAFQKVNFLRDLAADHHELGRTYLPGAAPGVLTEARKA | |
| ELVAEVRADLDAALPGIRVLDPGAGRAVALAHGLFAALVDRIEATPAAELAHRRVR | |
| VPDHQKARIAARVLARGRRGGRR | |
| SEQ ID NO: 22 | |
| M. luteus NCTC2665 crtl nucleotide sequence | |
| atgagcgcccgggacaccgctctcggcccgcgcaccgtggtggtgggcggcggtttcgccggactggccacggc | |
| gggcctgttggcccgcgacgggcaccgggtgacgctgctggagcgcggcgccgtcctgggcggccgtgccgga | |
| cgctggtccgaggcggggttcaccttcgataccgggccctcctggtacctgatgcccgaggtgatcgaccgctggtt | |
| ccgcctcatggggacctccgccgccgaacggctggacctgcgccgtctggaccccggctaccgggtgtacttcga | |
| ggggcacctccacgagccccccgtggacgtgcgcaccggccacgcggagacgctgttcgagtccctcgagccc | |
| ggcgccgggcgccggctgcgggcctacctcgactccgcgtcccggatctacgggctcgccaaggagcacttcctc | |
| tacacggacttccgccggccggccgccctggcccacccggacgtcctgcgcgccctgccggccctcgggcccca | |
| gctgctggggggcctgcgctcccacgtcgcggcccgcttccaggacccccggctgcgccagatcctgggctaccc | |
| ggcggtcttcctcggcacgtcccccgaccgtgcccccgccatgtaccacctgatgtcccatctggacctcgccgacg | |
| gcgtgcagtaccccctcggcgggttcgcggccctcgtggacgccatggcggaggtcgtgcgcgaggccggcgtg | |
| gagatccgcaccggggtcgaggcgaccgccgtggaggtcgcggaccgtcccgcccccgccggccgcctcgga | |
| cgcctggccgcccgcctgcccaggccgggagcagcccgcggggacgagggccgacgtcgccgcccgggccg | |
| ggtgaccggcgtcgcctggcggtccgacgacggcgccgcgggacgcctcgacgccgatgtggtggtggccgcc | |
| gcggacctgcaccacgtgcagacccgtctgctgcctcccggccggcgcgtcgcggagtccacgtgggaccggcg | |
| cgaccccggcccctccggcgtgctcgtgtgcgtgggggtgcgcggatccctgccccagctggcccatcacacctgc | |
| tgttcacggcggactgggaggacaacttcgggcgcatcgagcggggggaggacctcgccgcggacacgtcgat | |
| ctacgtctcgcgcacctccgccacggacccgggcgtggccccggagggcgacgagaacctcttcatcctcgtccc | |
| ggcccccgccgagccggggtgggggcgcggcggcatccgggtccgtgacggccagggctggcgggtggaccg | |
| cgccggggacgcccaggtggaggccgtggcggaccgggccctcgatcagctggcccgctgggccgggatcccc | |
| gacctggccgagcgcatcgtggtgcggcgcacctacgggcccggtgacttcgccgcggacgtgcacgcctggcg | |
| gggttcgctgctgggccccgggcacacgctggcgcagtcggccatgttccgcccctcggtgcgggacgcggacgt | |
| ggccggcctgatgtacgcgggctcctcggtgcgcccgggaatcggggtgcccatgtgcctgatctccgccgaagtg | |
| gtccgggacgaactgcgccacgacgcgcgcagggcccggcccgcgggccccggggggagcggcacatga | |
| SEQ ID NO: 23 | |
| M. luteus NCTC2665 crtl polypeptide sequence | |
| MSARDTALGPRTVVVGGGFAGLATAGLLARDGHRVTLLERGAVLGGRAGRWSE | |
| AGFTFDTGPSWYLMPEVIDRWFRLMGTSAAERLDLRRLDPGYRVYFEGHLHEPP | |
| VDVRTGHAETLFESLEPGAGRRLRAYLDSASRIYGLAKEHFLYTDFRRPAALAHPD | |
| VLRALPALGPQLLGGLRSHVAARFQDPRLRQILGYPAVFLGTSPDRAPAMYHLMS | |
| HLDLADGVQYPLGGFAALVDAMAEVVREAGVEIRTGVEATAVEVADRPAPAGRL | |
| GRLAARLPRPGAARGDEGRRRRPGRVTGVAWRSDDGAAGRLDADVVVAAADLH | |
| HVQTRLLPPGRRVAESTWDRRDPGPSGVLVCVGVRGSLPQLAHHTLLFTADWED | |
| NFGRIERGEDLAADTSIYVSRTSATDPGVAPEGDENLFILVPAPAEPGWGRGGIRV | |
| RDGQGWRVDRAGDAOVEAVADRALDOLARWAGIPDLAERIVVRRTYGPGDFAA | |
| DVHAWRGSLLGPGHTLAQSAMFRPSVRDADVAGLMYAGSSVRPGIGVPMCLISA | |
| EVVRDELRHDARRARPAGPGGSGT | |
| SEQ ID NO: 24 | |
| M. luteus NCTC2665 ORF1 nucleotide sequence | |
| gtgccgatcggcgcggccgtcggccgggacgccgcccccacccggacgatcgctgacatgctgccgttgatccc | |
| cgcagacctgctgcgcgcgctcggcctgatcctcgtcccggtcgcggcggtgcacgccggatggccgtccgcggc | |
| ggcgatgctgctcgtgttcggctcccagtggctcacccgctggctcgccccgggcggcgccctggactgggccgcg | |
| caggcggtcctgctgctggccgggtggctgagcgtcatcggcctctacccgcgggtgccgtggctggacctgctcgt | |
| gcacgccgccgcctccgccgtggtcgcctgtctgacggcactggtggtgggggcgtggctccggcgtcgggggac | |
| cgaggccgggcaggccgtggcgctgctcggcccgggcctggccgggctggggatcgcggccgccgccgtggcc | |
| ctgggcgtggtgtgggagctggccgaatggtgggggcacacggcggtgaccccggagatcggcgtgggctacac | |
| ggacaccatcggcgacctcgccgccgatctcgtcggcgccggggtcggcgccgccctcgccgtgtgccgggggc | |
| gcacccggtga | |
| SEQ ID NO: 25 | |
| M. luteus NCTC2665 ORF1 polypeptide sequence | |
| VPIGAAVGRDAAPTRTIADMLPLIPADLLRALGLILVPVAAVHAGWPSAAAMLLVFG | |
| SQWLTRWLAPGGALDWAAQAVLLLAGWLSVIGLYPRVPWLDLLVHAAASAVVAC | |
| LTALVVGAWLRRRGTEAGQAVALLGPGLAGLGIAAAAVALGVVWELAEWWGHTA | |
| VTPEIGVGYTDTIGDLAADLVGAGVGAALAVCRGRTR | |
| SEQ ID NO: 26 | |
| M. luteus Otnes 7 Sarcinaxanthin gene cluster | |
| 1 | atgggtgaag cgaggacggg cggcgaggcc gcgctctccg gggtgaccgc cgagctggac |
| 61 | gccgcgctcc gacatgccgc ggcccaggca cccggatccg ccgccttcgc cgagctgctc |
| 121 | gactcgctcc acgtccatgt gggcgccggc aagctcatcc gcccccgtct cgtcgagctc |
| 181 | ggctggcgcc tggcgaccgc cgacccggtc cctccgtccg gccgcgctgc cgtcgaccga |
| 241 | ctcggggccg ccttcgaact gctgcacacc gcgctgctcg tccacgacga cgtcatcgat |
| 301 | cgggacgtgc tgcggcgcgg ccagcccgcc gtgcacgcct ccgcccggca ccgcctcgag |
| 361 | gcccgcgggg tgcccgccgc ggacgccgcc cacgccgggg tcgccgtcgc cctcatcgcg |
| 421 | ggggacgtcc tgctcaccca ggcgttccgg ctcgccgcca cctgtgccgc cgacaccgcc |
| 481 | cgggccgccg aggccgccgc cgtcgtcttc gacgccgccg ccgtgaccgc ggccggcgag |
| 541 | ctcgaagacg tgctcctggg gctgtcccgc cacaccggtg aggagcccga tcccgaccgc |
| 601 | atcctcgcca tgcaacggct caagacggcg cactacacgg tcggcgcgcc cctgcgcgcc |
| 661 | ggcgccctcc tggccggggc ggatcccgac ctcgcccggg cgatgggcga ggccggcgcc |
| 721 | gacctcggcg ccgcctacca ggtgatcgac gacgtcctcg gcgtgttcgg cgatcccggg |
| 781 | gagaccggca agtccgccga cggcgacctg cgcgagggca aggccaccgt gctcaccgcc |
| 841 | cacggccgcc tcatccccgc cgtccgcgcc ctgctcgacg cgggcccggc cacccccgcg |
| 901 | gacatcgagg ccgcccgccg cgccctcgag gcggccggtg cccgggagca cgccctcgac |
| 961 | gtcgccgccg agctcaccgt ccgcgcccgc gagcgcatcg cggccctgcc cctggacgag |
| 1021 | acggtccggg cggagttcgc cgacgcctgc cacgccgtgc tgacccggag gtcctgagat |
| 1081 | ggccgcgccc accccgagcc ctgccgcgct gtacacgcgg acggcccaca ccgcagcggc |
| 1141 | ccaggtgatc cgccgctact ccacgtcctt ctcctgggcc tgccgcaccc tgccccggca |
| 1201 | ggcacgccag gacgtggcca cgatctacgc catggtccgc gtcgccgacg aggtggtcga |
| 1261 | cggcgtcgcg gtggccgccg ggctcgacga ggccggggtc cgcgccgccc tggacgacta |
| 1321 | cgagcgggcg tgtgaggctg cgatggcgtc gggcttcgcc accgacccgg tcctgcacgc |
| 1381 | cttcgccgac gtggcccgtc gccacggcat caccccggag ctgacccgtc ccttcttcgc |
| 1441 | ctccatgcgc gcggacctgg ggatccgcga gcacggcgcc gagtcgctgg acgcctacat |
| 1501 | ccacggctcg gccgaggtgg tggggctgat gtgcctgcag gtcttcctct ccctccccgg |
| 1561 | cacgcgggcc cggaccccgg gccagcggca ggagctgcgc gcgcaggcct cccggctggg |
| 1621 | ggcggcgttc cagaaggtca acttcctcag ggacctggcc gcggaccacc acgagctggg |
| 1681 | ccgcacctac ctgcccggtg ccgcaccggg cgtgctcacc gaggcccgca aggccgagct |
| 1741 | cgtggccgag gtccgcgccg acctcgacgc cgccctgccc ggcatccgtg tcctggaccc |
| 1801 | cggggccggg cgcgccgtgg ccctggcgca cggactgttc gcggccctgg tggaccggat |
| 1861 | cgaggcgacc ccggcggccg agctggccca ccgccgtgtc cgggtgccgg accatcagaa |
| 1921 | ggcccggatc gccgcccgcg tcctggcacg gggccgccgg ggaggccgcc gatgagcgcc |
| 1981 | cgggacaccg ctctcggccc gcgcaccgtg gtggtgggcg gcggtttcgc cggactggcc |
| 2041 | acggcgggcc tgttggcccg cgacgggcac cgggtgacgc tgctggagcg cggcgccgtc |
| 2101 | ctgggcggcc gtgccggacg ctggtctgag gcggggttca ccttcgatac cgggccctcc |
| 2161 | tggtacctga tgcccgaggt gatcgaccgc tggttccgcc tcatggggac ctccgccgcc |
| 2221 | gaacggctgg acctgcgccg tctggacccc ggctaccggg tgtacttcga ggggcacctc |
| 2281 | cacgagcccc ccgtggacgt gcgcaccggc cacgcggaga cgctgttcga gtccctcgag |
| 2341 | cccggcgccg ggcgccggct gcgggcctac ctcgactccg cgtcccggat ctacgggctc |
| 2401 | gccaaggagc acttcctcta cacggacttc cgccggccgg ccgccctggc ccacccggac |
| 2461 | gtcctgcgcg ccctgccggc cctcgggccc cagctgctgg ggggcctgcg ctcccacgtg |
| 2521 | gcggcccgct tccaggatcc ccggctgcgc cagatcctgg gctacccggc ggtcttcctc |
| 2581 | ggcacgtccc ccgaccgtgc ccccgccatg taccacctga tgtcccatct ggacctcgcc |
| 2641 | gacggcgtgc agtaccccct cggcgggttc gcggccctcg tggacgccat ggcggaggtc |
| 2701 | gtgcgcgagg ccggcgtgga gatccgcacc ggggtcgagg cgaccgccgt cgaggtggtg |
| 2761 | gaccgtcccg cccccgccgg ccgcctcgga cgcctggccg cccgcctgcc caggccggga |
| 2821 | gcagcccgcg gggacgaggg ccgacgtcgc cgcccgggcc aggtgaccgg cgtcgcctgg |
| 2881 | cggtccgacg acggcgccgc gggacgcctc gacgccgatg tggtggtggc cgccgcggac |
| 2941 | ctgcaccacg tgcagacccg tctgctgcct cccggccggc gcgtcgcgga gtccacgtgg |
| 3001 | gaccggcgcg accccggccc ctccggcgtg ctcgtgtgcg tgggggtgcg cggatccctg |
| 3061 | ccccagctgg cccatcacac cctgctgttc acggcggact gggaggacaa cttcgggcgc |
| 3121 | atcgagcggg gagaggacct cgccgcggac acgtcgatct acgtctcgcg cacctccgcc |
| 3181 | acggacccgg gcgtggcccc ggagggcgac gagaacctct tcatcctcgt cccggccccc |
| 3241 | gccgagccgg ggtgggggcg cggcggcatc cgggtccgtg acggcgaggg ctggcgggtg |
| 3301 | gaccgcgccg gggacgccca ggtggaggcc gtggcggacc gggccctcga ccagctggcc |
| 3361 | cgctgggccg ggatcccgga cctggccgag cgcatcgtgg tgcggcgcac ctacgggccc |
| 3421 | ggtgacttcg ccgcggacgt gcacgcctgg cggggttcgc tgctgggccc cgggcacacg |
| 3481 | ctggcgcagt cggccatgtt ccgtccctcg gtgcgggacg cggacgtggc cggcctgatg |
| 3541 | tacgcgggct cctcggtgcg cccgggcatc ggggtgccca tgtgtctgat ctccgccgaa |
| 3601 | gtggtccggg acgaactgcg ccacgacgcg cgcagggccc ggcccgcggg ccccgggggg |
| 3661 | agcggcacat gatccgcacc ctcttctggg cgtcccggcc ggtcagctgg gtgaacacgg |
| 3721 | cgtacccgtt cgccgccgcc gcgatcctga ccggggggct gcccgcgtgg ctggtggtcc |
| 3781 | tgggcgtcgt gttcttcctc gtgccctaca acctggccat gtacggcatc aatgacgtgt |
| 3841 | tcgacttcgc ctcggacctg cgcaaccccc gcaagggggg cgtggagggc tccgtgctgg |
| 3901 | gcgaccccgc ggtgcgccgc cgggtgctgg tgtggtcggt gctgctgccc gtcccgttcg |
| 3961 | tggccgtgct cgcgggctgg tccgccgtgc ggggcgagtg ggccgccgtg ctggtgctgg |
| 4021 | cggtgagcct gttcgcggtg gtggcgtact cctgggcggg gctgcggttc aaggagcggc |
| 4081 | ccttcctgga cgccgcgacc tccgccaccc acttcgtctc ccccgcggtc tacggcctcg |
| 4141 | tgctggccgg ggcgaccccc acgcccgccc tggcggcgct gctgggggcc ttcttcctgt |
| 4201 | ggggcatggc ctcgcagatg ttcggggcgg tgcaggacgt ggtgccggac cgggaggggg |
| 4261 | gcctggcctc ggtggccacc gtgctgggcg ctcggcgcac cgtcctgctc gccgccggcc |
| 4321 | tgtacgcggc ggcgggcctg ctgctgc tg gccaccgacc cgccgggccc ccttgcggcg |
| 4381 | ctgctggccg tgccctacgt ggtgaacacc ctgcgcttcc gccgcatcac ggacgccacc |
| 4441 | tcgggcgcgg cccaccgcgg ctggcagctg ttcctccccc tgaactacgt gaccggcttc |
| 4501 | ctcgtgaccc tgctgctgat cgggtgggcg ctgacccggg gggcggcggc atgatctacc |
| 4561 | tgctggccct gctgggtgtc atcggctgca Igctgctggt ggaccggcgc ttcgagctgt |
| 4621 | tcctgtggca Icgcccgctc ccggcgctgc tggtgctggc cgccggggtg gcctacttcg |
| 4681 | tcgcctggga cctgtggggg atcgccgaag gcgtgttcct gcaccggcag tcgccctacg |
| 4741 | tgaccggggt gatgctcgcc ccccagctgc ccctggagga ggggttcttc ctgctcttcc |
| 4801 | tcagccagat cacgatggtg ctgttcaccg gggcgctgcg cctgctgcgc ggccggggac |
| 4861 | gcgacgcccg tgccgcgacg ccggccgatc cgaccgacgg ggggagccgg tgaccttcct |
| 4921 | cgacctcgtc ctcgtcttcg tgggcttcgc cctggccgtg ctcgtgggcg ccgccctcgt |
| 4981 | cggccgcgtg cggggcgagc acctgcgggc cgtggcggcc accctggtgg ccctgtgggc |
| 5041 | cctcacggcg gtcttcgaca acgtgatgat cgccgcgggg ctcttcgact acggccatga |
| 5101 | gctgctggtg ggtgcctacg tgggccaggc gcccgtggag gacttcgcct acccgctcgg |
| 5161 | ctccgccctg ctgctgccgg cgctctggct gctgctgacg agccgtggtc gtgccggtcg |
| 5221 | gcgcggccct cggccgggac gccgccccca cccggacgat cgctgagcgg ccgcaaaaaa |
| 5281 | atcactagtg cggccgcctg caggtcgacc atatgggaga gctcccaacg cgttggatgc |
| 5341 | atagcttgag tattctatag tgtcacctaa atagctggcg |

SEQ ID NO: 27
M. luteus Otnes 7 crtE nucleotide sequence
atgggtgaagcgaggacgggcggcgaggccgcgctctccggggtgaccgccgagctggacgccgcgctccga
catgccgcggcccaggcacccggatccgccgccttcgccgagctgctcgactcgctccacgtccatgtgggcgcc
ggcaagctcatccgcccccgtctcgtcgagctcggctggcgcctggcgaccgccgacccggtccctccgtccggc
cgcgctgccgtcgaccgactcggggccgccttcgaactgctgcacaccgcgctgctcgtccacgacgacgtcatc
gatcgggacgtgctgcggcgcggccagcccgccgtgcacgcctccgcccggcaccgcctcgaggcccgcggg
gtgcccgccgcggacgccgcccacgccggggtcgccgtcgccctcatcgcgggggacgtcctgctcacccaggc
gttccggctcgccgccacctgtgccgccgacaccgcccgggccgccgaggccgccgccgtcgtcttcgacgccg
ccgccgtgaccgcggccggcgagctcgaagacgtgctcctggggctgtcccgccacaccggtgaggagcccgat
cccgaccgcatcctcgccatgcaacggctcaagacggcgcactacacggtcggcgcgcccctgcgcgccggcg
ccctcctggccggggcggatcccgacctcgcccgggcgatgggcgaggccggcgccgacctcggcgccgccta
ccaggtgatcgacgacgtcctcggcgtgttcggcgatcccggggagaccggcaagtccgccgacggcgacctgc
gcgagggcaaggccaccgtgctcaccgcccacggccgcctcatccccgccgtccgcgccctgctcgacgcggg
cccggccacccccgcggacatcgaggccgcccgccgcgccctcgaggcggccggtgcccgggagcacgccct
cgacgtcgccgccgagctcaccgtccgcgcccgcgagcgcatcgcggccctgcccctggacgagacggtccgg
gcggagttcgccgacgcctgccacgccgtgctgacccggaggtcctga
SEQ ID NO: 28
M. luteus Otnes 7 crtE polypeptide sequence
MGEARTGGEAALSGVTAELDAALRHAAAQAPGSAAFAELLDSLHVHVGAGKLIRP
RLVELGWRLATADPVPPSGRAAVDRLGAAFELLHTALLVHDDVIDRDVLRRGQPA
VHASARHRLEARGVPAADAAHAGVAVALIAGDVLLTQAFRLAATCAADTARAAEA
AAVVFDAAAVTAAGELEDVLLGLSRHTGEEPDPDRILAMQRLKTAHYTVGAPLRA
GALLAGADPDLARAMGEAGADLGAAYQVIDDVLGVFGDPGETGKSADGDLREGK
ATVLTAHGRLIPAVRALLDAGPATPADIEAARRALEAAGAREHALDVAAELTVRAR
ERIAALPLDETVRAEFADACHAVLTRRS
SEQ ID NO: 29
M. luteus Otnes 7 crtB nucleotide sequence
atggccgcgcccaccccgagccctgccgcgctgtacacgcggacggcccacaccgcagcggcccaggtgatcc
gccgctactccacgtccttctcctgggcctgccgcaccctgccccggcaggcacgccaggacgtggccacgatcta
cgccatggtccgcgtcgccgacgaggtggtcgacggcgtcgcggtggccgccgggctcgacgaggccggggtc
cgcgccgccctggacgactacgagcgggcgtgtgaggctgcgatggcgtcgggcttcgccaccgacccggtcct
gcacgccttcgccgacgtggcccgtcgccacggcatcaccccggagctgacccgtcccttcttcgcctccatgcgc
gcggacctggggatccgcgagcacggcgccgagtcgctggacgcctacatccacggctcggccgaggtggtgg
ggctgatgtgcctgcaggtcttcctctccctccccggcacgcgggcccggaccccgggccagcggcaggagctgc
gcgcgcaggcctcccggctgggggcggcgttccagaaggtcaacttcctcagggacctggccgcggaccaccac
gagctgggccgcacctacctgcccggtgccgcaccgggcgtgctcaccgaggcccgcaaggccgagctcgtgg
ccgaggtccgcgccgacctcgacgccgccctgcccggcatccgtgtcctggaccccggggccgggcgcgccgtg
gccctggcgcacggactgttcgcggccctggtggaccggatcgaggcgaccccggcggccgagctggcccacc
gccgtgtccgggtgccggaccatcagaaggcccggatcgccgcccgcgtcctggcacggggccgccggggag
gccgccgatga
SEQ ID NO: 30
M. luteus Olnes 7 crtB polypeptide sequence
MAAPTPSPAALYTRTAHTAAAQVIRRYSTSFSWACRTLPRQARQDVATIYAMVRV
ADEVVDGVAVAAGLDEAGVRAALDDYERACEAAMASGFATDPVLHAFADVARRH
GITPELTRPFFASMRADLGIREHGAESLDAYIHGSAEVVGLMCLQVFLSLPGTRAR
TPGQRQELRAQASRLGAAFQKVNFLRDLAADHHELGRTYLPGAAPGVLTEARKA
ELVAEVRADLDAALPGIRVLDPGAGRAVALAHGLFAALVDRIEATPAAELAHRRVR
VPDHQKARIAARVLARGRRGGRR
SEQ ID NO: 31
M. luteus Otnes 7 crtl nucleotide sequence
atgagcgcccgggacaccgctctcggcccgcgcaccgtggtggtgggcggcggtttcgccggactggccacggc
gggcctgttggcccgcgacgggcaccgggtgacgctgctggagcgcggcgccgtcctgggcggccgtgccgga
cgctggtctgaggcggggttcaccttcgataccgggccctcctggtacctgatgcccgaggtgatcgaccgctggttc
cgcctcatggggacctccgccgccgaacggctggacctgcgccgtctggaccccggctaccgggtgtacttcgag
gggcacctccacgagccccccgtggacgtgcgcaccggccacgcggagacgctgttcgagtccctcgagcccg
gcgccgggcgccggctgcgggcctacctcgactccgcgtcccggatctacgggctcgccaaggagcacttcctct
acacggacttccgccggccggccgccctggcccacccggacgtcctgcgcgccctgccggccctcgggccccag
ctgctggggggcctgcgctcccacgtggcggcccgcttccaggatccccggctgcgccagatcctgggctacccg
gcggtcttcctcggcacgtcccccgaccgtgcccccgccatgtaccacctgatgtcccatctggacctcgccgacgg
cgtgcagtaccccctcggcgggttcgcggccctcgtggacgccatggcggaggtcgtgcgcgaggccggcgtgg
agatccgcaccggggtcgaggcgaccgccgtcgaggtggtggaccgtcccgcccccgccggccgcctcggacg
cctggccgcccgcctgcccaggccgggagcagcccgcggggacgagggccgacgtcgccgcccgggccagg
tgaccggcgtcgcctggcggtccgacgacggcgccgcgggacgcctcgacgccgatgtggtggtggccgccgc
ggacctgcaccacgtgcagacccgtctgctgcctcccggccggcgcgtcgcggagtccacgtgggaccggcgcg
accccggcccctccggcgtgctcgtgtgcgtgggggtgcgcggatccctgccccagctggcccatcacaccctgct
gttcacggcggactgggaggacaacttcgggcgcatcgagcggggagaggacctcgccgcggacacgtcgatc
tacgtctcgcgcacctccgccacggacccgggcgtggccccggagggcgacgagaacctcttcatcctcgtcccg
gcccccgccgagccggggtgggggcgcggcggcatccgggtccgtgacggcgagggctggcgggtggaccgc
gccggggacgcccaggtggaggccgtggcggaccgggccctcgaccagctggcccgctgggccgggatcccg
gacctggccgagcgcatcgtggtgcggcgcacctacgggcccggtgacttcgccgcggacgtgcacgcctggcg
gggttcgctgctgggccccgggcacacgctggcgcagtcggccatgttccgtccctcggtgcgggacgcggacgt
ggccggcctgatgtacgcgggctcctcggtgcgcccgggcatcggggtgcccatgtgtctgatctccgccgaagtg
gtccgggacgaactgcgccacgacgcgcgcagggcccggcccgcgggccccggggggagcggcacatga
SEQ ID NO: 32
M. luteus Otnes 7 crtl polypeptide sequence
MSARDTALGPRTVVVGGGFAGLATAGLLARDGHRVTLLERGAVLGGRAGRWSE
AGFTFDTGPSWYLMPEVIDRWFRLMGTSAAERLDLRRLDPGYRVYFEGHLHEPP
VDVRTGHAETLFESLEPGAGRRLRAYLDSASRIYGLAKEHFLYTDFRRPAALAHPD
VLRALPALGPQLLGGLRSHVAARFQDPRLRQILGYPAVFLGTSPDRAPAMYHLMS
HLDLADGVQYPLGGFAALVDAMAEVVREAGVEIRTGVEATAVEVVDRPAPAGRL
GRLAARLPRPGAARGDEGRRRRPGQVTGVAWRSDDGAAGRLDADVVVAAADLH
HVQTRLLPPGRRVAESTWDRRDPGPSGVLVCVGVRGSLPQLAHHTLLFTADWED
NFGRIERGEDLAADTSIYVSRTSATDPGVAPEGDENLFILVPAPAEPGWGRGGIRV
RDGEGWRVDRAGDAQVEAVADRALDQLARWAGIPDLAERIVVRRTYGPGDFAA
DVHAWRGSLLGPGHTLAQSAMFRPSVRDADVAGLMYAGSSVRPGIGVPMCLISA
EVVRDELRHDARRARPAGPGGSGT

### Example 1

### Formulations

Exemplary formulations in accordance with the invention are as follows:

### Sunscreens

### Body Lotions

| FORMULATION 1 | % w/w |
|---|---|
| Lanolin | 4.5 |
| Cocoa butter | 2.0 |
| Glyceryl stearate | 3.0 |
| Stearic acid | 2.0 |
| Octyl dimethyl PABA (UVB filter, optional) | 7.0 |
| Benzophenone-3 (UVB filter, optional) | 3.0 |
| Propylparaben | 0.1 |
| Methylparaben | 0.3 |
| Triethanolamine | 1.0 |
| Sorbitol | 5.0 |
| Carotenoid of the invention | 1.0-5.0 |
| Water | qs to 100 |

| FORMULATION 2 | % w/w |
|---|---|
| Phase A | |
| Isopropyl myristate | 4.0 |
| Mineral oil | 6.5 |
| Grape seed oil | 2.5 |
| Stearyl alcohol | 2.0 |
| Petrolatum | 2.0 |
| Octyl methoxycinnamate (UVB filter- | |
| optional) | 5.0 |
| Carotenoid of the invention | 1.0-5.0 |
| Phase B | |
| Sorbitan stearate | 6.0 |
| Disodium ricinoleamido MEA-sulfosuccinate | 0.2 |
| Glycerine | 4.0 |
| Allantoin | 0.2 |
| d-Panthenol | 0.8 |
| titanium oxide and water (optional) | 15.0 |
| Water | qs to 100 |
| | (phase A&B) |
| Phase C | |
| Preservative | qs |

Produced by separately heating phases A and B to 80° C., then adding A to B, stirring intensively. After homogenizing the mixture is allowed to cool to 25° C. with slow agitation after which phase C is added.

### Hair Products

| SHAMPOO | % w/w |
|---|---|
| Anionic surfactant | 2.5-1.5 active |
| Amphoteric surfactant | 0-4 active |
| Alkanolamide | 0-5 |
| Polymeric/associative thickener | 0-5 |
| Carotenoid of the invention | 1-5 |
| UVA/B filters (e.g octyl methoxy | 1-10 |
| cinnamate, avobenzone or | |
| oxybenzone)-optional | |
| Preservative | qs |
| Fragrance | qs |
| pH adjuster | qs |
| Electrolyte | qs |
| Water | qs to 100 |

| HAIRSPRAY | % w/w |
|---|---|
| Resin plasticizer | 0-2 |
| Film forming resin | 2-8 |
| Ethanol | 0-70 |
| Alkanolamine or alternative | 0-4 |
| neutralizing agent | |
| Carotenoid of the invention | 1-5 |
| UVA/B filters (e.g octyl methoxy | 1-10 |
| cinnamate, avobenzone or | |
| oxybenzone)-optional | |
| Preservative | qs |
| Fragrance | qs |
| Hydrocarbon or alternative propellant | 10-40 |
| Water | qs to 100 |

### Example 2

### Extraction/Preparation Protocols for Sarcinaxanthin

### General Background

The biosynthetic machinery responsible for the synthesis of sarcinaxanthin was unknown. A gene cluster for the biosynthesis of sarcinaxanthin which has not heretofore been available has been identified, cloned and sequenced. Furthermore, a novel strain of M. luteus, named Otnes 7, has been identified which is capable of producing sarcinaxanthin in superior quantities to other known strains. The identification, cloning and sequencing of the gene cluster for the biosynthesis of sarcinaxanthin from M. luteus strain NCTC2665 has allowed the identification and cloning of nucleic acids from the Otnes 7 strain, which encode novel proteins the expression of which results in increased sarcinaxanthin production in comparison to the proteins of the NCTC2665 strain. Heterologous expression of one or more of the sarcinaxanthin biosynthesis genes in a host cell has enabled a method for efficiently and economically producing sarcinaxanthin.

Since the chemical synthesis of compounds such as sarcinaxanthin is highly complex, a biosynthetic route in practice needs to be used and accordingly the isolation or purification of the compounds from appropriate hosts, particularly heterologous hosts (that is hosts transformed with one or more genes to enable the biosynthesis), is desirable. This also affords the opportunity of manipulating genes of the biosynthetic gene cluster in order to change the biosynthesis and thereby result in improved yields and/or the synthesis of new or modified carotenoid compounds.

Sarcinaxanthin has been isolated and purified from a previously unknown source, bacterial isolate Otnes 7, believed to be a novel strain of M. luteus (deposited in the name of the applicant under the deposit number DSM 23579, on 29 Apr. 2010, at the Deutsche Sammlung von Mikroorganismen and Zellkulturen GmbH (DSMZ)) which was isolated from surface micro layer of the mid-part of the Norwegian coast. The biosynthetic gene cluster contains 8 genes that encode proteins that are believed to be involved in the biosynthesis of the sarcinaxanthin molecule and derivatives thereof (see Table 1).

Based on the knowledge of the sequence, the inventors have been able to use various methods of genetic manipulation to confirm the activity of the proteins encoded by the gene cluster and to show that the sequences identified in the Otnes 7 strain are indeed responsible for enhanced sarcinaxanthin biosynthesis.

The complete coding sequence for (i.e. the complete nucleotide sequence encoding) the sarcinoxanthin biosynthetic gene cluster from the NCTC2665 strain is shown in SEQ ID NO. 1. This has been shown to contain a number of genes or ORFs, that are believed to encode all of the proteins and polypeptides that are required for normal sarcinaxanthin biosynthesis in M. luteus. The group of proteins and polypeptides encoded by the gene cluster as a whole are collectively referred to as the biosynthetic machinery for the biosynthesis of sarcinaxanthin.

In silico screening the of the M. luteus strain NCTC2665 DNA sequence data (which has been deposited under accession number NC_{—}012803) resulted in the initial identification of a putative carotenoid biosynthesis gene cluster consisting of six open reading frames, or1009-or1014 (comprised within SEQ ID NO: 1). The deduced or1014 gene product displayed only 31% and 33% primary sequence identity to known CrtE proteins of C. glutamicum and Dietzia sp., respectively, both encoding geranyl geranyl pyrophosphate (GGPP) synthases. CrtE catalyzes the first reaction specific to the carotenoid branch of general isoprenoid metabolism, the conversion of farnesyl pyrophosphate (FPP) into GGPP. The or1014 gene was therefore designated crtE (SEQ ID NO: 18 and 19). The deduced or1013 gene product displayed only 41% and 48% primary sequence identity to the CrtB proteins of C. glutamicum and Dietzia sp., respectively, which are phytoene synthases which catalyze the condensation of two GGPP molecules to phytoene. The or1013 gene was therefore designated crtB (SEQ ID NO: 20 and 21). The deduced or1012 gene product displayed only 43% and 53% primary sequence identity to the Crtl proteins of C. glutamicum and Dietzia sp., respectively. These proteins are phytoene desaturases which catalyse conversion of phytoene to lycopene by stepwise desaturation reactions. The or1012 gene was therefore designated crtl (SEQ ID NO: 22 and 23). The deduced or1011 gene product displayed only 50% and 52% primary sequence identity to the lycopene elongases in C. glutamicum and in Dietzia sp., respectively. In C. glutamicum this enzyme (encoded by crtEb) catalyses the conversion of lycopene into nonaflavuxanthin and flavuxanthin. Secondary structure analysis revealed six transmembrane helices for the M. luteus elongase, five for the C. glutamicum elongase and eight for the Dietzia sp. elongase, strongly indicating that all are transmembrane proteins. The or1011 gene was designated crtE2 (SEQ ID NO: 6 and 8). The deduced or1010 and or1009 gene products displayed only 32% and 31% primary sequence identity to the C₅₀ ε-cyclase subunits in C. glutamicum encoded by crtYe and crtYf, respectively. They also shared only 36% and 38% primary sequence identity to the corresponding proteins in Dietzia sp. In C. glutamicum, the crtYe and crtYf gene products are small polypeptides assumed to form a heterodimeric enzyme that catalyses the conversion of flavuxanthin into decaprenoxanthin. Both gene products exhibit three transmembrane helices. Secondary structure analysis revealed also three transmembrane helices for each C₅₀ cyclase subunit from C. glutamicum and Dietzia sp. The or1010 and or1009 genes were designated crtYg (SEQ ID NO: 2 and 3) and crtYh (SEQ ID NO: 4 and 5), respectively.

Further analysis of the gene cluster revealed that immediately downstream of crtYh there is a an ORF encoding a hypothetical protein (SEQ ID NO: 24 and 25), followed by or1007 which encodes a putative polypeptide sharing only 43% sequence identity to the putative glycosyl transferase protein CrtX from Dietzia sp., suggested to be involved in the glycosylation of C.p.450 (Tao et al., 2007). The or 1007 gene was therefore designated crtX (SEQ ID NO: 16 and 17).

Without wishing to be bound by any single hypothesis, it is believed, due to the proximal localization and similar orientation of the genes, that the crtEIBE2YgYh genes are cotranscribed in M. luteus. Moreover, the assumed stop codons of crtB, crtl, crtE2 and crtYg overlap the start codon of the corresponding subsequent gene which may allow translational coupling to ensure equimolar expression and/or proper folding of the products. Whilst the genetic organization of crt genes in M. luteus displays some similarities to the previously published biosynthetic gene clusters for the C₅₀ carotenoids C.p.450 and decaprenoxanthin in Dietzia sp., in view of the differences in the order of the genes and the relatively low sequence identity between the genes it was only after experimental analysis, as discussed elsewhere herein, that the above described gene cluster was confirmed as being involved in sarcinaxanthin biosynthesis.

As discussed above, the sarcinaxanthin biosynthetic gene cluster is a nucleic acid molecule which contains the various genetic elements or different genes or ORFs that encode the proteins or polypeptides that are required for the biosynthesis of the sarcinaxanthin molecule or a sarcinaxanthin derivative. However, not all of the encoded proteins and polypeptides have yet been ascribed a role in the biosynthesis and so it is thought that not all of the encoded proteins or polypeptides of the cluster are essential for sarcinaxanthin biosynthesis. The various genes and ORFs may encode enzymes that catalyse one or more biochemical reactions, or proteins that do not have catalytic activity but instead are involved in other processes such as the regulation of the process of sarcinaxanthin synthesis, or sarxinaxanthin transport, for example.

Each sarcinaxanthin biosynthetic gene or ORF encodes a single polypeptide chain that has or is believed to have a function in the biosynthesis of the sarcinaxanthin molecule or a derivative thereof. Eight such genes or ORFs have been identified (see Table 1). As shown in FIG. 3, six of these are ascribed a direct role in the biosynthesis of sarcinaxanthin, whilst a seventh has been shown to have a role in the glycosylation of sarcinaxanthin to mono- and diglucoside forms and the eighth has not yet been ascribed a function.

However, as discussed further below, only two of the genes or ORFs are essential for the biosynthesis of sarcinaxanthin, i.e. those encoding the enzyme which catalyses the final step of the biosynthetic pathway that results in the conversion of flavuxanthin to sarcinaxanthin (namely crtYg and crtYh) and the other genes may be replaced by genes encoding enzymes with equivalent functional activities, or alternative activities that result in the production of flavuxanthin, i.e. the substrate for the C₅₀ carotenoid γ-cyclase encoded by said genes. In other words, for the production of sarcinaxanthin in a host cell it is not necessary to introduce into said cell the entire biosynthetic cluster from M. luteus as the introduction of genes encoding the enzymes that catalyse the final step in the biosynthetic pathway is sufficient for the production of sarcinaxanthin as long as the substrate for the sarcinxanthin-synthesising C₅₀ carotenoid γ-cyclase, i.e. flavuxanthin, is present in said cell.

In particular, as described in the example below, it has been found that higher levels of sarcinaxanthin production may be obtained by recombinant expression of the sarcinaxanthin-producing enzymes (i.e. of the sarcinaxanthin biosynthetic machinery) in a heterologous host, as compared with sarcinaxanthin production in native M. luteus cells. Thus, in terms of sarcinaxanthin production, recombinant expression is favoured over extraction from natural sources (i.e. over isolation of the product from cells in which it is naturally produced).

Thus in a very general sense, sarcinaxanthin or a derivative thereof may be produced by introducing into and expressing in a host cell one or more nucleic acid molecules encoding the sarcinaxanthin biosynthetic pathway. By allowing the nucleic acid molecules to be expressed, the encoded biosynthetic machinery may act in the host cell to synthesise the sarcinaxanthin, which may be recovered from the host cell using the extraction procedure described below or other known suitable methods for extracting carotenoids. Thus, the sarcinaxanthin or derivative thereof is synthesised in the host cell and then isolated from the host cell.

As noted above, it is not necessary to introduce the entire biosynthetic pathway into the host, as long as the host is capable of making an intermediate, or substrate in the pathway (i.e. a sarcinaxanthin precursor). For example, a host already capable of synthesising lycopene, and/or flavuxanthin, may be used.

As noted above, such a host cell will be a cell which produces an appropriate substrate or substrates for the introduced activity or activities, for example a lycopene-producing host cell, or a flavuxanthin-producing host cell. Preferably the host cells do not endogenously contain all of the nucleic acid molecules required for the synthesis of sarcinaxanthin or a derivative thereof, i.e. do not naturally produce sarcinaxanthin, but may preferably comprise nucleic acid molecules encoding proteins required for the synthesis of sarcinaxanthin precursors, e.g. lycopene, nonaflavuxanthin or flavuxanthin. Such nucleic acid molecules may be present endogenously i.e. the host cell may be a native producer of lycopene, nonaflavuxanthin and/or flavuxanthin. Preferably the host cell is a cell or microorganism other than that from which the nucleic acid molecules were (or from which they may be) derived and in which the molecules are natively present.

As will be described in more detail below, the nucleic acid molecules which are introduced will preferably encode one or more of the biosynthetic proteins of the organism M. luteus. In other words the nucleic acid molecules will be derived from, or will correspond to, the crt genes of M. luteus, as described herein. As noted above, and described in more detail below, in certain cases, for example in case of proteins involved in the biosynthesis up to the intermediate flavuxanthin, nucleic acid molecules encoding equivalent proteins from other sources may be used.

More particularly, the method of the invention involves (or comprises) the introduction and expression of a nucleic acid molecule encoding a protein having C₅₀ carotenoid γ-cyclase activity. Such a protein may be an enzyme which catalyses the conversion of flavuxanthin to sarcinaxanthin, and in particular such an enzyme which performs this reaction in M. luteus. Thus, the protein may correspond to the gene product of the crtYgYh genes of M. luteus. Such proteins are described further below.

As noted above, the gene cluster for the entire biosynthetic pathway for sarcinaxanthin has been cloned and identified in M. luteus. Whilst a nucleic acid molecule corresponding to the entire gene cluster of M. luteus may be used to generate sarcinaxanthin, nucleic acid molecules based on genes encoding equivalent proteins from other sources may be used to provide the host cell with the proteins needed to synthesize a substrate, or intermediate, in the pathway. Thus for example host cells producing lycopene are known in the art, as are nucleic acid molecules encoding lycopene-synthesising enzymes, which may be used to engineer a host cell suitable for use, to produce lycopene. Similarly a flavuxanthin-producing host cell may be used, or may be engineered to produce flavuxanthin.

More specific embodiments are described further below. However, in general terms nucleic acid molecules may be obtained or derived from M. luteus, e.g. they may correspond to or be derived from the nucleotide sequences from M. luteus encoding proteins having or contributing to C₅₀ carotenoid γ-cyclase activity, as described herein, more particularly they may be correspond to or be derived from the crtYg or crtYh genes of M. luteus as described herein. The nucleic acid molecules encoding proteins capable of synthesising flavuxanthin may be obtained or derived from other sources, for example from genes known to be efficient in encoding proteins for lycopene synthesis in other organisms (e.g. the crtEIB genes from Pantoea ananatis, which are particularly useful in this respect, are described below), and by way of further example, nucleic acid molecules encoding proteins having lycopene elongase activity may be obtained or derived from organisms synthesising flavuxanthin, such as Corynebacterium glutamicum (crtEb) or from M. luteus (crtE2).

Thus, in general sarcinaxanthin may be generated by introducing into and expressing in a host cell one or more nucleic acid molecules comprising a nucleotide sequence encoding:

(i) a protein capable of catalysing the conversion of farnesyl pyrophosphate (FPP) into geranyl geranyl pyrophosphate (GGPP) (e.g. a protein as encoded by a crtE gene);

(ii) a protein capable of catalysing the condensation of GGPP to phytoene (e.g. a protein as encoded by a crtB gene);

(iii) a protein capable of catalysing the conversion of phytoene to lycopene, or alternatively put a protein having phytoene dehydrogenase activity (e.g. a protein as encoded by a crtl gene);

(iv) a protein capable of catalysing the conversion of lycopene to flavuxanthin, or, alternatively viewed, having lycopene elongase activity (e.g. a protein as encoded by a crtE2 or a crtEb gene); and

(v) a protein having or contributing to C₅₀ carotenoid γ-cyclase activity, or, alternatively viewed, capable of catalysing the conversion of flavuxanthin to sarcinaxanthin (e.g. proteins as encoded by a crtYg gene and a crtYh gene as described herein).

As noted above, preferably nucleic acid molecules encoding (iv) and (v) above are introduced into lycopene-producing host.

By way of representative example, the method may comprise introducing into a host cell and expressing a nucleic acid molecule comprising the nucleotide sequence encoding the entire biosynthetic gene cluster, for example as obtained or derivable from a strain of M. luteus, e.g. as set forth in SEQ ID NO: 1 or SEQ ID NO: 26 or a sequence with at least 70% sequence identity to SEQ ID NO: 1 or 26, or a part thereof, including particularly a part encoding the sarcinaxanthin biosynthetic pathway. Such a molecule may include a part of SEQ ID NO:1 or 26 which encodes one or more activities in the biosynthetic pathway, and more particularly a part which encodes a C₅₀ carotenoid γ-cyclase activity.

The nucleic acid molecules for use in the method need not comprise the entire sarcinaxanthin biosynthetic gene cluster but may comprise a portion or part of it, more specifically a part encoding one or more proteins having a particular enzymic activity, and particularly a C₅₀ carotenoid γ-cyclase activity, more particularly a lycopene elongase activity and a C₅₀ carotenoid γ-cyclase activity.

As mentioned above, a number of genes and ORFs have been identified within SEQ ID NO:1 and SEQ ID NO: 26 and parts or fragments which correspond to such genes or ORFs represent preferred “parts” or fragments of SEQ ID NO:1 or 26. These are tabulated in Table 1 below:

TABLE 1

| | | | | SEQ ID NO: |
|---|---|---|---|---|
| | Start position | End position | Function of | (nucleic |
| | in SEQ ID | in SEQ ID | encoded | acid/ |
| Name | NO: 1 (bp) | NO: 1 (bp) | protein | protein) |
| crtE | 561 | 1637 | Geranyl geranyl | 18/19 |
| | | | pyrophosphatase | |
| | | | (GGPP) | |
| crtB | 1639 | 2535 | Phytoene synthase | 20/21 |
| crtI | 2532 | 4232 | Phytoene desaturase | 22/23 |
| crtE2 | 4229 | 5113 | Lycopene elongase | 6/8 |
| crtYg | 5110 | 5472 | C₅₀ γ-cyclase subunit | 2/3 |
| crtYh | 5469 | 5822 | C₅₀ γ-cyclase subunit | 4/5 |
| ORF1 | 5767 | 6375 | Hypothetical protein | 24/25 |
| crtX | 6372 | 7163 | Sarcinaxanthin | 16/17 |
| | | | glycosylase | |
| | | | | SEQ ID NO: |
| | Start position | End position | Function of | (nucleic |
| | in SEQ ID | in SEQ ID | encoded | acid/ |
| Name | NO: 26 (bp) | NO: 26 (bp) | protein | protein) |
| crtE | 1 | 1077 | Geranyl geranyl | 27/28 |
| | | | pyrophosphatase | |
| | | | (GGPP) | |
| crtB | 1079 | 1975 | Phytoene synthase | 29/30 |
| crtI | 1972 | 3672 | Phytoene desaturase | 31/32 |
| crtE2 | 3669 | 4553 | Lycopene elongase | 10/11 |
| crtYg | 4550 | 4912 | C₅₀ γ-cyclase subunit | 12/13 |
| crtYh | 4909 | 5265 | C₅₀ γ-cyclase subunit | 14/15 |

The sequences set out above thus represent sarcinaxanthin biosynthetic genes or ORFs.

The sarcinaxanthin biosynthetic gene cluster has also been cloned from the novel Micrococcus luteus strain Otnes 7, and the proteins encoded by said genes can be considered as functional equivalents of the NCTC2665 sarcinaxanthin biosynthetic proteins. However, as discussed below, the Otnes 7 strain produces increased levels of carotenoids in comparison to the NCTC2665 strain, e.g. 190 μg/g cell dry weight (CDW) and 145 μg/g CDW, respectively. This difference in sarcinaxanthin production is sufficient to distinguish between the two strains by visual inspection as the difference between colour intensities of the M. luteus strains demonstrates clearly that the Otnes 7 strain produces higher levels of sarcinaxanthin than the NCTC2665 strain. Furthermore, when expressed in a heterologous host, the Otnes 7 genes resulted in higher sarcinaxanthin production levels as compared to expression of the NCTC2665 genes. From experimental analysis of the Otnes 7 biosynthetic gene cluster it was determined that the Otnes 7 genes comprise specific sequence modifications as compared to the genes from the NCTC2665 strain. It is unclear exactly why the Otnes 7 genes result in increased production, and this may depend upon the host used for the expression. However, it is possible that they encode proteins which have an enhanced catalytic activity (or substrate conversion efficiency) in comparison to genes of the NCTC2665 strain. Specifically, in the experiments in the Example described below the crtE2 protein from the Otnes 7 strain shows a relative conversion efficiency of lycopene to nonaflavuxanthin and flavuxanthin of 79% in comparison to the equivalent protein from the NCTC2665 strain, which has a conversion efficiency of only 23%. Furthermore, when the nucleic acids from the Otnes 7 strain encoding crtE2, crtYg and crtYh are expressed in a heterologous host cell, at least 97% of the carotenoid produced was sarcinaxanthin, wherein the expression of the same genes from NCTC2665 resulted in only about 90% of the carotenoids produced being sarcinaxanthin.

Although the nucleic acids used in methods of producing sarcinaxanthin may correspond to native genes/ORFs or may encode native proteins, as noted above the respective nucleotide and/or amino acid sequences may be modified. The modification may take place by modifying one or more nucleotide sequences so as to cause the modification of one or more encoded proteins. This may result in alteration of enzyme activity e.g. improved enzymatic activity and consequently may enhance yields of sarcinaxanthin or derivatives thereof. Alternatively, such a modification may be desirable to facilitate the operation of the method, for example construction of an expression vector etc, or otherwise in the manipulation of the nucleic acids, or it may result in improved expression etc, or enable expression in a different host etc. Thus, by way of example, nucleic acid molecules of the invention may be utilised to manipulate or facilitate the biosynthetic process, for example by extending the host range or increasing yield or production efficiency etc.

A host may be used which already contains some of the genes required to make precursors in the sarcinaxanthin pathway, e.g. a lycopene-producing host cell. In such a host, modification of the genes which are already present in the host may take place in situ. In other words, in a lycopene-producing host for example, the endogenous genes already present for lycopene production may be altered, for example to increase lycopene production, e.g. by gene replacement, the introduction of new regulatory sequences or mutagenesis.

Thus, one method of producing sarcinaxanthin may comprise introducing into a lycopene-producing host cell and expressing:

(a) a nucleic acid molecules encoding a protein capable of catalysing the conversion of lycopene to flavuxanthin, or alternatively put a protein having lycopene elongase activity;

(b) a nucleic acid molecule encoding a C₅₀ carotenoid γ-cyclase subunit and comprising:
(i) a nucleotide sequence as set forth in all or part of SEQ ID NO: 2 or SEQ ID NO: 12, or which is degenerate therewith, or which has at least 70% sequence identity to SEQ ID NO: 2 or 12;
(ii) a nucleotide sequence which hybridizes to SEQ ID NO: 2 or 12 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 3 or 13 or an amino acid sequence which is at least 70% identical to SEQ ID NO: 3 or 13; and

(c) a nucleic acid molecule encoding a C₅₀ carotenoid γ-cyclase subunit and comprising:
(i) a nucleotide sequence as set forth in all or part of SEQ ID NO: 4 or 14, or which is degenerate therewith, or which has at least 70% sequence identity to SEQ ID NO: 4 or 14;
(ii) a nucleotide sequence which hybridizes to SEQ ID NO: 4 or 14 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 5 or 15 or an amino acid sequence which is at least 70% identical to SEQ ID NO: 5 or 15.

Thus, in the context of (a), (b) and (c) above, the method may involve the introduction of a single nucleic acid molecule encoding, e.g. crtE2, crtYh and crtYg (or proteins with the equivalent functional activity) from either the NCTC2665 or preferably the Otnes 7 strains of M. luteus. Alternatively, two or more separate molecules may be introduced.

A lycopene-producing host cell may be any cell that is capable of producing lycopene, preferably in significant amounts. A lycopene-producing cell comprises the biosynthetic machinery necessary to produce lycopene, either naturally or by introduction into the host cell. For example, the sarcinaxanthin biosynthetic machinery comprises genes encoding enzymes capable of producing lycopene, i.e. crtE, crtB and crtl. Thus, the method may include the introduction and expression of one or more nucleic acid molecules comprising a nucleotide sequences as set forth in all or part of any one of SEQ ID NOs: 18, 20, 22, 27, 29 and 31, or which are degenerate therewith, or which are at least 70% identical to SEQ ID NOs: 18, 20, 22, 27, 29 or 31, or which are otherwise related to SEQ ID NOs 18, 20, 22, 27, 29 or 31 by analogy to the definitions given above in relation to SEQ ID NOs. 2, 4, 12 or 14 or their corresponding amino acid sequences. Alternatively, the endogenous lycopene biosynthetic machinery of the host cell may be modified so as to enhance lycopene production in said host.

Preferably the lycopene producing host cell comprises genes encoding the crtE, crtB and crtl proteins from Pantoea ananatis or parts or functional equivalents thereof, wherein said genes are expressed. In other words, the host cell comprises genes encoding three enzymes for the biosynthesis of lycopene from isoprenyl pyrophosphate (IPP) and dimethylallyl pyrophosphate (DMAPP). Said genes may be integrated into the host genome or present in the form of a plasmid or equivalent thereof. Conveniently, the lycopene producing host cell may comprise the plasmid pAC-LYC (Cunningham and Gantt, 2007).

As discussed above, enzymes capable of catalysing the conversion of lycopene to flavuxanthin, i.e. lycopene elongases, are known in the art, e.g. crtEb from Corynebacterium glutamicum, and nucleic acid molecules encoding any enzymes with an equivalent functional activity may be used in the sarcinaxanthin production methods. Preferably the nucleic acid molecule encoding a protein capable of catalysing the conversion of lycopene to flavuxanthin may be a nucleic acid molecule comprising:
(i) a nucleotide sequence as set forth in all or part of SEQ ID NO: 6, 7 or 10, or which is degenerate therewith, or which has at least 70% sequence identity to SEQ ID NO: 6, 7 or 10;
(ii) a nucleotide sequence which hybridizes to SEQ ID NO: 6, 7 or 10 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 8, 9 or 11 or an amino acid sequence which is at least 70% identical to SEQ ID NO: 8, 9 or 11.

As described in the examples, the sarcinaxanthin biosynthetic gene cluster encodes a sarcinaxanthin glycosylase enzyme, which activity results in the production of both sarcinaxanthin mono- and diglucosides. Thus, the method may include the introduction of a further nucleic acid molecule into said host cell to produce such glucosides, wherein said nucleic acid molecule encodes an enzyme capable of glycosylating sarcinxanthin, such as crtX from M. luteus or a functional equivalent thereof. Most preferably, the nucleic acid comprises:
(i) a nucleotide sequence as set forth in all or part of SEQ ID NO: 16, or which is degenerate therewith, or a nucleotide sequence with at least 70% sequence identity to SEQ ID NO: 16;
(ii) a nucleotide sequence which hybridizes to SEQ ID NO: 16 under non-stringent binding conditions of 6×SSC/50% formamide at room temperature and washing under conditions of high stringency, e.g. 2×SSC, 65° C., where SSC=0.15 M NaCl, 0.015M sodium citrate, pH 7.2; or
(iii) a nucleotide sequence encoding a protein having all or part of an amino acid sequence as set forth in SEQ ID NO: 17 or which comprises an amino acid sequence which is at least 70% identical to SEQ ID NO: 17.

Alternatively, sarcinaxanthin produced according to the above methods may be glycosylated by glycosylase enzymes or other glycosylation mechanisms which are present in the host cell. Further, the sarcinaxanthin produced according to the invention may be glycosylated in vitro according to procedures well known in the art.

Generally speaking to perform the methods of production an appropriate expression vector may include appropriate control sequences such as for example translational (e.g. start and stop codons, ribosomal binding sites) and transcriptional control elements (e.g. promoter-operator regions, termination stop sequences) linked in matching reading frame with the nucleic acid molecules required for performance of the method as described herein. Appropriate vectors may include plasmids and viruses (including, e.g. bacteriophage). Preferred vectors include bacterial expression vectors, e.g. pBAD-vectors, pET-vectors and pTRC-vectors. The nucleic acid molecule may conveniently be fused with DNA encoding an additional polypeptide, e.g. glutathione-S-transferase, to produce a fusion protein on expression.

A range of vectors are possible and any convenient or desired vector may be used. Vectors may be used which are based on the broad-host-range RK2 replicon, into which an appropriate strong promoter may be introduced. For example WO 98/08958 describes RK2-based plasmid vectors into which the Pm/xylS promoter system from a TOL plasmid has been introduced. Other vectors or expression systems which may be used include for example those based on the pET, pBT, pMyr, pSos, pTRG or pGen expression systems. Promoters that may be useful in the expression of the proteins according to the invention include, but are not limited to, the lac promoter, T7, Ptac, PtrcT7 RNA polymerase promoter (P₇φ10), λP_{L} and P_{BAD}. Any suitable expression system may be used in the host cell and will be dependent on the nature of said cells. Preferably the nucleic acid molecules used in the methods discussed above are under the control of the Pm/xylS promoter system.

Generally speaking, the nucleic acid molecule will be expressed in a host cell under conditions in which the biosynthetic machinery may be expressed.

The methods further comprise the step of recovering (e.g. isolating or purifying) sarcinaxanthin, e.g. from the culture medium in which the host cell was grown or from the host cell. This can be isolated or purified from the cell culture medium into which it has been transported or secreted if appropriate, or otherwise from the host cell in which it has been produced. Thus, for example, the cells of the producing organism may be harvested, e.g. by centrifugation, and sarcinaxanthin or a derivative thereof may be extracted following cell lysis, for example with organic solvent(s) (e.g., methanol and acetone in a ratio of 7:3). The sarcinaxanthin or derivatives thereof may be recovered from such an extract, for example by precipitation or evaporation. Further purification of a crude product obtained in this way may include e.g. chromatography, e.g. HPLC.

By way of representative example, the crtE2YgYh regions of the M. luteus strain Otnes 7, may be amplified from genomic DNA and inserted into an expression vector, e.g. pJBphOx. Said expression vector may then be introduced into a host cell, e.g. E. coli XL1 Blue containing the pAC-LYC plasmid (described above). The host cell may then be cultivated such that the proteins encoded by the pAC-LYC and expression vectors are expressed thereby resulting in the production of sarcinaxanthin.

The host cell may be any desired cell or organism, prokaryotic or eukaryotic, but generally it will be a microorganism particularly a bacterium. More particularly, the host cell will be an Escherichia coli cell or a Corynebacterium glutamicum cell.

The novel isolated strain referred to above, from which the gene cluster was also sequenced (isolate Otnes 7), as deposited under deposit number DSM 23579 at the DSMZ, may be used for the production of sarcinaxanthin, but is not a preferred host cell for the methods. However, this strain is a preferred source of the nucleic acid molecules for use in the methods.

The sarcinaxanthin produced by these methods may be further modified for example by glycosylation or other derivatisation, in order to exhibit or improve activity, e.g. antioxidant activity. Methods for glycosylating carotenoids are generally known in the art; the glycosylation may be effected intracellularly by providing the appropriate glycosylation enzymes or may be effected in vitro using chemical synthetic means.

Mutations can be made to the native sequences using conventional techniques.

The method below illustrates how sarcinaxanthin may be generated and isolated using the above described general methodology.

### Materials and Methods

### Bacteria, Plasmids, Standard DNA Manipulations, and Growth Media

Bacterial strains and plasmids used in this work are listed in Table 2. Bacteria were cultivated in Luria-Bertani (LB) broth (Sambrook et al., 1989), and recombinant E. coli cultures were supplemented with ampicillin (100 μg/ml) and chloramphenicol (30 μg/ml). M. luteus and C. glutamicum strains were grown at 30° C. and 225 rpm agitation, while E. coli strains were generally grown at 37° C. and 225 rpm agitation. For heterologus production of carotenoids, 250 ml cultures of recombinant E. coli strains were grown at 28° C. with 180 rpm agitation in 500 ml Erlenmeyer shake flasks for 24 h in the presence of 0.5 mM of the Pm inducer m-toluate, unless otherwise stated. Standard DNA manipulations were performed according to Sambrook et al., (1989) and isolation of total DNA from M. luteus strains was performed as described elsewhere (Tripathi and Rawal, 1998).

### Vector Constructions

pCRT-EBIE2YgYh-2665 and pCRT-EBI-2665:

The complete crtEBIE2YgYh gene cluster of M. luteus NCTC2665 was PCR amplified from genomic DNA by using the primer pair crtE-F (5′-TTTTTCATATGGGTGAAGCGAGGACGGG-3′) and crtYh-R (5′-TTTTTGCGGCCGCTCAGCGATCGTCCGGGTGGGG-3′). The crtEBI region of M. luteus NCTC2665 was PCR amplified from genomic DNA by using the primer pair crtE-F (see above) and crtl-R (5′-TTTTTGCGGCCGCTCATGTGCCGCTCCCCCCGG). The resulting PCR products, crtEBIE2YgYh (5283 bp) and crtEBI (3693 bp), were end digested with NdeI and NotI (indicated in bold in primer sequences) and ligated into the corresponding sites of pJBphOx (Sletta et al., 2004), yielding plasmids pCRT-EBIE2YgYh-2665 and pCRT-EBI-2665, respectively.

pCRT-E2YgYh-2665 and pCRT-E2YgYh-O7:

The crtE2YhYg regions of M. luteus strains NCTC2665 and Otnes7 were PCR amplified from genomic DNA using primers crtE2-F (5′-TTTTTCATATGATCCGCACCCTCTTCTG-3′) and crtYh-R (see above). The obtained 1615 bp PCR products were blunt end ligated into pGEM-Teasy vector system (Promega, Madison, Wis.), and the resulting plasmids were digested with NdeI and NotI and the 1597 bp inserts were ligated into the corresponding sites of pJBphOx, yielding plasmids pCRT-E2YgYh-2665 and pCRT-E2YgYh-O7, respectively.

pCRT-E2YgYhX-O7:

The crtE2YgYhX region of M. luteus strain Otnes7 was PCR amplified from genomic DNA using primers crtE2-F (see above) and crtYX-R: (5′-TTTTTCCTAGGAGATGGCCGCGAACATCCTG). The obtained PCR product was end digested with NdeI and BlnI (indicated in bold in the primer) and the corresponding 3085 bp fragment ligated into the corresponding sites of pJBphOx, resulting in pCRT E2YgYhX-O7.

pCRT-E2Yq-O7 and pCRT-E2Yq-2665:

The crtE2Yg coding regions of M. luteus strains NCTC2665 and Otnes7 were PCR amplified from chromosomal DNA using primers crtE2-F (see above) and crtYg-R (5′-TTTTTGCGGCCGCTCACCGGCTCCCCCGGTCGGTC-3′). The obtained PCR products were end digested with NdeI and NotI (indicated in bold in primer sequence) and resulting 1247 bp fragments ligated into the corresponding sites of pJBphOx, resulting in pCRT-E2Yg-O7 and pCRT-E2Yg-2665, respectively.

pCRT-E2-O7 and pCRT-E2-2665:

The crtE2 genes of M. luteus strains NCTC2665 and Otnes7 were PCR amplified from chromosomal DNA using primers crtE2-F (see above) and crtE2-R (5′-TTTTTGCGGCCGCTCATGCCGCCGCCCCCCGGG-3′). The resulting PCR products were end digested with NdeI and NotI (indicated in bold in the primer sequence) and the corresponding 890 bp fragments ligated into likewise treated pJB658phOx, resulting in pCRT-E2-O7 and pCRT-E2-2665, respectively.

pCRT-YgYh-O7 and pCRT-YgYh-2665:

The YgYh regions of M. luteus strains NCTC2665 and Otnes7 were PCR amplified from genomic DNA by using primers crtYg-F (5′-TTTTTCATATGATCTACCTGCTGGCCCT-3′) and crtYh-R (see above). The resulting 734 bp PCR products were end digested with digested with NdeI and NotI (indicated in bold in the primer sequences) and resulting 716 bp fragments were ligated into the corresponding sites of pJB658phOx, resulting in pCRT-YgYh-O7 and pCRT-YgYh-2665, respectively.

pCRT-E2YeYf-Hybrid:

According to the gene sequences of crtE2 in M. luteus Otnes7 and crtYeYf in C. glutamicum MJ233-MV10, four primers crtE2-F (5′-TGACCAACGACCGGTAGCGGAG-3′) and crtE2-1-R (5′-CCCATCCACTAAACTTAAACATCATGCCGCCGCCCCCCGG-3′), crtYe-1-F (5′-TGTTTAAGTTTAGTGGATG GGTTGATCCCTATCATCGATATTTCAC-3′) and crtYf-R (5′-TTTTGCGGCCGCTTTTCCATCATGACTACGGCTTTTC) were used. Primers crtE2-i-R and crtYe-i-F contain homologous extensions of 21 bp (italic) at the 5′ ends as linker sequences in order to allow cross over PCR. Primer pair crtE2-F and crtE2-i-R was used to amplify a 1227 bp fragment containing the crtE2 gene from genomic M. luteus DNA and primer pair crtYe-i-F and crtYf-R was used to amplify a 885 bp crtYeYf containing fragment from genomic C. glutamicum DNA. The resulting PCR fragments were used as template for PCR with primer pair crtE2-F and crtYe-R to amplify a 2090 bp hybrid DNA fragment containing crtE2 from M. luteus and crtYeYf from C. glutamicum connected by the 21-bp linker sequence. The resulting hybrid fragment was end digested with AgeI and NotI (indicated in bold in primer sequence) and the obtained 2070 bp DNA fragment ligated into the corresponding sites of pJB658phOx, resulting in vector pCRT-E2YeYf-Hybrid.

pCRT-YeYfEb-MJ:

The crtYeYfEb genes from C. glutamicum strain MJ-233C-MV10 were PCR amplified from genomic DNA using primers crtYe-F1 (5′-TGGCTATCTCTAGAAAGGCCTACCCCTTAGGCTTTATGCAACAGAAACAATAAT AATGGAGTCATGAACATATGATCCCTATCATCGATATTTCAC-3′) and crtYf-R (5′-TTTTGCGGCCGCCTGATCGGATAAAAGCAGAGTTATATC-3′). The resulting PCR product was digested with XbaI and NotI (indicated in bold in primer sequence) and the resulting 1789 bp DNA fragment was ligated into the corresponding sites of pJBphOx, yielding pCRT-YeYfEb-MJ.

All the constructed vectors were verified by DNA sequencing and transformed by electroporation (Dower et al., 1988) into E. coli strain XL1-blue and the lycopene producing E. coli strain XL1-blue (pAC-LYC), respectively (Cunningham et al., 1994).

Extraction of Carotenoids from Bacterial Cell Cultures

To extract carotenoids from M. luteus strains, cells were harvested, washed with deionized H₂O, treated with lysozyme (20 mg/ml) and lipase (Fluka Chemicals, Germany) according to (Kaiser et al., 2007) and the pigments were extracted with a mixture of methanol and acetone (7:3). For recombinant E. coli strains, 50 ml aliquots of the cell cultures were centrifuged at 10,000×g for 3 min and the pellets were washed with deionized H₂O, the cells were then frozen and thawed to facilitate extraction. Finally the pigments were extracted with 4 ml methanol/acetone at 55° C. for 15 min with thorough vortex every 5 min. When necessary, up to three extraction cycles were performed to remove all colours from the cell pellet. When selective extraction for xanthophylls was desired, pure methanol was used. 0.05% butylhydroxytoluene (BHT) was added to the organic solvent to contribute to the stabilization of carotenoids. Samples for preparative HPLC were in addition partitioned into 50% diethyl ether in petroleum ether. The collected upper phase was evaporated to dryness and dissolved in methanol.

### Quantification of Carotenoids in Cell Extracts

Carotenoids were quantified on the basis of the area in the chromatographic analysis and by using a standard curve made by known concentrations of a trans-beta-apo-8′-carotenal and lycopene standard (Fluka). The correct concentrations of the standard was determined spectrophotometrically (Harker and Bramley, 1999) by using the extinction coefficients E 1 cm 1% of 3450 for lycopene and 2590 for apo-carotenal. Standards were filtered through a syringe 0.2 μm polypropylene filter (Pall Gelman) and stored in amber glass vessels at −80° C. under N₂ atmosphere if not analyzed immediately.

### LC-Ms Analyses

LC-MS analyses were performed on an Agilent Ion Trap SL mass spectrometer equipped with an Agilent 1100 series HPLC system. The HPLC system was equipped with a diode array detector (DAD) which recorded UV/VIS spectra in the range from 200-650 nm. Two HPLC protocols were used for the analysis in this work. A high throughput protocol for a fast quantitative determination of known carotenoids was used as follows; the carotenoids were eluted isocratically in MeOH for 5 min. A Zorbax rapid resolution SB RP C₁₈ column with dimension 2.1*30 mm was used for the analyses. Column flow was kept at 0.4 mL/min and 10 μL extract was injected for each run. For detailed qualitative carotenoid separation a Zorbax SB RP C₁₈ with dimension 2.1*150 mm was used. The carotenoids were eluted isocratically in MeOH/Acetonitrile (7:3) for 25 minutes. The column flow was 250 μl/min and 10 or 20 μL sample was injected depending on the concentration.

For determination of the molecular masses of carotenoids, mass spectrometry (MS) was performed under the following conditions. Analytes were ionized using a chemical ionization source with settings 325° C. dry temperature, 350° C. vaporizer temperature, 50 psi nebulizer pressure and 5.0 L/min dry gas. The MS was operated in scan mode. For carotenoid identification, preparative HPLC was performed on an Agilent preparative HPLC 1100 series system equipped with two preparative HPLC pumps, a preparative autosampler and a preparative fraction collector. Mobile phases were methanol in channel 1 and acetonitrile in channel 2. Samples of 2 mL were injected at a flow rate of 20 mL/min to a Zorbax RP C18 2.1*250 mm preparative LC column. On-line MS analysis was performed by splitting the flow 1:200 after the column using an Agilent LC flow splitter and a make-up flow of 1 mL methanol/min was used to carry the analytes to the MS with less than 15 sec delay. The diode array detector was used to trigger fraction collection.

### Carotenoid Structure Determination by NMR

All NMR spectra were recorded on a Bruker Avance 600 MHz instrument, fitted with a TCl cryoprobe using CDCl₃ as solvent with TMS as internal reference.

¹H and ¹³C signals were unambiguously assigned by the aid of ip-COSY, HSQC, HMBC, NOESY and HSQC-TOCSY experiments.

### Results

### Analysis of Carotenoids Produced by M. Luteus Strains NCTC2665 and Otnes7

We initially characterised the major carotenoids synthesized by M. luteus, and the recently genome sequenced M. luteus NCTC2665 was chosen as one model strain. Cell extracts from shake flask cultures were analyzed by LC-MS and one major peak (peak 3) (FIG. 4A) was identical to that of the sarcinaxanthin standard purified and structurally identified by NMR earlier M. luteus (Stafsnes et al., 2010). In addition, two minor peaks, peak 1 and peak 2, were identified with the same absorption spectra as that of sarcinaxanthin (FIG. 4A). The retention time of peak 2 was equal to sarcinaxanthin monoglucoside identified by NMR earlier (Stafsnes et al., 2010), while peak 1 was more polar and therefore here predicted to represent sarcinaxanthin diglucoside (Table 3).

Several M. luteus strains from the sea surface microlayer of the mid-part of the Norwegian coast has previously been isolated and characterized for their sarcinaxanthin production capacities (Stafsnes et al., 2010). One selected isolate, designated Otnes7, forms bright yellow colonies on LB agar plates and with higher colour intensity than that of strain NCTC2665. Otnes7 was here classified as a M. luteus strain by 16S-rRNA sequence analysis (93% identical to NCTC2665), and this strain was included as a second model strain. Qualitative analysis of extracts confirmed that strain Otnes7 produces the same carotenoids as NCTC2665, while the total carotenoid level (190 μg/g CDW) of Otnes7 cells was higher than that of NCTC2665 cells (145 μg/g CDW). The latter result was in agreement with the different colour intensities of the respective bacterial colonies, and this was further investigated.

Cloning and Genetic Characterisation of the M. Luteus NCTC2665 crtEIBE2YgYh Sarcinaxanthin Biosynthetic Gene Cluster

The genome sequence of M. luteus strain NCTC2665 was deposited in the databases (Accession number: NC_{—}012803). In silico screening of the DNA sequence data resulted in identification of a putative carotenoid biosynthesis gene cluster consisting of eight open reading frames, or1007, or1009-or1014 and ORF1. The genetic organization of crt genes in M. luteus displayed certain similarities to the previously published biosynthetic gene clusters for the C₅₀ carotenoids C.p.450 and decaprenoxanthin in Dietzia sp. (Tao et al., 2007) and C. glutamicum (Krubasik, Kobayashi et al. 2001), respectively (FIG. 5).

Expression of the crtEIBE2YgYh Genes Resulted in Production of Non-Glycosylated Sarcinaxanthin in E. coli

To experimentally test if the identified M. luteus gene cluster encoded an active sarcinaxanthin biosynthetic pathway, the crtEBIE2YgYh region from NCTC2665 was cloned in frame and under transcriptional control of the positively regulated Pm promotor in plasmid pJBphOx (Sletta et al., 2004). This expression vector has many favourable properties useful for regulated expression of genes and pathways under relevant levels in gram-negative bacteria (for review, see Brautaset et al., 2009). The resulting plasmid pCRT-EBIE2YgYh-2665 was transformed into the non-carotenogenic E. coli host strain XL1-blue, and the recombinant strain was analysed for carotenoid production under induced conditions (0.5 mM m-toluic acid). LC-MS analysis of cell extracts revealed a small peak at identical retention time, absorption spectrum, and relative molecular mass as sarcinaxanthin identified in M. luteus strains. The recombinant E. coli strain produced small amounts of sarcinaxanthin (10 to 15 μg/g CDW), which was not present in plasmid free cells, confirming that the identified gene cluster encodes a sarcinaxanthin biosynthetic pathway from FFP.

Sarcinaxanthin Production Levels can be Increased Up to 150-Fold by Expressing Otnes7 crtE2YgYh Genes and in a Lycopene Producing E. coli Host

To overcome the poor sarcinaxanthin production levels obtained (above) a recombinant strain E. coli XL1 Blue (pCRT-EBI-2665) was established, expressing three enzymes catalyzing the conversion of FFP into lycopene (FIG. 3). Analysis of this recombinant strain under induced conditions confirmed that it produced lycopene. However, the production levels (8-12 μg/g CDW) remained low; analogous with the sarcinaxanthin levels obtained with E. coli XL1 Blue (pCRT-EBIE2YgYh-2665) (see above). Therefore, E. coli XL1-blue was transformed with plasmid pAC-LYC (Cunningham and Gantt, 2007) harbouring the Pantoea ananatis crtEBI genes encoding three enzymes for biosynthesis of lycopene from IPP (isoprenyl pyrophosphate) and DMAPP (dimethylallyl pyrophosphate). LC-MS analysis confirmed that the resulting strain XL1-blue (pAC-LYC) accumulated significant amounts of lycopene (1.8 mg/g CDW) as sole carotenoid. Therefore, all further carotenoid production experiments were performed by using XL1-blue (pAC-LYC) as a host.

XL1-blue (pAC-LYC) (pCRT-E2YgYh-2665), and LC-MS analysis of cell extracts revealed a total carotenoid accumulation of 2.3 mg/g CDW and about 90% of the total carotenoid produced was identified as sarcinaxanthin. These data demonstrated that the M. luteus NCTC2665 crtE2YgYh gene products can effectively convert lycopene into sarcinaxanthin in a lycopene producing cell under these conditions. We also established and analysed the strain XL1-blue (pAC-LYC) (pCRT-EBIE2YgYh-2665) and the results were similar as for XL1-blue (pAC-LYC) (pCRT-E2YgYh-2665) strain. The latter result implies that the M. luteus crtEBI gene products are not efficient for lycopene production in E. coli, and whether this is due to poor expression levels or low catalytic activities in this host, remained unknown.

An analogous strain XL1 Blue (pAC-LYC) (pCRT-E2YgYh-O7) was established, and the total carotenoid production level (2.5 mg/g CDW) of the resulting recombinant strain was slightly higher than that of analogous strain XL1 Blue (pAC-LYC) (pCRT-E2YgYh-2665). 97% of the total carotenoid produced by XL1 Blue (pAC-LYC) (pCRT-E2YgYh-O7) was sarcinaxanthin indicating efficient conversion of the lycopene. It should also be noted that the sarcinaxanthin production levels obtained in this heterologous host was above 10-fold higher than those obtained by the two M. luteus strains under such conditions (see above). To further compare the efficiency of using Otnes7 versus NCTC2665 derived biosynthetic genes, production analyses were performed with different Pm inducer concentrations (FIG. 6). The results demonstrated that strain XL1-blue (pAC-LYC) (pCRT-E2YgYh-O7) produced sarcinaxanthin to significantly higher levels than strain XL1-blue (pAC-LYC) (pCRT-E2YgYh-2665) under all conditions tested, thus confirming that Otnes7 genes are preferable for efficient sarcinaxanthin production in an E. coli host. This result was in agreement with the higher sarcinaxanthin production levels of Otnes7 compared to NCTC2665 (see above). DNA sequence analysis of the cloned Otnes7 crtE2YgYh fragment revealed in total 24 nucleotide substitutions compared to the corresponding NCTC2665 DNA sequence, resulting in three amino acid substitutions in CrtE2, six in CrtYg, and two substitutions plus one insertion in CrtYh. It is proposed that one or more of these sequence variations positively affects the expression level or the catalytic properties of the respective proteins.

Expression of crtE2 and crtE2Y Resulted in Accumulation of C₄₅ Nonaflavuxanthin and C₅₀ Flavuxanthin

To elucidate the detailed biosynthetic steps for the conversion of lycopene to sarcinaxanthin, recombinant strain XL1 Blue (pAC-LYC) (pCRT-E2-2665) was established and analysed for carotenoid production. Two different carotenoids were accumulated in the cells in addition to lycopene (FIG. 4D); all three compounds shared identical UV/Vis profiles. No sarcinaxanthin was detected. The minor carotenoid had a molecular mass of 620 Da, indicating a C₄₅ carotenoid and the major carotenoid had a molecular mass of 704 Da indicating a C₅₀ carotenoid. The major carotenoid was separated by preparative HPLC and analyzed by NMR. Inspection of ¹H, ¹³C and HSQC spectra revealed chemical shifts in agreement with reported data for the acyclic C₅₀ carotenoid flavuxanthin (Krubasik, Takaichi et al. 2001). The minor carotenoid was identified as nonaflavuxanthin on the basis of the UV/Vis profile and the mass (Table 3). These results verified that the M. luteus crtE2 gene encodes a lycopene elongase catalyzing the sequential elongation of the C₄₀ carotenoid lycopene via the C₄₅ carotenoid nonaflavuxanthin to the C₅₀ carotenoid flavuxanthin. A similar analysis by using the analogous strain XL1 Blue (pAC-LYC) (pCRT-E2-O7) gave the same conclusion. Interestingly, the relative conversion of lycopene was substantially higher in the latter strain (79% vs. 23%), which was in agreement with the generally higher sarcinaxanthin production level obtained when expressing Otnes7 genes (see FIG. 6).

We then constructed and analysed recombinant strains XL1 Blue (pAC-LYC) (pCRT-E2Yg-O7) and XL1 Blue (pAC-LYC) (pCRT-E2Yg-2665). The carotenoids produced by both strains were flavuxanthin, nonaflavuxanthin and lycopene and their relative abundance was similar to strains XL1 Blue (pAC-LYC) (pCRT-E2-O7) and XL1 Blue (pAC-LYC) (pCRT-E2-2665), respectively. Taken together our data thus imply that the CrtYg and CrtYh polypeptides must function together as an active C₅₀ carotenoid cyclase catalyzing cyclization of flavuxanthin to sarcinaxanthin in vivo. To our knowledge, this γ-type of carotenoid cyclase enzyme has not previously been described. To unravel if this cyclase can also catalyse cyclization of lycopene, we established and analysed recombinant strains XL1 Blue (pAC-LYC) (pCRT-YgYh-O7) and XL1 Blue (pAC-LYC) (pCRT-YgYh-2665). HPLC analysis showed that both strains accumulated lycopene, confirming that the crtYgYh gene products can not use lycopene as a substrate in vivo.

The crtX Gene Product Encodes an Active Glycosyl Transferase that can be Used to Produce Monoglycosylated Sarcinaxanthin in E. Coli Host

Immediately downstream of crtYh there is a an ORF encoding a hypothetical protein, followed by or1007 which encodes a putative polypeptide sharing 43% primary sequence identity to the putative glycosyl transferase protein CrtX (FIG. 5) from Dietzia sp., suggested to be involved in the glycosylation of C.p.450 (Tao et al., 2007). To our knowledge, no analogous gene has been found in the C. glutamicum genome sequence and still this bacterium can synthesize glycosylated decaprenoxanthin (Krubasik, Takaichi et al., 2001). The or1007 gene was herein named crtX, and to unravel its biological function we constructed and analysed recombinant strain XL1 Blue (PAC-LYC) (pCRT-E2YgYhX-O7). The resulting HPLC profile (FIG. 4C) revealed sarcinaxanthin as the major carotenoid (peak 3), but an additional more polar carotenoid was eluted earlier (peak 2) which had an identical retention time and absorption spectrum to that of sarcinaxanthin monoglucoside from M. luteus Otnes 7 (FIGS. 4C and E). Another minor peak was observed with the same retention time as that of sarcinaxanthin diglucoside; however, the detected amount was too low for a confident analysis of the mass and absorption spectrum. Interestingly, about 10% of the total produced sarcinaxanthin was glycosylated both in M. luteus and when produced heterologously in E. coli. These results confirmed that crtX encodes an active glycosyl transferase that is necessary for the glycosylation of sarcinaxanthin under the conditions tested.

Based on all accumulated data we could deduce the complete biosynthetic pathway of sarcinaxanthin and its glucosides from FFP and via lycopene in M. luteus (FIG. 3), and this represents to our knowledge the first experimentally confirmed biosynthetic pathway of a γ-cyclic C₅₀ carotenoid.

TABLE 2
Bacterial strains and plasmids used for heterologous production of
sarcinaxanthin and other C₅₀ carotenoids

| | | Reference |
|---|---|---|
| Strain/Plasmid | Relevant characteristics | source |
| Strain | | |
| E. coli DH5α | General cloning host | Gibco-BRL |
| E. coli XL1-blue | General cloning host | Stratagene |
| M. luteus | | National collection |
| NCTC2665 | | of Type Cultures |
| M. luteus Otnes7 | Marine wild type isolate | This work |
| C. glutamicum | Tn31831 mutant of | (Kurusu et al., |
| MJ-233C- | C. glutamicum MJ-233C; | 1990; Vertes et al., |
| MV10 | contains wild type | 1994; Krubasik, |
| | crt gene cluster | Takaichi et al., |
| | | 2001) |
| Plasmid | | |
| pGEM-T | Amp^{r}; Standard cloning | Promega, |
| | vector | Madison, USA |
| pJBphOx | Amp^{r}, pJB658 derivative | (Sletta et al., 2004 |
| pAC-LYC | Cm^{r}, lycopene producing | (Cunningham et |
| | plasmid containing crtEIB | al., 1993) |
| | from P. ananatis, p15A ori | |
| pGEM- | Amp^{r}, pGEM-T with | This work |
| TcrtE2YgYh-O7 | crtE2YgYh fragment | |
| | from strain Otnes7 | |
| pGEM-Tcrt | Amp^{r}, pGEM-T with | This work |
| E2YgYh-2665 | crtE2YgYh fragment from | |
| | strain NCTC2665 | |
| pCRT- | Amp^{r}, pJBphOx with phOx | This work |
| EBIE2YgYh- | fragment substituted with | |
| 2665 | crtEBIE2YgYh fragment | |
| | from strain Otnes7 | |
| pCRT-EBI-2665 | Amp^{r}, pJBphOx with phOx | This work |
| | fragment substituted with | |
| | crtEBI fragment from | |
| | strain NCTC 2665 | |
| pCRT-E2YgYh- | Amp^{r}, pJBphOx with phOx | This work |
| O7 | fragment substituted with | |
| | crtE2YgYh fragment | |
| | from strain Otnes7 | |
| pCRT-E2YgYh- | Amp^{r}, pJBphOx with phOx | This work |
| 2665 | fragment substituted with | |
| | crtE2YgYh fragment | |
| | from strain NCTC 2665 | |
| pCRT-E2Yg-O7 | Amp^{r}, pJBphOx with phOx | This work |
| | fragment substituted with | |
| | crtE2Yg fragment | |
| | from strain Otnes7 | |
| pCRT-E2Yg- | Amp^{r}, pJBphOx with phOx | This work |
| 2665 | fragment substituted | |
| | with crtE2Yg fragment | |
| | from strain NCTC2665 | |
| pCRT-E2-O7 | Amp^{r}, pJBphOx with phOx | This work |
| | fragment substituted with | |
| | crtE2 fragment from | |
| | strain Otnes7 | |
| pCRT-E2-2665 | Amp^{r}, pJBphOx with phOx | This work |
| | fragment substituted with | |
| | crtE2 fragment from | |
| | strain NCTC2665 | |
| pCRT-YgYh-O7 | Amp^{r}, pJBphOx with phOx | This work |
| | fragment substituted with | |
| | crtYgYh fragment | |
| | from strain Otnes7 | |
| pCRT-YgYh- | Amp^{r}, pJBphOx with phOx | This work |
| 2665 | fragment substituted with | |
| | crtYgYh fragment | |
| | from strain NCTC2665 | |
| pCRT- | Amp^{r}, pJBphOx with phOx | This work |
| E2YgYhX- | fragment substituted | |
| O7 | with crtE2YgYhX fragment | |
| | from strain Otnes7 | |
| pCRT-E2-O7- | Amp^{r}, pJBphOx with phOx | This work |
| YeYf-MJ | fragment substituted with | |
| | crtE2 fragment from | |
| | strain Otnes7 and YeYf from | |
| | C. glutamicum MJ-233C- | |
| | MV10 | |
| pCRT-YeYfEb- | Amp^{r}, pJBphOx with phOx | This work |
| MJ | fragment substituted | |
| | with crtYeYfEb fragment | |
| | from C. glutamicum MJ- | |
| | 233C-MV10 | |
| pCRT-E2Yg- | Ampr, pJBphOx with phOx | This work |
| 2665-Yf-MJ | fragment substituted with | |
| | a crtE2Yg fragment | |
| | from strain Otnes7 and | |
| | crtYf fragment from | |
| | C. glutamicum | |

TABLE 3
Characteristics of carotenoids extracted from M. luteus strain Otnes7 and
carotenoids produced heterologously with E. coli strains^{a}.

| | λₘₐₓ (nm) in | | | Relative | Retention |
|---|---|---|---|---|---|
| Carotenoid | the HPLC | | | molecular | time |
| (trivial name) | eluent | | | mass (m/z) | Rₜ (min) |
| Sarcinaxanthin | 414 | 438 | 467 | 1028 | 3.0 |
| diglucoside | | | | | |
| Sarcinaxanthin | 414 | 438 | 467 | 886 | 4.5 |
| monoglucoside | | | | | |
| Sarcinaxanthin | 414 | 438 | 467 | 704 | 7.7 |
| Flavuxanthin | 445 | 470 | 501 | 704 | 8.2 |
| Nonaflavuxanthin | 445 | 470 | 501 | 620 | 13.2 |
| Lycopene | 445 | 470 | 501 | 536 | 21.3 |
| Decaprenoxanthin | 414 | 438 | 467 | 704 | 10.1 |

^{a}Carotenoids dissolved in MeOH and separated by HPLC using the system including the Zorbax C18 150*30 column

### Example 3

### Efficacy of Irradiation Absorption Using an In Vitro Skin Model

### Method

The in vitro method of Springsteen was used (Springsteen et al., 1999, Analytica Chimica Acta, 380, p 155-164). Vitro-skin was used as the skin simulator and Miglyol (Miglyol 812F Neutraloel CHG.040906) or ethyl lactate (Sigma Aldrich) was used as the solvent. The tests were performed with a Varian Cary 300 Conc UV-Visible Spectrophotometer (with an integrating sphere). Sarcinaxanthin (prepared as described in Example 2) and the other carotenoids (Sigma Aldrich) were tested at various concentrations and immediately on application to the skin model or 10-20 minutes post-application.

### Results

FIGS. 7A-C show the irradiation absorption achieved by β-carotene (A), sarcinaxanthin (B) and zeaxanthin (C) at the concentrations indicated. The presented graphs also show the effects of the diluent on sarcinaxanthin absorption (FIG. 7B) and the effect of prolonged contact with the skin at 10, 15 or 20 minutes as indicated on the Figures. The results were compared to a conventional SPF 60 sun lotion and the use of diluent alone.

FIGS. 7A (β-carotene) and C (zeaxanthin) show only modest absorption in the 375-490 nm range, whereas sarcinaxanthin (FIG. 7B) shows strong absorption in this range. Furthermore, ethyl lactate proved to be the most suitable diluent (see FIGS. 7B and C). Finally, it will be noted that prolonged contact with the skin model led to loss of absorption in the case of β-carotene (FIG. 7A) and zeaxanthin (FIG. 7C), but not in the case of sarcinaxanthin (FIG. 7B).

A side-by-side comparison was conducted to further investigate the stability of the carotenoids after 15 minutes on the skin model. The results are shown in FIGS. 8A and B. FIG. 8A shows the results of absorption by the applied compounds immediately after application of the carotenoid to the skin model using ethyl lactate as diluent. After 15 minutes there is a significant difference in the absorption properties of β-carotene and zeaxanthin, both of which are unstable and lose most of their absorption properties in the relevant range. In contrast, sarcinaxanthin absorption appears unaffected by the prolonged contact demonstrating its superior stability compared to the other carotenoids.

### REFERENCES

Brautaset, T., Lale, R., and Valla, S. (2009). “Positively regulated bacterial expression systems.” Microbial Biotechnology 2: 15-30
Cunningham, F. X., Jr., D. Chamovitz, et al. (1993). “Cloning and functional expression in Escherichia coli of a cyanobacterial gene for lycopene cyclase, the enzyme that catalyzes the biosynthesis of beta-carotene.” FEBS Lett 328(1-2): 130-8
Cunningham, F. X., Jr. and E. Gantt (2007). “A portfolio of plasmids for identification and analysis of carotenoid pathway enzymes: Adonis aestivalis as a case study.” Photosynth Res 92(2): 245-59
Cunningham, F. X., Jr., Z. Sun, et al. (1994). “Molecular structure and enzymatic function of lycopene cyclase from the cyanobacterium Synechococcus sp strain PCC7942.” Plant Cell 6(8): 1107-21
Dower, W. J., J. F. Miller, et al. (1988). “High efficiency transformation of E. coli by high voltage electroporation.” Nucleic Acids Res 16(13): 6127-45
Harker, M. and P. M. Bramley (1999). “Expression of prokaryotic 1-deoxy-D-xylulose-5-phosphatases in Escherichia coli increases carotenoid and ubiquinone biosynthesis.” FEBS Lett 448(1): 115-9
Kaiser, P., P. Surmann, et al. (2007). “A small-scale method for quantitation of carotenoids in bacteria and yeasts.” J Microbiol Methods 70(1): 142-9
Krubasik, P., M. Kobayashi, et al. (2001). “Expression and functional analysis of agene cluster involved in the synthesis of decaprenoxanthin reveals the mechanisms for C50 carotenoid formation.” Eur J Biochem 268(13): 3702-8.
Krubasik, P., S. Takaichi, et al. (2001). “Detailed biosynthetic pathway to decaprenoxanthin diglucoside in Corynebacterium glutamicum and identification of novel intermediates.” Arch Microbiol 176(3): 217-23
Kurusu, Y., M. Kainuma, et al. (1990). “Electroporation-transformation system for coryneform bacteria by auxotrophic complementation.” Agric Biol Chem 54(2): 443-7
Sambrook, J., E. F. Fritsch, et al. (1989). “Molecular cloning: a Laboratory Manual”, 2nd edn. Cols Spring Harbor Laboratory Press, Cold Spring Harbor, N.Y.
Sletta et al., 2004 Appl. Env. Microbiol. 70(12):7033-7039
Stafsnes M H, J. K., Kildahl-Andersen G, Valla S, Ellingsen T E, Bruheim P. (2010). “Isolation and characterization of marine pigmented bacteria from Norwegian coastal waters and screening for carotenoids with UVA-blue light absorbing properties” The Journal of Microbiology 48(1): 16-23
Tao, L., H. Yao, et al. (2007). “Genes from a Dietzia sp. for synthesis of C40 and C50 beta-cyclic carotenoids.” Gene 386(1-2): 90-7
Tripathi, G. and S. K. Rawal (1998). “Simple and efficient protocol for isolation of high molecular weight DNA from Streptomyces aureofaciens.” Biotechnology Techniques 12(8): 629-631
Vertes, A. A., Y. Asai, et al. (1994). “Transposon mutagenesis of coryneform bacteria.” Mol Gen Genet. 245(4): 397-405

1. A method of treating or preventing the effects of irradiation in a human or non-human animal wherein a photoprotective composition comprising a carotenoid which has the formula: 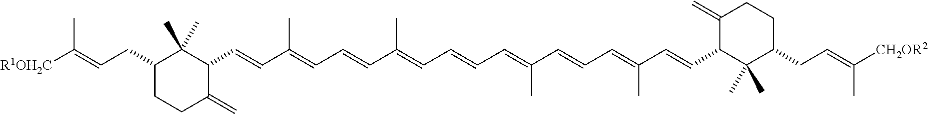
wherein R¹ and R², which may be the same or different, are each a hydrogen atom or a saccharide,
or a pharmaceutically acceptable derivative or salt thereof,
together with one or more pharmaceutically acceptable excipients and/or diluents,
is administered to said human or non-human animal.

2. A method as claimed in claim 1 wherein said saccharide is a monosaccharide.

3. A method as claimed in claim 2 wherein said monosaccharide is glucose or mannose.

4. A method as claimed in claim 1 wherein said carotenoid is sarcinaxanthin, 7,8-dihydrosarcinaxanthin, sarcinaxanthin succinate, sarcinaxanthin monoglucoside, sarcinaxanthin diglucoside or a pharmaceutically acceptable derivative or salt thereof.

5. A method as claimed in claim 1 wherein said pharmaceutically acceptable derivatives are cis- and trans-isomers, naturally occurring seco-, apo- and nor-carotenoid derivatives, epoxide derivatives, degradation products and dehydration derivatives, or pro-drugs.

6. A method as claimed in claim 1 wherein said carotenoid compound used in said composition is purified to a degree of purity of more than 30%.

7. A method as claimed in claim 1 wherein said carotenoid compounds are obtained or derived from naturally occurring sources.

8. A method as claimed in claim 1 wherein said carotenoid compounds are generated synthetically.

9. A method as claimed in claim 1 wherein said carotenoid compound is combined in the composition with additional sunscreen compounds.

10. A method as claimed in claim 9 wherein said composition contains two or more carotenoid compounds.

11. A method as claimed in claim 1 wherein said composition is in the form of a solution, suspension, gel, emulsion, ointment or cream.

12. A method as claimed in claim 1 wherein said composition optionally comprises additional sunscreen compounds wherein said composition is in the form of a gel, emulsion, ointment or cream.

13. A method as claimed in claim 1 wherein said composition is suitable for topical administration.

14. A method as claimed in claim 1 wherein said composition is formulated in a make-up product, a body product or a hair product and optionally comprises additional sunscreen compounds.

15. A method as claimed in claim 1 wherein said composition is administered in combination with one or more active ingredients which are effective in treating or preventing the effects of radiation.

16. A method as claimed in claim 1 wherein said composition is topically administered to the skin or hair of a human.

17. A method as claimed in claim 1 wherein said composition is photoprotective against light irradiation with a wavelength of 400-500 nm.

18. A photoprotective composition comprising a carotenoid which has the formula: 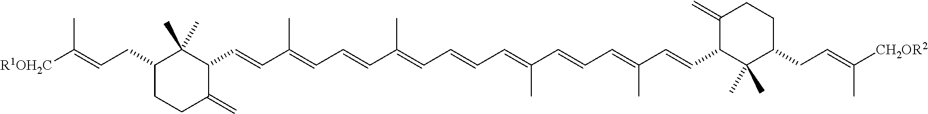
wherein R¹ and R², which may be the same or different, and are each a hydrogen atom or a saccharide,
or a pharmaceutically acceptable derivative or salt thereof,
together with one or more pharmaceutically acceptable excipients and/or diluents.

19. The photoprotective composition as claimed in claim 18 formulated as a cosmetic.

20. A photoprotective composition as claimed in claim 18 formulated as a medicament.

21. A method of treating or preventing the effects of irradiation in a human or non-human animal comprising administering to the human or non-human animal the photoprotective composition as claimed in claim 18.

22. A method of preparing a photoprotective or photoprotected product comprising applying a photoprotective compound as defined below 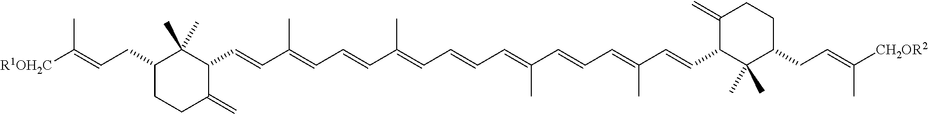
wherein R¹ and R², which may be the same or different, and are each a hydrogen atom or a saccharide,
or a pharmaceutically acceptable derivative or salt thereof,
to said product, or impregnating said product with said compound or a composition thereof.

23. A photoprotective or photoprotected product comprising a carotenoid which has the formula: 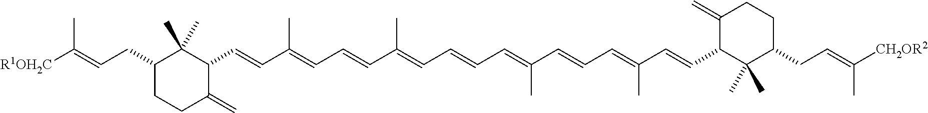
wherein R¹ and R², which may be the same or different, and are each a hydrogen atom or a saccharide,
or a pharmaceutically acceptable derivative or salt thereof.
